SEQUENCE LISTING (56 sequences)

SEQ ID NO: 1 - protein, 409 aa, Mycoplasma hominis
MSVFDSKFNGIHVYSEIGELETVLVHEPGREIDYITPARLDELLFSAILESHDARKEHQSFVKIMKDRGINVVELTDLVAETYDLASKAAKEEFIETFLEETVPVLTEANKKAVRAFLLSKPTHEMVEFMMSGITKYELGVESENELIVDPMPNLYFTRDPFASVGNGVTIHFMRYIVRRRETLFARFVFRNHPKLVKTPWYYDPAMKMPIEGGDVFIYNNETLVVGVSERTDLDTITLLAKNIKANKEVEFKRIVAINVPKWTNLMHLDTWLTMLDKNKFLYSPIANDVFKFWDYDLVNGGAEPQPQLNGLPLDKLLASIINKEPVLIPIGGAGATEMEIARETNFDGTNYLAIKPGLVIGYDRNEKTNAALKAAGITVLPFHGNQLSLGMGNARCMSMPLSRKDVKW

SEQ ID NO: 2 - protein, 409 aa, synthetic construct
  REGION 1..409: note = Mutated arginine deaminase
MSVFDSKFNGIHVYSEIGELETVLVHEPGREIDYITPARLDELLFSAILESHDARKEHQSFVKIMKDRGINVVELTDLVAETYDLASKAAKEEFIETFLEETVPVLTEANKEAVRAFLLSKPTHEMVEFMMSGITKYELGVESENELIVDPMPNLYFTRDPFASVGNGVTIHFMRYIVRRRETLFARFVFRNHPKLVKTPWYYDPAMKMSIEGGDVFIYNNETLVVGVSERTDLDTITLLAKNIKANKEVEFKRIVAINVPKWTNLMHLDTWLTMLDKNKFLYSPIANDVFKFWDYDLVNGGAEPQPQLNGLPLDKLLASIINKEPVLIPIGGAGATEMEIARETNFDGTNYLAIKPGLVIGYDRNEKTNAALKAAGITVLPFHGNQLSLGMGNARCMSMPLSRKDVKW

SEQ ID NO: 3 - protein, 392 aa, Mycoplasma phocicerebrale
IHVYSEIGELETVLVHEPGREIDYITPARLDELLFSAILESHDARKEHQSFVKQLKDNGINVVELTDLVAETFDLASKEEQEKLIEEFLEDSEPVLSEAHKTAVRKFLTSRKSTREMVEFMMAGITKYDLGIEADHELIVDPMPNLYFTRDPFASVGNGVTIHYMRYKVRQRETLFSRFVFSNHPKLVKTPWYYDPAMKMSIEGGDVFIYNNDTLVVGVSERTDLETITLLAKNIKANKEVEFKRIVAINVPKWTNLMHLDTWLTMLDKDKFLYSPIANDVFKFWDYDLVNGGAEPQPKENGLPLEGLLQSIINKKPVLIPIAGNNASHIDIERETHFDGTNYLAIKPGVVIGYARNEKTNAALAAAGIKVLPFHGNQLSLGMGNARCMSMP

SEQ ID NO: 4 - protein, 410 aa, Mycoplasma arginini
MSVFDSKFKGIHVYSEIGELESVLVHEPGREIDYITPARLDELLFSAILESHDARKEHKQFVAELKANDINVVELIDLVAETYDLASQEAKDKLIEEFLEDSEPVLSEEHKVVVRNFLKAKKTSRELVEIMMAGITKYDLGIEADHELIVDPMPNLYFTRDPFASVGNGVTIHYMRYKVRQRETLFSRFVFSNHPKLINTPWYYDPSLKLSIEGGDVFIYNNDTLVVGVSERTDLQTVTLLAKNIVANKECEFKRIVAINVPKWTNLMHLDTWLTMLDKDKFLYSPIANDVFKFWDYDLVNGGAEPQPVENGLPLEGLLQSIINKKPVLIPIAGEGASQMEIERETHFDGTNYLAIRPGVVIGYSRNEKTNAALEAAGIKVLPFHGNQLSLGMGNARCMSMPLSRKDVKW

SEQ ID NO: 5 - protein, 409 aa, Mycoplasma arthritidis
MSVFDSKFKGIHVYSEIGELETVLVHEPGKEIDYITPARLDELLFSAILESHDARKEHKEFVAELKKRGINVVELVDLIVETYDLASKEAKEKLLEEFLDDSVPVLSDEHRAAVKKFLQSQKSTRSLVEYMIAGITKHDLKIESDLELIVDPMPNLYFTRDPFASVGNGVTIHYMRYKVRQRETLFSRFVFSNHPKLVNTPWYYDPAEGLSIEGGDVFIYNNDTLVVGVSERTDLQTITLLAKNIKANKECEFKRIVAINVPKWTNLMHLDTWLTMLDKDKFLYSPIANDVFKFWDYDLVNGGDAPQPVDNGLPLEDLLKSIIGKKPTLIPIAGAGASQIDIERETHFDGTNYLAVAPGIVIGYARNEKTNAALEAAGITVLPFRGNQLSLGMGNARCMSMPLSRKDVK

SEQ ID NO: 6 - protein, 408 aa, Mycoplasma orale
  VARIANT 201: note = X can be any naturally occurring amino acid
  VARIANT 263: note = X can be any naturally occurring amino acid
  VARIANT 272: note = X can be any naturally occurring amino acid
  VARIANT 294: note = X can be any naturally occurring amino acid
SVFSDKFNGIHVYSEIGDLESVLVHEPGKEIDYITPARLDELLFSAILESTDARKEHKEFVEILKKQGINVVELVDLVVETYNLVDKKTQEKLLKDFLDDSEPVLSPEHRKAVEKFLKSLKSTKELIQYMMAGITKYDLGIKADKELIVDPMPNLYFTRDPFASVGNGVTIHYMRYKVRQRETLFSKFIFTNHPKLVKTPXYYDPAMKLSIEGGDVFIYNNDTLVVGVSERTDLETITLLAKNIKANKECEFKRIVAINVPKXTNLMHLDTXLTMLDKDKFLYSPIANDVFKFXDYDLVNGGSNPEPVVNGLPLDKLLESIINKKPVLIPIAGKGATEIETAVETHFDGTNYLAIKPGVVVGYSRNVKTNAALEANGIKVLPFKGNQLSLGMGNARCMSMPLSRKDVK

SEQ ID NO: 7 - protein, 391 aa, Mycoplasma gateae
IHVYSEIGELESVLVHEPGREIDYITPARLDELLFSAILESHDARKEHKLFVSELKANDINVVELTDLVTETYDLASQEAKDNLIEEFLEDSEPVLTEELKSVVRTYLKSIKSTRELIQMMMAGITKYDLGIEADHELIVDPMPNLYFTRDPFASVGNGVTIHYMRYKVRQRETLFSRFVFSNHPKLVNTPWYYDPSLKLSIEGGDVFIYNNNTLVVGVSERTDLETVTLLAKNIVANKECEFKRIVAINVPKWTNLMHLDTWLTMLDKDKFLYSPIANDVFKFWDYDLVNGGEEPQPVENGLPLEGLLESIINKKPILIPIAGEGASQIDIERETHFDGTNYLAIRPGVVIGYSRNEKTNAALEAAGIKVLPFHGNQLSLGMGNARCMSM

SEQ ID NO: 8 - protein, 392 aa, unidentified
  source 1..392: note = Mycoplasma phocidae
IHVYSEIGELQTVLVHEPGREIEYITPARLDELLFSAILESHDARKEHQEFVAELKKNNINVVELTDLVSETYDMVSKEKQEKLIEEFLEDSEPVLSEEHKGLVRKFLKSLKSSKELIQYMMAGITKHDLNIEADHELIVDPMPNLYFTRDPFASVGNGVTIHYMRYKVRQRETLFSRFIFANHPKLMNTPLYYNPDMKLSIEGGDVFVYNNETLVVGVSERTDLDTITLLAKNIKANKEREFKRIVAINVPKWTNLMHLDTWLTMLDKDKFLYSPIANDVFKFWDYDLVNGGDEPQPKVNGLPLEKLLESIINKKPILIPIAGTSASNIDVERETHFDGTNYLAIAPGVVIGYSRNVKTNEALEAAGIKVLPFKGNQLSLGMGNARCMSMP

SEQ ID NO: 9 - protein, 401 aa, Mycoplasma columbinum
MSKINVYSEIGELKEVLVHTPGDEIRRISPSRLDELLFSAILEPNEAIKEHKGFLKILQDKGIKVIQLSDLVAETYTYHATQKEREAFIEKWLDEAEPALTKDLRAKVKSYVLSKEGTPVAMVRTMMAGVSKQELNVESETELVVDPMPNLYFTRDPFASAGNGISLNNMKYVTRKRETIFAEFIFATHPDYKTTPHWFDRLDEGNIEGGDVFIYNKDTLVIGVSERTNKEAILTIAKKIKNNKEAKFKKIVAINVPPMPNLMHLDTWLTMVDKDKFLYSPNMLSVLKVWEIDLSKEIEMVETNKPLADVLESIIGVKPVLIPIAGKGATQLDIDIETHFDGTNYLTIAPGVVVGYSRNIKTEAALRAAGVTVLSFEGNQLSLGMGSARCMSMPLVREDVK

SEQ ID NO: 10 - protein, 407 aa, Mycoplasma iowae
MGNNIPKKINVFSEIGNLKRVLVHTPGKEIEYVTPQRLDELLFSAILDPVRAREEHKEFIKILESQGVEVVQLVDLTAETYDVAESQAKENFIQKWLDESLPKLTDENRNKVYSLLKSLEKDPKEMIRKMMSGVLASEIGVKSDVELIVDPMPNLYFTRDPFASVGNGITLHRMFRPTRRRETIFADFIFSNHPEYKSTQKYYEREDKFSLEGGDVFIYNNKTLVVGVSERTEKGAIKALAKAVQNNSNMSFEKIYAINVPKMSNLMHLDTWLTMLDTDKFLYSPNMMGVLKIWEIDLSDKSLKWKEIRDSLDHFLSTIIGKKAITVPVAGKDAMQFEIDIETHFDATNFIAVAPGVVIGYDRNKKTNEALKEAGIKVLSWNGDQLSLGMGSARCMTMPLYREELKK

SEQ ID NO: 11 - protein, 402 aa, Mycoplasma crocodyli
MNKINVYSEVGKLKEVLVHTPGDEIRRISPSRLEELLFSAILEPDSAIEEHKRFLKILEDNNIKVIQLDQLVADTYELVNPSVRDAFIEKWLNESEPKLDKKLREKVKEYLLHTQKTVGTKRMVRIMMAGVDRVELGVELDRQLVVDPMPNLYFTRDPFASAGNGISLNNMKYVTRKRETIFSEFIFENHPDYKTTPHWFDRLDKGNIEGGDVFIYNRTTLVIGISERTNKDALLTIANNIKSNKESKFERIVAVNVPPMPNLMHLDTWLTMVDHDKFLYSPNMMKTLKFWTIDLTKPIKMVELEESLSDMIETIIGKKPVLIPIAGHDASPLDVDIETHFDGTNYLTIAPGVVVGYSRNKLTEKALTKAGVKVLSFEGNQLSLGMGSARCMSMPLVREDIK

SEQ ID NO: 12 - protein, 430 aa, Mycoplasma fermentans
MQIIAKIDLLTNMLIFMKIYFIGRLIMKKINVYSEYGKLKEVLVHTPGDEIRRIAPSRLDELLFSAILEPDSAIAEHKRFVQLLKDNGIKVIQLDELFAKTFDLVSESVKQSLIERWLDECEPKLDATLRAKVKEYILELKAKSSKKMVRVMMAGIDKKELGIELDRDLVVDPMPNLYFTRDPFASVGNGISLHHMKYVTRQRETIFSEFIFDNNLDYNTVPRWFDRKDEGRIEGGDVFIYSADTLVVGVSERTNKEAINVMARKIAADKEVKFKRIYAINVPPMPNLMHLDTWLTMLDKNKFLYSPNMLSVLKVWRIDLNDPDFVWHEIEGSLEEILEQIIGMKPILIPIAGKGASQLDIDIETHFDGTNYLTIAPSVVVGYSRNEKTEKALKAAKVKVLSFEGNQLSLGMGSARCMSMPLIREDIKKK

SEQ ID NO: 13 - protein, 452 aa, Mycoplasma penetrans
MVITIALNILNKIYFKPQNRSILKLYRLPSLCTQISIFIGGKMSSIDKNSLGNGINVYSEIGELKEVLVHTPGDEIRYTAPSRLEELLFSAVLKADTAIEEHKGFVKILQNNGIKVIQLCDLVAETYELCSKEVRNSFIEQYLDEALPVLKKEIRPVVKDYLLSFPTVQMVRKMMSGILANELNIKQDNPLIIDGMPNLYFTRDPFASMGNGVSINCMKYPTRKREVIFSRFVFTNNPKYKNTPRYFDIVGNNGTIEGGDIFIYNSKTLVIGNSERTNFAAIESVAKNIQANKDCTFERIVVINVPPMPNLMHLDTWLTMLDYDKFLYSPNMMNVLKIWEIDLNVKPVKFVEKKGTLEEVLYSIIDKKPILIPIAGKGANQLDIDIETHFDGTNYLTIAPGVVVGYERNEKTQKALVEAGIKVLSFNGSQLSLGMGSARCMSMPLIRENLKK

SEQ ID NO: 14 - protein, 403 aa, Mycoplasma gallisepticum
MFNKIRVYSEIGKLRKVLVHTPGKELDYVTPQRLDELLFSSLLNPIKARQEHETFIKLLEDHDVECVQLSTLTAQTFQAVNSKIQEEFINRWLDECLPVLSEINRLKVYDYLKSLATNPQVMIRKMMSGILAKEVGIQSEVELVADPMPNLYFTRDPFASIGKGITLHSMFHPTRKRETIFADFIFSHHPEYKNAPKYYSREDKYSIEGGDLFVYDDKTLVIGVSERTEKKAIQSLAEKLRQNDETSFEKIYAINVPKMSNLMHLDTWLTMLDYDKFLYSPNMMGVLKIWEIDLIHPTLIWRELNESLEGFLSMVIGKKATLIPVAGEDSTQIEIDVETNFDATNFLVIQPGVVVGYDRNYKTNQALRDAGVKVISWNGDQLSLGMGSARCMSMPLYRDPIKK

SEQ ID NO: 15 - protein, 402 aa, Mycoplasma alligatoris
MSKINVYSEVGRLKEVLVHTPGDEIRRISPTRLEELLFSAILEPDTAIEEHKRFLNVLEKNGIKAIQLDELVAQTYDQVDQKIKDEFIDQWLQEAKPVLNDQLKKLVKNYLLKSQKEFSTKKMVRIMMAGIDKKEINIDLDRDLVVDPMPNLYFTRDPFASVGNGISLHNMKYQTRKRETIFAQFIFKYNKDYKTTPHWFDRFDHGSIEGGDVFVYTKDTLVIGISERTTKEAVLNIAKKIKANTDSKFKKIVAINVPPMPNLMHLDTWITMVDHDKFLYSPNMMKSLKFWLIDLSKEIKMVELEESLSNMLEAIIGKKPILIPIAGKNASQLDIDIETHFDGTNYLTIAPGVVVGYSRNKLTQKALEDAGVKVLSFDGNQLSLGMGSARCMSMPLVREDIK

SEQ ID NO: 16 - protein, 438 aa, Mycoplasma pneumoniae
MSKKQLVKTDGHNQLDQPNTKALQLKKKQFNSGVRVTSEISFLREVIAHHPGIETERVIDNQTFGSAMYLERAQKEHQLFIKILRQHGTKVHYLQDLLLEALSAADPNVRQDFIKNFLLESGIKSVSTFEACLNFFRSLDSLVDVIKVMFGGIKVSDVPPITPQRFADIHVSNSPFLIKPLSFSLYPHKFFNTLGTGVALFVTNDSELKRHSLVYEYIMRFHPRFDGVKLYTNRDFKNCLINSSDIIQISNEILLIGISHDTDVLGIESLARNLLSDHTNPIKQIIAINIHKFGAKTNLNKLIAMVDVDKFIIARKVLQATEIFELTATAQRDVDGLAQIKFKPLKFNFGEIIEAIIDKQPRFVIIGGGDEVAERKELLDCGMGVLNLSPGEIVVFDRNHYTNNLLNELGLIIHKIPASELSRGPSGPLEMVCSLWRE

SEQ ID NO: 17 - protein, 409 aa, Mycoplasma mobile
MKDTKDIINVFSEIGELKKVLIHTPGNELKYVSPYRLDELLFSNVLEWREAKKEHNEFIQKLKSEGVEPVELTDLVAESFEESSIKVKNDFIRQYLDEATPILDGLTKQKLLPFFLDIKHSTRKTIELMMSGITQKDISISHIERELIIDPMPNLYFSRDNFISIGNSVIISNMKYKTRKRETIFTDFIFKNHPLYKKVNMAFERKDLNNQISIIEGGDVLVYSKEILIIGISERTTMSAILELAENFKKTKRSFKKIYGVEVPKMKNLMHLDTWLTMIDYDKFIYSPNVLTDLKFWEINLDYEKISSKELHASLSEFLKLIIGKDPILIPIGGKGASQITIDIETNFVAANYLVIRPGVVIGYSRNYETQKALEGHGVKVIAFEGNQLSLGMGSSRCMSMPLIRSNLK

SEQ ID NO: 18 - protein, 411 aa, Streptococcus pyogenes
MTAQTPIHVYSEIGKLKKVLLHRPGKEIENLMPDYLERLLFDDIPFLEDAQKEHDAFAQALRDEGIEVLYLETLAAESLVTPEIREAFIDEYLSEANIRGRATKKAIRELLMAIEDNQELIEKTMAGVQKSELPEIPASEKGLTDLVESNYPFAIDPMPNLYFTRDPFATIGTGVSLNHMFSETRNRETLYGKYIFTHHPIYGGGKVPMVYDRNETTRIEGGDELVLSKDVLAVGISQRTDAASIEKLLVNIFKQNLGFKKVLAFEFANNRKFMHLDTVFTMVDYDKFTIHPEIEGDLRVYSVTYDNEELHIVEEKGDLAELLAANLGVEKVDLIRCGGDNLVAAGREQWNDGSNTLTIAPGVVVVYNRNTITNAILESKGLKLIKIHGSELVRGRGGPRCMSMPFEREDI

SEQ ID NO: 19 - protein, 408 aa, Enterococcus faecalis
MSHPINVFSEIGKLKTVMLHRPGKELENLMPDYLERLLFDDIPFLEKAQAEHDAFAELLRSKDIEVVYLEDLAAEALINEEVRRQFIDQFLEEANIRSESAKEKVRELMLEIDDNEELIQKAIAGIQKQELPKYEQEFLTDMVEADYPFIIDPMPNLYFTRDNFATMGHGISLNHMYSVTRQRETIFGQYIFDYHPRFAGKEVPRVYDRSESTRIEGGDELILSKEVVAIGISQRTDAASIEKIARNIFEQKLGFKNILAFDIGEHRKFMHLDTVFTMIDYDKFTIHPEIEGGLVVYSITEKADGDIQITKEKDTLDNILCKYLHLDNVQLIRCGAGNLTAAAREQWNDGSNTLAIAPGEVVVYDRNTITNKALEEAGVKLNYIPGSELVRGRGGPRCMSMPLYREDL

SEQ ID NO: 20 - protein, 403 aa, Mycoplasma capricolum
MEKKINVFSEIGTLKTVLVHRPGDEIENLTPELLERLLFDDVPFKDVAVKEHDAFTKIMRDNGVEVLYIEKLAAETLDQHPDLREKFIDQFISEANIEDKYKEKYRDFISSLDNYRMIKKMIAGTKKLELGIDEGYKAYPFIADPLPNVLFQRDPFSSVGFGITMNRMWSVTRNRETIFPDLVFKHHNRFANQVPYYYERDWKEETIEGGDILVLNKETLIIGVTQRTTLKAIEKFSERLFNDPESSYSKVIALDLPKSRAFMHLDTVFTNIDYDKFIAHPLIFDCIDEFKIYEVSKQGTKEVKKTLIELLSDAAGREVQIIRCGGNDVVGASREQWNDGTNVVALRPGKVIAYERNWITIDLLRKAGVEVLTIASSELSRGRGGPRCMTMPLWREDLQEIKR

SEQ ID NO: 21 - protein, 410 aa, Halothermothrix orenii
MFKKSPLNVTSEIGKLKKVLLHRPGHEIENLTPDLLERLLFDDIPYLKVAQEEHDAFAQTLRDNGVEVLYLHELAAEAIQEDEIRKKFIEQFLDEAGVIGKGARQVLKEYFADMDNETLIRKMMAGVRKKEIPAIEKVASLNDMVEEDYPFVLDPMPNLYFTRDPFATIGTGITLNHMRTETRNREVIFAEYIFSYHPDFKDTEIPFWFDRNETTSIEGGDELILSDKVLAMGISERTDAASIEKVARNIFTDGQPFETILAFKIPEKRAFMHLDTVFTMVDYDKFTIHAEIEGPLKVYSITKGDNDELKIDEEKATLEDTLKKYLGLDEVTLIRCAGGDYIDAGREQWNDGSNTLAIAPGEVVVYNRNHTTNRLLEEHGIKLHVIPSSELSRGRGGPRCMSMPLVREDI

SEQ ID NO: 22 - protein, 411 aa, Staphylococcus aureus
MTDGPIKVNSEIGALKTVLLKRPGKELENLVPDYLDGLLFDDIPYLEVAQKEHDHFAQVLREEGVEVLYLEKLAAESIENPQVRSEFIDDVLAESKKTILGHEEEIKALFATLSNQELVDKIMSGVRKEEINPKCTHLVEYMDDKYPFYLDPMPNLYFTRDPQASIGHGITINRMFWRARRRESIFIQYIVKHHPRFKDANIPIWLDRDCPFNIEGGDELVLSKDVLAIGVSERTSAQAIEKLARRIFENPQATFKKVVAIEIPTSRTFMHLDTVFTMIDYDKFTMHSAILKAEGNMNIFIIEYDDVNKDIAIKQSSHLKDTLEDVLGIDDIQFIPTGNGDVIDGAREQWNDGSNTLCIRPGVVVTYDRNYVSNDLLRQKGIKVIEISGSELVRGRGGPRCMSQPLFREDI

SEQ ID NO: 23 - protein, 417 aa, Pseudomonas plecoglossicida
MSAEKQKYGVHSEAGKLRKVMVCAPGLAHKRLTPSNCDELLFDDVIWVDQAKRDHFDFVTKMRERGVDVLEMHNLLTDIVQNPEALKWILDRKITPDTVGVGLTNEVRSWLEGQEPRHLAEFLIGGVAGQDLPESEGASVVKMYNDYLGHSSFILPPLPNTQFTRDTTCWIYGGVTLNPMYWPARRQETLLTTAIYKFHPEFTKADFQVWYGDPDQEHGQATLEGGDVMPIGKGIVLIGMGERTSRQAIGQLAQNLFAKGAVEQVIVAGLPKSRAAMHLDTVFSFCDRDLVTVFPEVVREIVPFIIRPDESKPYGMDVRRENKSFIEVVGEQLGVKLRVVETGGNSFAAEREQWDDGNNVVALEPGVVIGYDRNTYTNTLLRKAGIEVITISAGELGRGRGGGHCMTCPIVRDPINY

SEQ ID NO: 24 - protein, 417 aa, Pseudomonas putida
MSAEKQKYGVHSEAGKLRKVMVCAPGLAHKRLTPSNCDELLFDDVIWVDQAKRDHFDFVTKMRERGVDVLEMHNLLTDIVQNKDALKWILDRKITPDTVGVGLTNEVRSWLEGLEPRHLAEFLIGGVAGQDLPQSEGADVVKMYNDYLGHSSFILPPLPNTQFTRDTTCWIYGGVTLNPMYWPARRQETLLTTAIYKFHPQFTGADFQVWYGDPDKDHGNATLEGGDVMPIGKGIVLIGMGERTSRQAIGQLAQNLFAKGAVEKVIVAGLPKSRAAMHLDTVFSFCDRDLVTIFPEVVKEIVPFIIRPDESKPYGMDVRRENKSFIEVVGEQLGVKLRVVETGGNSFAAEREQWDDGNNVVAVEPGVVIGYDRNTYTNTLLRKAGIEVITISAGELGRGRGGGHCMTCPIVRDPIDY

SEQ ID NO: 25 - protein, 418 aa, Pseudomonas aeruginosa
MSTEKTKLGVHSEAGKLRKVMVCSPGLAHQRLTPSNCDELLFDDVIWVNQAKRDHFDFVTKMRERGIDVLEMHNLLTETIQNPEALKWILDRKITADSVGLGLTSELRSWLESLEPRKLAEYLIGGVAADDLPASEGANILKMYREYLGHSSFLLPPLPNTQFTRDTTCWIYGGVTLNPMYWPARRQETLLTTAIYKFHPEFANAEFEIWYGDPDKDHGSSTLEGGDVMPIGNGVVLIGMGERSSRQAIGQVAQSLFAKGAAERVIVAGLPKSRAAMHLDTVFSFCDRDLVTVFPEVVKEIVPFSLRPDASSPYGMSIRREEKTFLEVVAESLGLKKLRVVETGGNSFAAEREQWDDGNNVVCLEPGVVVGYDRNTYTNTLLRKAGVEVITISASELGRGRGGGHCMTCPIIRDPIDY

SEQ ID NO: 26 - protein, 402 aa, unidentified
  REGION 1..402: note = Mycobacterium tuberculosis complex
MGVELGSNSEVGALRVVILHRPGAELRRLTPRNTDQLLFDGLPWVSRAQDEHDEFAELLASRGAEVLLLSDLLTEALHHSGAARMQGIAAAVDAPRLGLPLAQELSAYLRSLDPGRLAHVLTAGMTFNELPSDTRTDVSLVLRMHHGGDFVIEPLPNLVFTRDSSIWIGPRVVIPSLALRARVREASLTDLIYAHHPRFTGVRRAYESRTAPVEGGDVLLLAPGVVAVGVGERTTPAGAEALARSLFDDDLAHTVLAVPIAQQRAQMHLDTVCTMVDTDTMVMYANVVDTLEAFTIQRTPDGVTIGDAAPFAEAAAKAMGIDKLRVIHTGMDPVVAEREQWDDGNNTLALAPGVVVAYERNVQTNARLQDAGIEVLTIAGSELGTGRGGPRCMSCPAARDPL

SEQ ID NO: 27 - protein, 409 aa, Mycoplasma arthritidis; identical to SEQ ID NO: 5

SEQ ID NO: 28 - protein, 410 aa, synthetic construct
  REGION 1..410: note = Artificial full length of Mycoplasma phocicerebrale
MSVFDSKFNGIHVYSEIGELETVLVHEPGREIDYITPARLDELLFSAILESHDARKEHQSFVKQLKDNGINVVELTDLVAETFDLASKEEQEKLIEEFLEDSEPVLSEAHKTAVRKFLTSRKSTREMVEFMMAGITKYDLGIEADHELIVDPMPNLYFTRDPFASVGNGVTIHYMRYKVRQRETLFSRFVFSNHPKLVKTPWYYDPAMKMSIEGGDVFIYNNDTLVVGVSERTDLETITLLAKNIKANKEVEFKRIVAINVPKWTNLMHLDTWLTMLDKDKFLYSPIANDVFKFWDYDLVNGGAEPQPKENGLPLEGLLQSIINKKPVLIPIAGNNASHIDIERETHFDGTNYLAIKPGVVIGYARNEKTNAALAAAGIKVLPFHGNQLSLGMGNARCMSMPLSRKDVKW

SEQ ID NO: 29 - protein, 410 aa, synthetic construct
  REGION 1..410: note = Artificial full length of Mycoplasma gateae
MSVFDSKFNGIHVYSEIGELESVLVHEPGREIDYITPARLDELLFSAILESHDARKEHKLFVSELKANDINVVELTDLVTETYDLASQEAKDNLIEEFLEDSEPVLTEELKSVVRTYLKSIKSTRELIQMMMAGITKYDLGIEADHELIVDPMPNLYFTRDPFASVGNGVTIHYMRYKVRQRETLFSRFVFSNHPKLVNTPWYYDPSLKLSIEGGDVFIYNNNTLVVGVSERTDLETVTLLAKNIVANKECEFKRIVAINVPKWTNLMHLDTWLTMLDKDKFLYSPIANDVFKFWDYDLVNGGEEPQPVENGLPLEGLLESIINKKPILIPIAGEGASQIDIERETHFDGTNYLAIRPGVVIGYSRNEKTNAALEAAGIKVLPFHGNQLSLGMGNARCMSMPLSRKDVKW

SEQ ID NO: 30 - protein, 410 aa, synthetic construct
  REGION 1..410: note = Artificial full length of Mycoplasma phocidae
MSVFDSKFNGIHVYSEIGELQTVLVHEPGREIEYITPARLDELLFSAILESHDARKEHQEFVAELKKNNINVVELTDLVSETYDMVSKEKQEKLIEEFLEDSEPVLSEEHKGLVRKFLKSLKSSKELIQYMMAGITKHDLNIEADHELIVDPMPNLYFTRDPFASVGNGVTIHYMRYKVRQRETLFSRFIFANHPKLMNTPLYYNPDMKLSIEGGDVFVYNNETLVVGVSERTDLDTITLLAKNIKANKEREFKRIVAINVPKWTNLMHLDTWLTMLDKDKFLYSPIANDVFKFWDYDLVNGGDEPQPKVNGLPLEKLLESIINKKPILIPIAGTSASNIDVERETHFDGTNYLAIAPGVVIGYSRNVKTNEALEAAGIKVLPFKGNQLSLGMGNARCMSMPLSRKDVKW

SEQ ID NO: 31 - protein, 410 aa, Mycoplasma salivarium
MSVFSSKFNGIHVYSEIGELETVLVHEPGKEIDYITPSRLDELLFSAILESHDARKEHQEFVATLKKEKINVVELTDLVTETYDLVDQKTKDKLIDEFLEDSEPVLTAELKATVKKFLKSFKETRKLIEVMMAGITKYDLGIKADRELIVDPMPNLYFTRDPFASVGNGVTIHYMRYKVRQRETLFSRFIFNNHPKLVKTPWYYDPAMKMSIEGGDVFIYNNDTLVVGVSERTDLDTITLLAKNIKANKECEFKRIVAINVPKWTNLMHLDTWLTMLDKDKFLYSPIANDIFKFWDYDLVNGGANPQPKDNGLPLDKLLKSIIGKEPVLIPIAGHHATEIEVARETHFDGTNYLAIRPGVVIGYARNEKTNEALKDAGITVLPFKGNQLSLGMGNARCMSMPLSRKDVKW

SEQ ID NO: 32 - protein, 411 aa, Mycoplasma spumans
MSVFDSKFKGIHVYSEIGELESVLVHEPGREIDYITPARLDELLFSAILESHDARKEHKGFVAELKKQNVNVIELTDLVAETYELASKEAQAKLIEDFIEDSEPVLNAEEAQAVRKFLSERKSTREMVEYMMSGLTKYELGLESADRELIVDPMPNLYFTRDPFASVGNGVTIHYMKYKVRQRETLFAKFVFSNHPKLVNTPRYYDPSMKLPIEGGDVFIYNNETLVVGCSERTELETITLLAKNIKANKEVEFKRIVAINVPKWTNLMHLDTWLTMLDKDKFLYSPIANDVFKFWDYDLVNGGEEPQPVENGLPLEELLASIINKKPTLIPIAGEGATHIDVERETHFDGTNYLAIAPALIIGYSRNEKTNAALEKAGITVLPFHGNQLSLGMGNARCMSMPLSRKDVKW

SEQ ID NO: 33 - protein, 410 aa, Mycoplasma auris
MSVFDSKFKGIHVYSEIGELETVLVHEPGREIDYITPKRLDELLFSAILESHEARKEHKQFVAELKANDINVVELTDLVAETYDLVSQELKDKLIEEFLDDSYPVLTEEHKKAVRSFLKSRSSTRELIEYMMAGITKYDLGIEAEGDLIVDPMPNLYFTRDPFASVGNGVTIHYMRYKVRQRETLFSRFIFDNHPKLVNTPRYYDPSLKLSIEGGDVFIYNNDTLVMGVSERTDLETVTLLAKNIVANKECEFKRIVAINVPHWTNLMHLDTWLTMLDKDKFLYSPIANDYFKFWDYDLVNGGAEPQPVVNELPLDKLLESIIHKKPILIPIAGEGASQIDLERETHFDGTNYLVLRPGVVVGYARNEKTNAALEAVGIKVLPFYGNQLSLGMGNSRCMSMPLSRKDVKW

SEQ ID NO: 34 - protein, 410 aa, Mycoplasma hyosynoviae
MSVFNSKFKGIHVYSEIGDLESVLVHEPGKEIDYITPSRLDELLFSAILESNDARKEHKEFVEILKKEGVNVVELVDLIAETIDLVDAKKKEALIDEYIEDSEPVVDAKVKPLVKKLLLGIKDTKELVKLMMAGITKYDLEIESEKELIIDPMPNLYFTRDPFASVGNGVTIHYMRYKVRQRETLFSRFVFRNHPKLTSTPWYYDPAMKLSIEGGDVFIYNNDTLVVGVSERTDLDTITLLAKNIKANKECEFKRIVAINVPKWTNLMHLDTWLTMLDKDKFLYSPIANDIFKFWDYDLVNGGSEPQPKDNGLPLEKLLESIIGKKPVLIPIAGCCASDIEIARETHFDGTNYLAIKPGVVIGYARNEKTNKALEKAGIKVLPFKGNQLSLGMGNARCMSMPLSRKDVKW

SEQ ID NO: 35 - protein, 409 aa, Mycoplasma cloacale
MSVFDKRFKGIHVYSEIGELQTVLVHEPGREIDYITPARLDELLFSAILESHDARKEHKEFVKILESQGINVVELTDLIAETYELASEEAKDNLIEEFLDESEPVLSEEHRILVRNFLKGITKTKELVKMMMAGITKYDLGIEADRELIVDPMPNLYFTRDPFASVGNGVTIHYMRYKVRQRETLFSRFIFENHPKLVSTPIYYHPSQGLSIEGGDVFIYNNDTLVVGVSERTDLQTITLLAKNIKANEECEFKRIVAINVPKWTNLMHLDTWLTMLDKNKFLYSPIANDVFKFWDYDLVNGGDEPQPVDNGLPLNELLASIIGEEPVLVPIAGEGASKMDIERETHFDGTNYLAIAPGVVVGYSRNEKTNAALEKAGIKVLPFKGHQLSLGMGNARCMSMPLYRKDVK

SEQ ID NO: 36 - protein, 412 aa, Mycoplasma alkalescens
MSVFDSKFKGIHVYSEIGELESVLVHEPGHEIDYITPSRLDELLFSAMLESHDARKEHKQFVAELKANNVNVIELTDLVAETYDLASQEAKDKLIEEFLEDSEPVLSEENKIAVRDFLKSRKTTRELIEVMMAGITKYDLGIKNCKCQDLVVDPMPNLYFTRDPFASVGNGITIHYMRYKVRQRETLFSRFIFANHPKLVNTPIYYHPSLKLSIEGGDVFIYNNDTLVVGVSERTDLETITLLAKNIVANKECEFKRIVAINVPKWTNLMHLDTWLTMLDKDKFLYSPIANDVFKFWDYDLVNGGAEPKPVENGSSLEAILESIIHKKPILIPIGGDSASQIEVERETHFDGTNYLAIRPGVVIGYSRNVKTNAALEAAGIKVIPFHGNQLSLGMGNARCMSMPLSRKDVKW

SEQ ID NO: 37 - protein, 401 aa, Mycoplasma iners
MSKINVYSEIGVLKEVLVHTPGDEIRRIAPSRLDELLFSAILEPSAAIQEHKSFLKILQDRGIKTIQLSDLVAETYKHYASEAEKEAFIEKYLDEATPVLSKDMRAKVKNYILSMQGEPVKMVRTMMAGVSKQELNVESEVELIVDPMPNLYFTRDPFASAGNGISLNNMKYVVRKRETIFAEFIFSIHPEYKKTPHWFDRLDNGSIEGGDVFIYNKDTLVIGVSERTNKEAIITIAKHIQDNKEAQFKKIVAINVPPMPNLMHLDTWLTMVDKNKFLYSPNMLSVLKVWEIDLSKPIEMVETNKPLAEVLESIIGEKPILIPIAGKDATQLDIDIETHFDGTNYLTIAPGVVVGYSRNVKTEAALRAAGVTVLSFEGNQLSLGMGSARCMSMPLVREDVK

SEQ ID NO: 38 - protein, 401 aa, Mycoplasma gallinarum
MSKIRVYSEIGNLKKVLVHTPGDEIRRISPSRLEELLFSAVLEPNAAIEEHKRFVKLLEDRGIQAIQLSDLVAETYVKYATAEQKAAFIEKYLDEATPALSAENRERAKKYILSLEMQPVKMIRTMMAGLSKYELNVESNIELIIDPMPNLYFTRDPFASAGNGISLNNMKYVVRKRETIFAEFIFAIHPEYKETPHWFDRLDHGSIEGGDVFVYNKDTLVIGVSERTNKEAIITIAKHIQDNKEAEFKKIVAINVPPMPNLMHLDTWLTMVDKNKFIYSPNMLSVLKIWEIDLAKPIEMVESNKSLTEVLESIIGEKPILIPIAGEGASQLDIDIETHFDGTNYLTIAPGVVVGYSRNEKTEKALKAAGITVLSFEGNQLSLGMGSARCMSMPLVREDVK

SEQ ID NO: 39 - protein, 405 aa, Mycoplasma pirum
MNSNQKGIHVYSEIGKLKEVLVHRPGRELDFLDPTRLDELLFAATLEAETARLEHDNFTNALKNQGVTVIELADLVAQTYSSSTPTIKAAFINKYLDEATPALTTKLRTLVKDFLTKQKSVRKMVDYMIGGILSTDLNIKGKPELIVEPMPNAYFTHDPFASVGNGVTLHYMKHNVRRREVLFSEFIFNNNERFQNTPRYIVPTKGLDIEGGDVFVYNKNTLVVGVSERTKMVTIKELAKNILKNKECLFKKIYAINVPKMPNLMHLDTWLTMLDHNKFLYSPNMLSVLKIWEIDISSGKSISSPKELNMDLSKALSIIIGKKPILIPVAGENASQIDINIETNFDATNYLVTQPGVVVGYSRNKKTEAALIKAGIEVIPFQGNQLSLGMGSARCMSMPLIREDV

SEQ ID NO: 40 - protein, 404 aa, Mycoplasma primatum
MSKSKINVYSEYGNLKEVLVHTPGDEIRRITPSRLDELLFSAILEPKSAIAEHKSFCQILKDNKVKAIQLDELVAATYKGVSESVQNSFVERWLDECEPKLENNVRPIVKEYLLKAAEQSVKKMIRIMMAGIDKREIGVESEVDFIVDPMPNLYFTRDPFASVGNGITLHHMKYVVRQRETLFSEFIFDNHPDYKFVPRYFDRDDEGKIEGGDVFIYNSKTLVVGISERTNKDAIRIVAKKIQANADAKFEKIFAINVPPMPNLMHLDTWLTMLDSNKFLYSPNMLSVLKVWEINLDDPALEWKEISGSLEEILTYIIGKKPILIPIAGKGASQFEIDIETHFDGTNYLAIAPSVVIGYSRNELTEKALKKAGVKVLSLDGNQLSLGMGSARCMSMPLIREDVK

SEQ ID NO: 41 - protein, 401 aa, Mycoplasma lipofaciens
MSKINVYSEVGVLKEVLVHTPGDEIRRVAPSRLDELLFSAILEPQDAIAEHKRFIKILEDNNIKVIQLDELVSETWEKATAEQRDAFIEKWLDEAEPVLDAKLRETVKKYLLSLNPVKKMVRTMMAGIDKKELKIELDRDLVVDPMPNLYFTRDPFASAGNGISLNNMKYVTRKRETIFAEFIFNIHPDYKTTPHWFDRLDKGNIEGGDVFIYNKDTLVLGVSERTNKDAVMTIAKHIQSNEQAKFKKLVAINVPPMPNLMHLDTWLTMVDHDKFLYSPNMLSVLKIWEIDLTPGKEIEMVESTKSLSDMLESIIGKKPVLIPIAGKDASQLDIDIETHFDGTNYLTIRPGVVVGYSRNCLTEQALKDAGVTVLSFDGNQLSLGMGSARCMSMPLVREDIK

SEQ ID NO: 42 - protein, 405 aa, Mycoplasma felifaucium
MNKINVYSEIGKLKEVLVHTPGNEIRRISPSRLDELLFSALLEPNFAAKEHTAFCEILKENGIKAIQLVDLVSDTWRIASEKAKTEFIERWLDECEPKLDSNLREIVRKHIYAIEKRSVKRMVKTMMAGIERRELPVTSKEVARELVVDPMPNLYFTRDPFASVGNGISLHHMKYVTRQRETIFAEFVFGNHPDYIDTPRWFDRSDDGRIEGGDVFIYGSKTLVIGVSERTNKEAIKVMAKKIQANKEATFEKIYAINVPPMPNLMHLDTWLTMLDKNKFLYSPNMLAVLQVWEIDLKDPELTWHELSGSLEEILHKIIGRKPILIPIAGHGAQQIDIDIETHFDGTNYLAIAPGVVVGYNRNVLTERALKKAGIKVLSFEGNQLSLGMGSARCMSMPLIRENLK

SEQ ID NO: 43 - protein, 404 aa, Mycoplasma imitans
MFNKIKVYSEIGRLRKVLVHTPGKELEYVTPQRLDELLFSSLLNPVKARQEHEAFIKILQDQGVECVQLTTLTAQTFQSATSEVKEKFINRWLDECLPKLSDDNRIKVYAYLKDLSSDPEVMIRKMMSGILAKEVNVQSDVELIADPMPNLYFTRDPFASIGKGVTLHSMFHPTRKRETIFADFVFSHHPEYKQTPKYYSRLNEYSIEGGDLFVYDDKTLVIGVSERTEKKAIQFLAEKLRENYETTFEKIYAINVPKMSNLMHLDTWLTMLDYDKFLYSPNMMGVLKIWEIDLTHEQLSWRELNESLEEFLSMVIGKKATTIPVAGEDSTQIEIDVETNFDATNFLVIQPGVVVGYDRNYKTNQALVNAGIKVLSWNGDQLSLGMGSARCMSMPLYRDPIKKG

SEQ ID NO: 44 - protein, 401 aa, Mycoplasma opalescens
MSKINVYSEIGTLKEVLVHTPGDEIRRVAPARLDELLFSAILEPNHAIAEHKAFIKILEDNGIKVIQLDELVVQTWNQVDEATRKAFVTKWLDECEPKLESNVRVEVEKYIYSLAKEPKKMVRTMMAGISKEELPLNVNRPLVVDPMPNLYFTRDPFASVGTGISLHHMKYVTRQRETIFAQFVFDNHKDYNTVPRWFDNKDQGRIEGGDVFIYNTKTLVIGVSERTDKDAIKIMAKKIQADKNCKFEKIFAINVPPMPNLMHLDTWLTMVDRNKFLYSPNMLSVLKVWEIDLKDASLAWKEIEGSLSQILEKIIGEKPILIPIAGENASQLDIDIETHFDGTNYLTIAPGVVVGYSRNVKTEQALKAAGVKVLSFEGNQLSLGMGSARCMSMPLIREDLK

SEQ ID NO: 45 - protein, 425 aa, Mycoplasma moatsii
MKKNAINVYSEIGKLKKVLVHRPGDELKYVTPQRMDELLMSAIIELEQAKEEHDAFTKILRDNGVEVIELADLTAEMYDSLTPSEKDAFLNQWVKEASWGKKSSIDALKIKKNLSKKVFDYVKSIKPTRKMIDKLMAGVLLSEIGEKSIILNKDKKNEMVIDLVVDPMPNLYFTRDPFASVGNGITLHNMKYPTRKRETIFAQWIFNKHPEYKDVPQFISKRDGKETIEGGDVFIYTKDVLAIGVSERTNMEAILRIATNIKKDKNCEFKKIVAINVPPMGNLMHLDTWLTMLDKDLFLYSGNIKSALKVWEIDLTKPITPKSPKLSTAKLADILAKIVGKKVRMIPIGGKDGNQMDIDIETHFDGTNYLAIAPGVVVGYHRNRKTQKALEEAGVKVLAFQGNQLSLGMGSARCMSMPLVREEVK

SEQ ID NO: 46 - protein, 399 aa, Mycoplasma elephantis
MSQINVFSEIGQLKEVLVHTPGDEIRRISPKRYNELLFSAILEADVAIKEHKSFVKILEENNVKVIQLKDILLETWNICSKEAKNIFINKWIEEAQPVIHSSSLKEKIKLFLKSKTPLEIIDIMMKGILKQELGIEYKHELIIDPMPNLYFTRDPFTSMGSGITINNMKYQTRKRETIFSEFIFNNHPKYKNTPRWFDRFDSGNIEGGDLFVYTKETIVVGVSERTKKKAILKIAKNIQENNNSFKKIVVIKVPIMQNLMHLDTWIVMVDFDKFIYSPNVTKSLKFWEIDLTKKPKFIQLKNETLEDVLYRVIGKKPILIPVAGENANQIDIDVETHFDATNYLTIRPGVVVGYSRNKKTEEALINAGVKVYAFEGNQLSLGMGSARCMSMPLIREDII

SEQ ID NO: 47 - protein, 399 aa, Mycoplasma testudinis
MKNINVYSEVGKLKEVVVHTPGEELHNVAPSRLQELLTSAVLEPEVARKEHLKFIKILNDYGVKVIQIVDLITETYEAVDSNKKEAFINNWLDNSVPKLTDKNRMILRNYLTQFSTKAMIRKMISGIRAKELNLKTPSALLVDPMPNLCFARDTFACVGSAISLSTMKHPTRRREALLTEFIFQNHPKYKDVIKYFDSKNSKATIEGGDIFVYNPKTLVVGNSERTNMQACLLLAKKIQSNPNNKFEKIVIVNVPPLPHLMHLDTWLTMVDYDKFIYSPNILHTLKFWVIDLKKRKLEAVEKHNTLKAMLRMIIKKEPILIPVGDVGADQLDIDLETHFDATNYLALAPGVVVGYDRNIKTQRALEKAGVKVLSFSGNQLSLAMGSARCLSMPLIREEN

SEQ ID NO: 48 - protein, 410 aa, Mycoplasma canadense
MSVFDSKFKGIHVYSEIGELESVLVHEPGREIDYITPARLDELLFSAILESHDARKEHKQFVSELKANDINVVELTDLVAETYDLASQEAKDKLIEEFLEDSEPVLSEEHKAIVRKYLKGIQPTRKLIEMMMAGITKYDLGIEADHELIVDPMPNLYFTRDPFASVGNGVTIHYMRYKVRQRETLFSRFVFSNHPKLVNTPWYYDPSLKLSIEGGDVFVYNNDTLVVGVSERTDLQTVTLLAKNIVANKECEFKRIVAINVPKWTNLMHLDTWLTMLDKDKFLYSPIANDVFKFWDYDLVNGGSEPQPVENGLPLEGLLESIINKKPILIPIAGEGASQMEIERETHFDGTNYLAIRPGVVIGYSRNEKTNAALEAAGIKVLPFHGNQLSLGMGNARCMSMPLSRKDVKW

SEQ ID NO: 49 - protein, 409 aa, Mycoplasma anseris
MSVFDKRFKGIHVYSEIGELQTVLVHEPGREIDYITPARLDELLFSAILESHDARAEHKKFVATLKEQGINTVELTDLVAETYDLASQEARDNLLEEFLDDSAPVLSEEHKEIVRTYLKGIKGTRKLIETMMAGITKYDLGIEAEQELIVDPMPNLYFTRDPFASVGNGVTIHYMRYKVRQRETLFSRFIFSNHPQLVNTPWYYNPAEGLSIEGGDVFIYNNDTLVVGVSERTDLQTITLLAKNIKANEECEFKRIVAINVPKWTNLMHLDTWLTMLDTNKFLYSPIANDVFKFWDYDLVNGGDEPQPVDNGLPLNELLKSIIGEEPILIPIAGDGATQIEIERETHFDGTNYLAIAPGVVIGYSRNEKTNAALEAAGIKVLPFKGHQLSLGMGNARCMSMPLYRKDVK

SEQ ID NO: 50 - protein, 401 aa, Mycoplasma meleagridis
MSKINVYSEIGVLKEVLVHTPGDEIRRISPSRLDELLFSAILQPEQAIKEHQSFVKILQDRGIKVIQLSDLVAETYVKYATSKEKESFIEKWLDEATPALNSENRARVKNYITAMQGQPVKMVRAMMAGVSKQELNIESDVELIVDPMPNLYFTRDPFASAGNGISLNNMKYVVRKRETIFAEFIFSIHPEYKQTPHWFDRLDKGNIEGGDVFIYNKDTLVIGVSERTNKEAILTIAEHIKNNKEAKFKKIVAINVPPMPNLMHLDTWLTMVDKNKFLYSPNMLSVLKIWEIDLSKEIKMVETSKPLADVLESIIGEKPILIPIAGENASQLDIDIETHFDGTNYLTIAPGVVVGYSRNVKTEAALKAAGVTVYSFDGNQLSLGMGSGRCMSMPLVREDVK

SEQ ID NO: 51 - protein, 404 aa, Mycoplasma alvi
MSIKENGIHVYSEIGKLRDVLVHRPGRELNFLDPSRLDELLFAATLEPETARLEHDNFTTVLKNQGVNVIELADLVSQTYSKVDSKVKKEFIDQYLNEATPKLTSELSKKVYDFLTKQKSNREMVDFMMGGILSSDLNIKGQPYLIVEPMPNLYFTRDPFASVGNGATIHWMKHNVRRREVLFANFIFKYNERFQNTPKYITPTKGLDIEGGDVFVYNKKTLVVGVSERTKMETIKELAKNISKNKECTFTKIYAINVPKMPNLMHLDTWLTMLDYNKFLYSPNMLSVLKVWEINISNNKVSAPKELNVNLEKALSMIIGKKPILIPVAGANASQIDINIETNFDATNYLVIEPGVVVGYSRNKKTEEALVKAGIKVLPFHGNQLSLGMGSARCMSMPLYREDV

SEQ ID NO: 52 - protein, 410 aa, Mycoplasma penetrans
MSSIDKNSLGNGINVYSEIGELKEVLVHTPGDEIRYTAPSRLEELLFSAVLKADTAIEEHKGFVKILQNNGIKVIQLCDLVAETYELCSKEVRNSFIEQYLDEALPVLKKEIRPVVKDYLLSFPTVQMVRKMMSGILANELNIKQDNPLIIDGMPNLYFTRDPFASMGNGVSINCMKYPTRKREVIFSRFVFTNNPKYKNTPRYFDIVGNNGTIEGGDIFIYNSKTLVIGNSERTNFAAIESVAKNIQANKDCTFERIVVINVPPMPNLMHLDTWLTMLDYDKFLYSPNMMNVLKIWEIDLNVKPVKFVEKKGTLEEVLYSIIDKKPILIPIAGKGANQLDIDIETHFDGTNYLTIAPGVVVGYERNEKTQKALVEAGIKVLSFNGSQLSLGMGSARCMSMPLIRENLKK

SEQ ID NO: 53 - protein, 404 aa, Mycoplasma fermentans
MKKINVYSEYGKLKEVLVHTPGDEIRRIAPSRLDELLFSAILEPDSAIAEHKRFVQLLKDNGIKVIQLDELFAKTFDLVSESVKQSFIERWLDECEPKLDATLRAKVKEYILELKAKSSKKMVRVMMAGIDKKELGIELDRDLVVDPMPNLYFTRDPFASVGNGISLHHMKYVTRQRETIFSEFIFDNNLDYNTVPRWFDRKDEGRIEGGDVFIYSADTLVVGVSERTNKEAINVMARKIAADKEVKFKRIYAINVPPMPNLMHLDTWLTMLDKNKFLYSPNMLSVLKVWRIDLNDPDFVWHEIEGSLEEILEQIIGMKPILIPIAGKGASQLDIDIETHFDGTNYLTIAPSVVVGYSRNEKTEKALKAAKVKVLSFEGNQLSLGMGSARCMSMPLIREDIKKK

SEQ ID NO: 54 - protein, 404 aa, Mycoplasma pneumoniae
MKYNINVHSEIGQLQTVLVHTPGNEIRRISPRRLDDLLFSAVIEPDTAIQEHQTFCQLLQEQNIEVVQLTDLTATTFDKANATAQNQFIETWLDQAEPKLTPEHRKVAKQYLLEQKAKSTLSMVRSMMGGIDKRKVAAANTINGDFLVDPMPNLYFTRDPFASIGHGISINRMKYLTRRRETLFASFIFANHPIIAARKFYFKPIDMGTIEGGDIFVYDQQTVVMGLSERTTEAAINVLAKKIQQDSSTSFKRIFVINVPQLPNLMHLDTWLTMLDRNKFLYSPNMLAVLKAWRIDFTDPALKWNEIAGDLSTILHTIIGQKPMLIPIAGADANQTEIDIETHFDGTNYLTIAPSVVVGYARNKLTHQTLEAAGVKVIAFKGNQLSLGMGSARCMSMPLVRKPL

SEQ ID NO: 55 - protein, 414 aa, Mycoplasma sp.
  REGION 1..414: note = Mycoplasma sp. CAG:877
MEKIHVTSEIGPLKKVLLHRPGNELLNLTPDTLSRLLFDDIPYLPDAIKEHDEFADALRANGVEVVYLENLMADVLDLSDEIRDKFIKQFIYEAGIRTPKYKYLVFDYLDQITNSKKLVLKTMEGIQISDIPRRKREIEKSLVDLIETEDEFIADPMPNLYFTRDPFASVGEGISLNKMYSVTRNRETIYAEYIFKYHPDYKDQARLYYDRYNPYHIEGGDVLNINDHVLAIGISQRTTAEAIDQIAKNLFKDPECKIDTILAFNIPESRAFMHLDTVFTQVDYDKFTYHPGIMGTLQVFEITEGDDPNSDEDLTVTEINAPLEEILTKYVGRKVTLIPCAGGDKVSAEREQWNDGSNTLCIAPGVVVVYDRNNLTNAVLRSYGLKVIEIHGAELSRGRGGPRCMSMPLVREDI

SEQ ID NO: 56 - protein, 408 aa, Mycoplasma sp.
  REGION 1..408: note = Mycoplasma sp. CAG:472
MHVTSEIKKLKKVLVHRPGKELLNLTPDTLGRLLFDDIPYLKDAILEHDEFCQILRDNDVEVVYLEDLMAETLDENPQVKPSFIRQFIYEAGVRTPKYKDLLFDYLMSYTNNKELVLKTMEGIKVSEVHRNKQDSEYSLVDQISEETKFLAEPMPNLYFTRDPFASVGDGIILNKMHSVTRSRETIYAYYIFNYHPDYMDKVPKYYDRENPFSIEGGDVLNLNEHTLAIGISQRTSAEAIDLVAKNMFNDEKCNIDTILAFKIPECRAFMHLDTVFTQIDIDKFTYHPGIMDTLEVFEITKNEDDLDEVRVIKKEGSLENILEEYLGIDITLIPCAGGDKIASEREQWNDGTNTLCIAPGVVVVYNRNNITNEVLREKGIKVIEMNSAELSRGRGGPRCMSMPLERED

(19) United States
(12) Patent Application Publication
(10) Pub. No.: US 2026/0216304 A1
(43) Pub. Date: Jul. 30, 2026

(54) FORMULATIONS OF PEGYLATED ARGININE DEIMINASE

(71) Applicant: POLARIS GROUP, Grand Cayman (KY)

(72) Inventors: Bor-Wen WU, San Diego, CA (US); James THOMSON, San Diego, CA (US); Wen-Chin TSAI, La Jolla, CA (US)

(21) Appl. No.: 19/247,311

(22) Filed: Jun. 24, 2025

Related U.S. Application Data
(63) Continuation of application No. 15/802,166, filed on Nov. 2, 2017, now Pat. No. 12,370,242.
(60) Provisional application No. 62/416,607, filed on Nov. 2, 2016.

(51) Int. Cl. A61K 38/50; A61K 9/00; A61K 47/60; C12N 9/78; C12N 9/96

(52) U.S. Cl. CPC A61K 38/50; A61K 9/0019; A61K 47/60; C12N 9/78; C12N 9/96; C12Y 305/03006

(57) ABSTRACT

The present disclosure relates to lyophilized formulations comprising pegylated arginine deiminase (ADI-PEG) and related reconstituted liquid compositions and methods of using the compositions for arginine depletion therapies, including for treatment of various cancers.

### CROSS-REFERENCE TO RELATED APPLICATIONS

This application is a continuation application of Ser. No. 15/802,166 filed Nov. 2, 2017 which claims priority under 35 U.S.C. § 119(e) to U.S. Application No. 62/416,607, filed Nov. 2, 2016, which are incorporated by reference in their entirety.

### STATEMENT REGARDING THE SEQUENCE LISTING

The Sequence Listing associated with this application is provided in XLM format in lieu of a paper copy, is part of the specification, and is hereby incorporated by reference in its entirety into the specification. The name of the XLM file containing the Sequence Listing is 2844-3CON.xlm. The XLM file is 76,564 bytes in size, was created on Mar. 31, 2026, and is being submitted electronically via EFS-Web.

### BACKGROUND

### Technical Field

Embodiments of the present disclosure relate, inter alia, to lyophilized formulations comprising pegylated arginine deiminase (ADI-PEG) and related reconstituted liquid compositions and methods of using the compositions for arginine depletion therapies, including for the treatment of various cancers.

### Description of the Related Art

Arginine depletion therapy can be an effective treatment of certain forms of cancer, among other diseases. For instance, pegylated arginine deiminase (ADI-PEG) can be used to deplete the bloodstream supply of arginine by converting it to citrulline and ammonia. ADI-PEG 20 is an exemplary ADI-PEG that is being investigated in the clinic for tumors deficient in the key enzyme argininosuccinate synthetase-1 (ASS 1), which is involved in the conversion of citrulline to arginine. ADI-PEG 20 has been well-tolerated and showed promise in clinical studies (see, e.g., Qiu et al., Cancer Lett. 2015 Aug. 1; 364(1):1-7; Phillips et al., Cancer Res Treat. 2013 December; 45(4):251-62; Feun et al., Curr Pharm Des. 2008; 14(11):1049-57; Feun and Savaraj, Expert Opin Investig Drugs. 2006 July; 15(7):815-22; Feun et al., Curr Opin Clin Nutr Metab Care. 2015 January; 18(1):78-82).

Lyophilization, or freeze drying, is a process that removes water from a liquid agent to create a solid powder, or cake. The preparation of lyophilized solid state or formulations represents one approach to optimizing the stability of biopharmaceutical agents. The use of lyophilized formulations can reduce degradative reactions, agitation during transportation, and the effects of temperature fluctuation during storage, among other benefits.

### BRIEF SUMMARY

Certain embodiments relate to lyophilized formulations, comprising a pegylated arginine deiminase (ADI-PEG), wherein the lyophilized formulation is sterile, substantially endotoxin-free, and at a pharmaceutically-acceptable pH.

Some embodiments comprise a pharmaceutically-acceptable buffer, for example, a buffer selected from one or more of histidine, sodium citrate, glycyl-glycine, sodium phosphate, Tris, and lysine. In certain embodiments, the buffer is at a concentration of about 0.10 mM to about 200 mM, or about 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1.0, 1.5, 2.0, 2.5, 3.0, 3.5, 4.0, 4.5, 5.0, 5.5, 6.0, 6.5. 7.0, 7.5, 8.0, 8.5, 9.0, 9.5, 10, 11, 12, 13, 14, 15, 16, 17, 18, 19, 20, 25, 30, 35, 40, 45, 50, 55, 60, 65, 70, 75, 80, 85, 90, 95, 100, 105, 110, 120, 125, 130, 135, 140, 145, 150, 155, 160, 165, 170, 175, 180, 185, 190, 195, or 200 mM, including all integers and ranges in between. In particular embodiments, the buffer is at about 1 to about 50 mM, or about 10 to about 30 mM, or about 15 to about 25 mM, or about 20 mM, or about 10 mM.

Some embodiments comprise a pharmaceutically-acceptable excipient, for example, an excipient selected from one or more of a cryoprotectant, a lyoprotectant, a stabilizer, a bulking agent, a tonicity modifier, a surfactant, a pharmaceutical plasticizer, a chelator, and any combination of the foregoing.

In some embodiments, the cryoprotectant is present at about 0.001% to about 20% (wt %), including all integers and ranges in between. In some embodiments, the cryoprotectant is selected from one or more of sucrose, trehalose, ethylene glycol, propylene glycol, glycerol, and any combination of the foregoing.

In some embodiments, the lyoprotectant is present at about 0.001% to about 20% (wt %), including all integers and ranges in between. In some embodiments, the lyoprotectant is selected from one or more of sucrose, trehalose, mannitol, sorbitol, glycerol, and any combination of the foregoing.

In some embodiments, the stabilizer is present at about 0.001% to about 20% (wt %), including all integers and ranges in between. In certain embodiments, the stabilizer is selected from one or more of sucrose, mannitol, lactose, trehalose, maltose, sorbitol, gelatin, albumin, and any combination of the foregoing.

In certain embodiments, the bulking agent is present at about 0.001% to about 20% (wt %), including all integers and ranges in between. In certain embodiments, the bulking agent is selected from one or more of mannitol, sorbitol, lactose, glucose, sucrose, glycine, albumin, dextran 40.

In certain embodiments, the tonicity modifier is present at about 0.001% to about 20% (wt %), including all integers and ranges in between. In particular embodiments, the tonicity modifier is selected from one or more of sodium chloride, sucrose, mannitol, and any combination of the foregoing.

Particular lyophilized formulations comprise a pharmaceutically-acceptable excipient selected from one or more of sucrose, trehalose, dextran, mannitol, proline, glycine, a surfactant, a pharmaceutical plasticizer, a chelator, and any combination of the foregoing.

Certain lyophilized formulations comprise sucrose at about 0.001% to about 20% (wt %), or about 0.001, 0.002, 0.003, 0.004, 0.005, 0.006, 0.007, 0.008, 0.009, 0.010, 0.015, 0.020, 0.025, 0.030, 0.035, 0.040, 0.045, 0.050, 0.055, 0.060, 0.065, 0.070, 0.075, 0.080, 0.085, 0.090, 0.095, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1.0, 1.1, 1.2, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 5.0, 5.1, 5.2, 5.3, 5.4, 5.5, 5.6, 5.7, 5.8, 5.9, 6.0, 6.1. 6.2. 6.3. 6.4. 6.5. 6.6. 6.7. 6.8. 6.9. 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10, 11, 12, 13, 14, 15, 16, 17, 18, 19, or 20%, including all integers and ranges in between.

Some lyophilized formulations comprise trehalose at about 0.001% to about 20% (wt %), or about 0.001, 0.002, 0.003, 0.004, 0.005, 0.006, 0.007, 0.008, 0.009, 0.010, 0.015, 0.020, 0.025, 0.030, 0.035, 0.040, 0.045, 0.050, 0.055, 0.060, 0.065, 0.070, 0.075, 0.080, 0.085, 0.090, 0.095, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1.0, 1.1, 1.2, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 5.0, 5.1, 5.2, 5.3, 5.4, 5.5, 5.6, 5.7, 5.8, 5.9, 6.0, 6.1. 6.2. 6.3. 6.4. 6.5. 6.6. 6.7. 6.8. 6.9. 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10, 11, 12, 13, 14, 15, 16, 17, 18, 19, or 20%, including all integers and ranges in between.

Certain lyophilized formulations comprise dextran, optionally dextran 40, at about 0.001% to about 20% (wt %), or about 0.001, 0.002, 0.003, 0.004, 0.005, 0.006, 0.007, 0.008, 0.009, 0.010, 0.015, 0.020, 0.025, 0.030, 0.035, 0.040, 0.045, 0.050, 0.055, 0.060, 0.065, 0.070, 0.075, 0.080, 0.085, 0.090, 0.095, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1.0, 1.1, 1.2, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 5.0, 5.1, 5.2, 5.3, 5.4, 5.5, 5.6, 5.7, 5.8, 5.9, 6.0, 6.1. 6.2. 6.3. 6.4. 6.5. 6.6. 6.7. 6.8. 6.9. 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10, 11, 12, 13, 14, 15, 16, 17, 18, 19, or 20%, including all integers and ranges in between.

Particular lyophilized formulations comprise mannitol at about 0.001% to about 20% (wt %), or about 0.001, 0.002, 0.003, 0.004, 0.005, 0.006, 0.007, 0.008, 0.009, 0.010, 0.015, 0.020, 0.025, 0.030, 0.035, 0.040, 0.045, 0.050, 0.055, 0.060, 0.065, 0.070, 0.075, 0.080, 0.085, 0.090, 0.095, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1.0, 1.1, 1.2, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 5.0, 5.1, 5.2, 5.3, 5.4, 5.5, 5.6, 5.7, 5.8, 5.9, 6.0, 6.1. 6.2. 6.3. 6.4. 6.5. 6.6. 6.7. 6.8. 6.9. 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10, 11, 12, 13, 14, 15, 16, 17, 18, 19, or 20%, including all integers and ranges in between.

Some lyophilized formulations comprise proline at about 0.10 mM to about 200 mM, or about 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1.0, 1.5, 2.0, 2.5, 3.0, 3.5, 4.0, 4.5, 5.0, 5.5, 6.0, 6.5. 7.0, 7.5, 8.0, 8.5, 9.0, 9.5, 10, 11, 12, 13, 14, 15, 16, 17, 18, 19, 20, 25, 30, 35, 40, 45, 50, 55, 60, 65, 70, 75, 80, 85, 90, 95, 100, 105, 110, 120, 125, 130, 135, 140, 145, 150, 155, 160, 165, 170, 175, 180, 185, 190, 195, or 200 mM, or at about 0.001% to about 20%, or about 0.001, 0.002, 0.003, 0.004, 0.005, 0.006, 0.007, 0.008, 0.009, 0.010, 0.015, 0.020, 0.025, 0.030, 0.035, 0.040, 0.045, 0.050, 0.055, 0.060, 0.065, 0.070, 0.075, 0.080, 0.085, 0.090, 0.095, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1.0, 1.1, 1.2, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 5.0, 5.1, 5.2, 5.3, 5.4, 5.5, 5.6, 5.7, 5.8, 5.9, 6.0, 6.1. 6.2. 6.3. 6.4. 6.5. 6.6. 6.7. 6.8. 6.9. 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10, 11, 12, 13, 14, 15, 16, 17, 18, 19, or 20%, including all integers and ranges in between.

Certain lyophilized formulations comprise glycine at about 0.10 mM to about 200 mM, or about 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1.0, 1.5, 2.0, 2.5, 3.0, 3.5, 4.0, 4.5, 5.0, 5.5, 6.0, 6.5. 7.0, 7.5, 8.0, 8.5, 9.0, 9.5, 10, 11, 12, 13, 14, 15, 16, 17, 18, 19, 20, 25, 30, 35, 40, 45, 50, 55, 60, 65, 70, 75, 80, 85, 90, 95, 100, 105, 110, 120, 125, 130, 135, 140, 145, 150, 155, 160, 165, 170, 175, 180, 185, 190, 195, or 200 mM, or at about 0.001% to about 20% (wt %), or about 0.001, 0.002, 0.003, 0.004, 0.005, 0.006, 0.007, 0.008, 0.009, 0.010, 0.015, 0.020, 0.025, 0.030, 0.035, 0.040, 0.045, 0.050, 0.055, 0.060, 0.065, 0.070, 0.075, 0.080, 0.085, 0.090, 0.095, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1.0, 1.1, 1.2, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 5.0, 5.1, 5.2, 5.3, 5.4, 5.5, 5.6, 5.7, 5.8, 5.9, 6.0, 6.1. 6.2. 6.3. 6.4. 6.5. 6.6. 6.7. 6.8. 6.9. 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10, 11, 12, 13, 14, 15, 16, 17, 18, 19, or 20%, including all integers and ranges in between.

Certain lyophilized formulations comprise a surfactant at about 0.001% to about 20% (wt %), or about 0.001, 0.002, 0.003, 0.004, 0.005, 0.006, 0.007, 0.008, 0.009, 0.010, 0.015, 0.020, 0.025, 0.030, 0.035, 0.040, 0.045, 0.050, 0.055, 0.060, 0.065, 0.070, 0.075, 0.080, 0.085, 0.090, 0.095, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1.0, 1.1, 1.2, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 5.0, 5.1, 5.2, 5.3, 5.4, 5.5, 5.6, 5.7, 5.8, 5.9, 6.0, 6.1. 6.2. 6.3. 6.4. 6.5. 6.6. 6.7. 6.8. 6.9. 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10, 11, 12, 13, 14, 15, 16, 17, 18, 19, or 20%, including all integers and ranges in between. In some embodiments, the surfactant is selected from one or more of Tween-80, polysorbate 20 (P20), polysorbate 80 (P80), poloxamer 188, and any combination of the foregoing.

Some lyophilized formulations comprise a pharmaceutical plasticizer at about 0.001% to about 20% (wt %), or about 0.001, 0.002, 0.003, 0.004, 0.005, 0.006, 0.007, 0.008, 0.009, 0.010, 0.015, 0.020, 0.025, 0.030, 0.035, 0.040, 0.045, 0.050, 0.055, 0.060, 0.065, 0.070, 0.075, 0.080, 0.085, 0.090, 0.095, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1.0, 1.1, 1.2, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 5.0, 5.1, 5.2, 5.3, 5.4, 5.5, 5.6, 5.7, 5.8, 5.9, 6.0, 6.1. 6.2. 6.3. 6.4. 6.5. 6.6. 6.7. 6.8. 6.9. 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10, 11, 12, 13, 14, 15, 16, 17, 18, 19, or 20%, including all integers and ranges in between. In certain embodiments, the pharmaceutical plasticizer is glycerol.

Certain lyophilized formulations comprise a chelator, for example, ethylenediaminetetraacetic acid (EDTA). In some embodiments, the chelator is present at about 0.001% to about 1% (wt %), or about 0.001, 0.002, 0.003, 0.004, 0.005, 0.006, 0.007, 0.008, 0.009, 0.010, 0.015, 0.020, 0.025, 0.030, 0.035, 0.040, 0.045, 0.050, 0.055, 0.060, 0.065, 0.070, 0.075, 0.080, 0.085, 0.090, 0.095, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, or 1.0%, including all integers and ranges in between.

In certain embodiments, the pharmaceutically-acceptable pH is about 5.0 to about 8.0 (±0.01 to +0.1), or about 5.0, 5.1, 5.2, 5.3, 5.4, 5.5, 5.6, 5.7, 5.8, 5.9, 6.0, 6.1. 6.2. 6.3. 6.4. 6.5. 6.6. 6.7. 6.8. 6.9. 7.0, 7.1, 7.2, 7.3, 7.4, 7.5, 7.6, 7.7, 7.8, 7.9, or 8.0 (±0.01 to +0.1), including all integers and ranges in between.

In specific embodiments, the buffer is histidine at a concentration of about 1 to about 50 mM, or about 10 to about 30 mM, or about 15 to about 25 mM, or about 20 mM.

Certain of these and related embodiments comprise sucrose at about 1% to about 10%, or about 4% to about 6%, or about 5%. Some embodiments comprise mannitol at about 1% to about 10%, or about 4% to about 6%, or about 5%. Some embodiments comprise dextran, for example, dextran 40, at about 0.1% to about 5%, or about 0.5% to about 2%, or about 1%. Some embodiments comprise dextran, for example, dextran 40, at about 0.1% to about 5%, or about 0.5% to about 2%, or about 1%, and comprising sucrose at about 1% to about 10%, or about 4% to about 6%, or about 5%.

Some embodiments comprise trehalose at about 1% to about 10%, or about 4% to about 6%, or about 5%. Some embodiments comprise mannitol at about 1% to about 10%, or about 3% to about 5%, or about 4%, and comprising sucrose at about 0.1% to about 5%, or about 0.5% to about 2%, or about 1%. Some embodiments comprise sucrose at about 1% to about 10%, or about 4% to about 6%, or about 4.8% or 5%, and comprising Tween-80 at about 0.001% to about 0.1%, or about 0.005% to about 0.05%, or about 0.01%. Some embodiments comprise sucrose at about 1% to about 10%, or about 4% to about 6%, or about 4.8% or 5%, comprising Tween-80 at about 0.001% to about 0.1%, or about 0.005% to about 0.05%, or about 0.01%, and comprising glycerol at about 0.01% to about 1.0%, or about 0.1% to about 0.5%, or about 0.25%. In some of these and related embodiments, the pH is about 6.0 to about 6.5 to about 7.2 (±0.1), or at about 6.6 to about 7.0 (±0.1), or about 6.8 (±0.1).

Some embodiments comprise trehalose at about 1% to about 15%, or about 5% to about 12%, or about 10% or 9.5%. Some embodiments comprise trehalose at about 1% to about 15%, or about 5% to about 12%, or about 10% or 9.5%, and comprising proline at about 10 mM to about 40 mM, or about 15 to about 30 mM, or about 20 mM. Some embodiments comprise trehalose at about 5% to about 15%, or about 8% to about 12%, or about 10% or 9.5%, and comprising glycine at about 10 mM to about 40 mM, or about 15 to about 30 mM, or about 20 mM. Some embodiments comprise sucrose at about 1% to about 15%, or about 5% to about 12%, or about 10% or 9.5%. Some embodiments comprise sucrose at about 1% to about 10%, about 4% to about 6%, or about 5% or 4.5%, and comprising trehalose at about 1% to about 10%, about 4% to about 6%, or about 5% or 4.5%. Some embodiments comprise trehalose at about 5% to about 15%, or about 8% to about 12%, or about 10% or 9.5%, and comprising EDTA at about 0.01% to about 0.1%, or about 0.02% to about 0.08%, or about 0.05%. In some of these and related embodiments, the pH is about 6.0 to about 7.2 (±0.1), or at about 6.4 to about 6.8 (±0.1), or about 6.5 (±0.1).

In certain embodiments, the buffer is histidine at about 1 to about 30 mM, or about 5 to about 20 mM, or about 10 mM, comprising glycine at about 1% to about 5%, or about 2% or 1.9%, comprising trehalose at about 0.1% to about 2%, or about 0.5% to about 1.5%, or about 1%, and comprising a surfactant, for example, P20, at about 0.001% to about 0.1%, or about 0.005% to about 0.02%, or about 0.01%. In certain embodiments, the buffer is histidine at about 1 to about 30 mM, or about 5 to about 20 mM, or about 10 mM, comprising sucrose at about 1% to about 15%, or about 5% to about 12%, or about 10% or 8.5%, and comprising trehalose at about 0.1% to about 2%, or about 0.5% to about 1.5%, or about 1%. In certain embodiments, the buffer is histidine at about 1 to about 30 mM, or about 5 to about 20 mM, or about 10 mM, comprising sucrose at about 1% to about 15%, or about 5% to about 12%, or about 10% or 9%. In certain embodiments, the buffer is histidine at about 1 to about 50 mM, or about 10 to about 40 mM, or about 20, 25, 30, or 35 mM, comprising sodium chloride at about 100 to about 150 mM, or about 120, 130, or 140 mM. In some embodiments, the buffer is sodium phosphate at about 100 to about 200 mM, or about 150 mM, comprising sucrose and/or trehalose at about 1% to about 10%, about 4% to about 6%, or about 5% or 4.5%, comprising glycine at about 0.1 to about 1%, or about 0.5%, comprising glycerol at about 0.1 to about 1.0%, or about 0.25%, comprising Tween 80 at about 0.001 to about 0.1%, or about 0.01%, and comprising EDTA at about 0.01 to about 0.1%, or about 0.05%. In certain embodiments, the pH is about 6.0 to about 6.5 to about 7.2 (±0.1), or at about 6.6 to about 7.0 (±0.1), or about 6.8 (±0.1).

In some embodiments, the dry weight of the ADI-PEG is about 50 mg/g to about 150 mg/g. In some embodiments, the ADI-PEG comprises an amino acid sequence that is at least 80, 95, 90, 95, 96, 97, 98, 99, or 100% identical to a sequence in Table A1. In some embodiments, the ADI-PEG is covalently bonded to about 1 to about 21 PEG molecules. In some embodiments, the ADI-PEG comprises one or more water-labile linkers which covalently attach the ADI and PEG. In some embodiments, the ADI-PEG is ADI-PEG 20, wherein the arginine deiminase is covalently bonded to a mass average of about 5±1.0 PEG molecules.

In some embodiments, the ADI-PEG retains at least 80, 85, 90, or 95% of its arginine deiminase (ADI) activity relative to a corresponding ADI-PEG in a non-lyophilized liquid control composition. In some embodiments, the ADI-PEG retains at least 50, 55, 60, 65, 70, 75, 80, 85, 90, 95, or 100% of the (original) PEG molecules (per ADI monomer/protomer), for example, relative to a corresponding ADI-PEG in a non-lyophilized liquid control composition.

Also included are methods of reconstituting a lyophilized formulation described herein, comprising adding a pharmaceutically-acceptable solvent to the lyophilized formulation to form a reconstituted liquid composition.

In some embodiments, the lyophilized formulation is reconstituted to a substantially aggregate-free solution of about 5-20 mg/ml ADI-PEG in a time of less than about five minutes. In some embodiments, the lyophilized formulation is reconstituted to a substantially aggregate-free solution of about 5-20 mg/ml ADI-PEG in a time of less than about one or two minutes.

In some embodiments, the ADI-PEG in the reconstituted liquid composition retains at least 75, 80, 85, 90, or 95% of its arginine deiminase activity, and/or retains at least 50, 55, 60, 65, 70, 75, 80, 85, 90, 95, or 100% of the PEG molecules (per ADI monomer/protomer), relative to a corresponding ADI-PEG in a non-lyophilized liquid control composition. In some embodiments, the ADI-PEG retains at least 75, 80, 85, 90, or 95% of its arginine deiminase activity upon reconstitution after being stored as a lyophilized formulation for about or at least about 1, 2, 3, 4, 5, 6, 7, 8, 9, 10, 11, 12, 13, 14, 15, 16, 17, 18, 19, 20, 21, 22, 23, 24, 36, 48, 60, or 72 months, for example, after being stored at a temperature of about 2-8° C. and/or about room temperature. In some embodiments, the ADI-PEG retains at least 50, 55, 60, 65, 70, 75, 80, 85, 90, 95, or 100% of the (original) PEG molecules (per ADI monomer/protomer) after being stored as a lyophilized formulation for about or at least about 1, 2, 3, 4, 5, 6, 7, 8, 9, 10, 11, 12, 13, 14, 15, 16, 17, 18, 19, 20, 21, 22, 23, 24, 36, 48, 60, or 72 months, for example, after being stored at a temperature of about 2-8° C. and/or about room temperature. In some embodiments, the specific enzyme activity of the ADI-PEG after reconstitution is about 5.0 to about 120 IU/mg, or about 5.5, 6, 6.5, 7, 7.5, 8, 8.5, 9.0, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 35, 40, 45, 50, 55, 55, 60, 65, 70, 75, 80, 85, 90, 95, 100, 105, 110, 115, or 120 IU/mg.

In some embodiments, the ADI-PEG has an osmolality of about 50 mOsm/kg to about 500 mOsm/kg, or about 50, 55, 60, 65, 70, 75, 80, 85, 90, 95, 100, 105, 110, 120, 125, 130, 135, 140, 145, 150, 155, 160, 165, 170, 175, 180, 185, 190, 195, 200, 205, 210, 220, 225, 230, 235, 240, 245, 250, 255, 260, 265, 270, 275, 280, 285, 290, 295, 300, 305, 310, 320, 325, 330, 335, 340, 345, 350, 355, 360, 365, 370, 375, 380, 385, 390, 395, 400, 405, 410, 420, 425, 430, 435, 440, 445, 450, 455, 460, 465, 470, 475, 480, 485, 490, 495, or about 500 mOsm/kg.

In specific embodiments, the solvent is water.

Certain embodiments relate to reconstituted liquid compositions prepared by any of the methods described herein.

Some embodiments include a reconstituted liquid composition, comprising a lyophilized formulation described herein and a pharmaceutically-acceptable solvent. In some embodiments, the ADI-PEG is at a concentration of about 5-20 mg/ml, or about 5, 6, 7, 8, 9, 10, 10.5, 11, 11.5, 12, 12.5, 13, 13.5, 14, 15, 16, 17, 18, 19, or 20 mg/ml, including all integers and ranges in between. In some embodiments, the ADI-PEG in the reconstituted liquid composition retains at least 75, 80, 85, 90, or 95% of its arginine deiminase activity relative to a corresponding ADI-PEG in a non-lyophilized liquid control composition.

In some embodiments, the specific enzyme activity of the ADI-PEG is about 5.0 to about 120 IU/mg, or about 5.5, 6, 6.5, 7, 7.5, 8, 8.5, 9.0, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 35, 40, 45, 50, 55, 55, 60, 65, 70, 75, 80, 85, 90, 95, 100, 105, 110, 115, or 120 IU/mg.

In some embodiments, the ADI-PEG has an osmolality of about 50 mOsm/kg to about 500 mOsm/kg, or about 50, 55, 60, 65, 70, 75, 80, 85, 90, 95, 100, 105, 110, 120, 125, 130, 135, 140, 145, 150, 155, 160, 165, 170, 175, 180, 185, 190, 195, 200, 205, 210, 220, 225, 230, 235, 240, 245, 250, 255, 260, 265, 270, 275, 280, 285, 290, 295, 300, 305, 310, 320, 325, 330, 335, 340, 345, 350, 355, 360, 365, 370, 375, 380, 385, 390, 395, 400, 405, 410, 420, 425, 430, 435, 440, 445, 450, 455, 460, 465, 470, 475, 480, 485, 490, 495, or about 500 mOsm/kg.

In some embodiments, the solvent is water. Certain reconstituted solutions are suitable for injection into a subject.

Also included are methods of treating, ameliorating the symptoms of, or inhibiting the progression of, a cancer in a subject in need thereof, comprising administering to the subject a reconstituted liquid formulation described herein.

In certain embodiments, the cancer is selected from one or more of hepatocellular carcinoma (HCC), melanoma, metastatic melanoma, pancreatic cancer, prostate cancer, small cell lung cancer, mesothelioma, lymphocytic leukemia, chronic myelogenous leukemia, lymphoma, hepatoma, sarcoma, leukemia, acute myeloid leukemia, relapsed acute myeloid leukemia, B-cell malignancy, breast cancer, ovarian cancer, colorectal cancer, gastric cancer, glioma (e.g., astrocytoma, oligodendroglioma, ependymoma, or a choroid plexus papilloma), glioblastoma multiforme (e.g., giant cell gliobastoma or a gliosarcoma), meningioma, pituitary adenoma, vestibular schwannoma, primary CNS lymphoma, primitive neuroectodermal tumor (medulloblastoma), non-small cell lung cancer (NSCLC), kidney cancer, bladder cancer, uterine cancer, esophageal cancer, brain cancer, head and neck cancers, cervical cancer, testicular cancer, and stomach cancer.

In some embodiments, the cancer exhibits reduced expression of argininosuccinate synthetase-1.

Certain embodiments include one or more patient care kits, comprising a lyophilized formulation described herein, and optionally a pharmaceutically-acceptable solvent. In specific embodiments, the solvent is water.

### BRIEF DESCRIPTION OF THE DRAWINGS

FIGS. 1A-1D illustrate a variety of cysteine-reactive PEG molecules that can be conjugated to the ADI polypeptides described herein.

### DETAILED DESCRIPTION

Unless defined otherwise, all technical and scientific terms used herein have the same meaning as commonly understood by those of ordinary skill in the art to which the disclosure belongs. Although any methods, materials, compositions, reagents, cells, similar or equivalent similar or equivalent to those described herein can be used in the practice or testing of the subject matter of the present disclosure, preferred methods and materials are described. All publications and references, including but not limited to patents and patent applications, cited in this specification are herein incorporated by reference in their entirety as if each individual publication or reference were specifically and individually indicated to be incorporated by reference herein as being fully set forth. Any patent application to which this application claims priority is also incorporated by reference herein in its entirety in the manner described above for publications and references.

The practice of the present disclosure will employ, unless indicated specifically to the contrary, conventional methods of virology, immunology, microbiology, molecular biology and recombinant DNA techniques within the skill of the art, many of which are described below for the purpose of illustration. Such techniques are explained fully in the literature. See, e.g., Current Protocols in Protein Science, Current Protocols in Molecular Biology or Current Protocols in Immunology, John Wiley & Sons, New York, N.Y. (2009); Ausubel et al., Short Protocols in Molecular Biology, 3^{rd} ed., Wiley & Sons, 1995; Sambrook and Russell, Molecular Cloning: A Laboratory Manual (3rd Edition, 2001); Maniatis et al. Molecular Cloning: A Laboratory Manual (1982); DNA Cloning: A Practical Approach, vol. I & II (D. Glover, ed.); Oligonucleotide Synthesis (N. Gait, ed., 1984); Nucleic Acid Hybridization (B. Hames & S. Higgins, eds., 1985); Transcription and Translation (B. Hames & S. Higgins, eds., 1984); Animal Cell Culture (R. Freshney, ed., 1986); Perbal, A Practical Guide to Molecular Cloning (1984) and other like references.

Standard techniques may be used for recombinant DNA, oligonucleotide synthesis, and tissue culture and transformation (e.g., electroporation, lipofection). Enzymatic reactions and purification techniques may be performed according to manufacturer's specifications or as commonly accomplished in the art or as described herein. These and related techniques and procedures may be generally performed according to conventional methods well known in the art and as described in various general and more specific references that are cited and discussed throughout the present specification. Unless specific definitions are provided, the nomenclature utilized in connection with, and the laboratory procedures and techniques of, molecular biology, analytical chemistry, synthetic organic chemistry, and medicinal and pharmaceutical chemistry described herein are those well-known and commonly used in the art. Standard techniques may be used for recombinant technology, molecular biological, microbiological, chemical syntheses, chemical analyses, pharmaceutical preparation, formulation, and delivery, and treatment of patients.

For the purposes of the present disclosure, the following terms are defined below.

The articles “a” and “an” are used herein to refer to one or to more than one (i.e., to at least one) of the grammatical object of the article. By way of example, “an element” means one element or more than one element.

By “about” is meant a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length.

As used herein, the term “amino acid” is intended to mean both naturally occurring and non-naturally occurring amino acids as well as amino acid analogs and mimetics. Naturally occurring amino acids include the 20 (L)-amino acids utilized during protein biosynthesis as well as others such as 4-hydroxyproline, hydroxylysine, desmosine, isodesmosine, homocysteine, citrulline and ornithine, for example. Non-naturally occurring amino acids include, for example, (D)-amino acids, norleucine, norvaline, p-fluorophenylalanine, ethionine and the like, which are known to a person skilled in the art. Amino acid analogs include modified forms of naturally and non-naturally occurring amino acids. Such modifications can include, for example, substitution or replacement of chemical groups and moieties on the amino acid or by derivatization of the amino acid. Amino acid mimetics include, for example, organic structures which exhibit functionally similar properties such as charge and charge spacing characteristic of the reference amino acid. For example, an organic structure which mimics Arginine (Arg or R) would have a positive charge moiety located in similar molecular space and having the same degree of mobility as the e-amino group of the side chain of the naturally occurring Arg amino acid. Mimetics also include constrained structures so as to maintain optimal spacing and charge interactions of the amino acid or of the amino acid functional groups. Those skilled in the art know or can determine what structures constitute functionally equivalent amino acid analogs and amino acid mimetics.

“Biocompatible” refers to materials or compounds which are generally not injurious to biological functions and which will not result in any degree of unacceptable toxicity, including allergenic and disease states.

By “coding sequence” is meant any nucleic acid sequence that contributes to the code for the polypeptide product of a gene. By contrast, the term “non-coding sequence” refers to any nucleic acid sequence that does not directly contribute to the code for the polypeptide product of a gene.

Throughout this disclosure, unless the context requires otherwise, the words “comprise,” “comprises,” and “comprising” will be understood to imply the inclusion of a stated step or element or group of steps or elements but not the exclusion of any other step or element or group of steps or elements.

By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The term “endotoxin free” or “substantially endotoxin free” relates generally to compositions, solvents, and/or vessels that contain at most trace amounts (e.g., amounts having no clinically adverse physiological effects to a subject) of endotoxin, and preferably undetectable amounts of endotoxin. Endotoxins are toxins associated with certain micro-organisms, such as bacteria, typically gram-negative bacteria, although endotoxins may be found in gram-positive bacteria, such as Listeria monocytogenes. The most prevalent endotoxins are lipopolysaccharides (LPS) or lipo-oligo-saccharides (LOS) found in the outer membrane of various Gram-negative bacteria, and which represent a central pathogenic feature in the ability of these bacteria to cause disease. Small amounts of endotoxin in humans may produce fever, a lowering of the blood pressure, and activation of inflammation and coagulation, among other adverse physiological effects.

Therefore, in pharmaceutical production, it is often desirable to remove most or all traces of endotoxin from drug products and/or drug containers, because even small amounts may cause adverse effects in humans. A depyrogenation oven may be used for this purpose, as temperatures in excess of 300° C. are typically required to break down most endotoxins. For instance, based on primary packaging material such as syringes or vials, the combination of a glass temperature of 250° C. and a holding time of 30 minutes is often sufficient to achieve a 3 log reduction in endotoxin levels. Other methods of removing endotoxins are contemplated, including, for example, chromatography and filtration methods, as described herein and known in the art.

Endotoxins can be detected using routine techniques known in the art. For example, the Limulus Amoebocyte Lysate assay, which utilizes blood from the horseshoe crab, is a very sensitive assay for detecting presence of endotoxin. In this test, very low levels of LPS can cause detectable coagulation of the limulus lysate due a powerful enzymatic cascade that amplifies this reaction. Endotoxins can also be quantitated by enzyme-linked immunosorbent assay (ELISA). To be substantially endotoxin free, endotoxin levels may be less than about 0.001, 0.005, 0.01, 0.02, 0.03, 0.04, 0.05, 0.06, 0.08, 0.09, 0.1, 0.5, 1.0, 1.5, 2, 2.5, 3, 4, 5, 6, 7, 8, 9, or 10 EU/mg of active compound. Typically, 1 ng lipopolysaccharide (LPS) corresponds to about 1-10 EU.

The “half-life” of a polypeptide can refer to the time it takes for the polypeptide to lose half of its pharmacologic, physiologic, or other activity, relative to such activity at the time of administration into the serum or tissue of an organism, or relative to any other defined time-point. “Half-life” can also refer to the time it takes for the amount or concentration of a polypeptide to be reduced by half of a starting amount administered into the serum or tissue of an organism, relative to such amount or concentration at the time of administration into the serum or tissue of an organism, or relative to any other defined time-point. The half-life can be measured in serum and/or any one or more selected tissues.

The terms “modulating” and “altering” include “increasing,” “enhancing” or “stimulating,” as well as “decreasing” or “reducing,” typically in a statistically significant or a physiologically significant amount or degree relative to a control. An “increased,” “stimulated” or “enhanced” amount is typically a “statistically significant” amount, and may include an increase that is 1.1, 1.2, 2, 3, 4, 5, 6, 7, 8, 9, 10, 15, 20, 30 or more times (e.g., 500, 1000 times) (including all integers and ranges in between e.g., 1.5, 1.6, 1.7. 1.8, etc.) the amount produced by no composition (e.g., the absence of agent) or a control composition. A “decreased” or “reduced” amount is typically a “statistically significant” amount, and may include a 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% decrease (including all integers and ranges in between) in the amount produced by no composition (e.g., the absence of an agent) or a control composition. Examples of comparisons and “statistically significant” amounts are described herein.

The terms “polypeptide,” “protein” and “peptide” are used interchangeably and mean a polymer of amino acids not limited to any particular length. The term “enzyme” includes polypeptide or protein catalysts, and with respect to ADI is used interchangeably with protein, polypeptide, or peptide. The terms include modifications such as myristoylation, sulfation, glycosylation, phosphorylation and addition or deletion of signal sequences. The terms “polypeptide” or “protein” means one or more chains of amino acids, wherein each chain comprises amino acids covalently linked by peptide bonds, and wherein said polypeptide or protein can comprise a plurality of chains non-covalently and/or covalently linked together by peptide bonds, having the sequence of native proteins, that is, proteins produced by naturally-occurring and specifically non-recombinant cells, or genetically-engineered or recombinant cells, and comprise molecules having the amino acid sequence of the native protein, or molecules having deletions from, additions to, and/or substitutions of one or more amino acids of the native sequence. The terms “polypeptide” and “protein” specifically encompass the ADI enzymes/proteins described herein, or sequences that have deletions from, additions to, and/or substitutions of one or more amino acid of the ADI proteins. In certain embodiments, the polypeptide is a “recombinant” polypeptide, produced by recombinant cell that comprises one or more recombinant DNA molecules, which are typically made of heterologous polynucleotide sequences or combinations of polynucleotide sequences that would not otherwise be found in the cell.

The term “isolated” polypeptide or protein referred to herein means that a subject protein (1) is free of at least some other proteins with which it would typically be found in nature, (2) is essentially free of other proteins from the same source, e.g., from the same species, (3) is expressed by a cell from a different species, (4) has been separated from at least about 50 percent of polynucleotides, lipids, carbohydrates, or other materials with which it is associated in nature, (5) is not associated (by covalent or non-covalent interaction) with portions of a protein with which the “isolated protein” is associated in nature, (6) is operably associated (by covalent or non-covalent interaction) with a polypeptide with which it is not associated in nature, or (7) does not occur in nature. Such an isolated protein can be encoded by genomic DNA, cDNA, mRNA or other RNA, of may be of synthetic origin, or any combination thereof. In certain embodiments, the isolated protein is substantially free from proteins or polypeptides or other contaminants that are found in its natural environment that would interfere with its use (therapeutic, diagnostic, prophylactic, research or otherwise).

In certain embodiments, the “purity” of any given agent (e.g., ADI-PEG) in a composition may be specifically defined. For instance, certain compositions may comprise an agent that is at least 70, 75 80, 85, 90, 91, 92, 93, 94, 95, 96, 97, 98, 99, or 100% pure (for example, on a protein basis), including all decimals and ranges in between, as measured, for example, by high performance liquid chromatography (HPLC), a well-known form of column chromatography used frequently in biochemistry and analytical chemistry to separate, identify, and quantify compounds.

The term “reference sequence” refers generally to a nucleic acid coding sequence, or amino acid sequence, to which another sequence is being compared. All polypeptide and polynucleotide sequences described herein are included as references sequences, including those described by name and those described in the Tables and the Sequence Listing.

The terms “sequence identity” or, for example, comprising a “sequence 50% identical to,” as used herein, refer to the extent that sequences are identical on a nucleotide-by-nucleotide basis or an amino acid-by-amino acid basis over a window of comparison. Thus, a “percentage of sequence identity” may be calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e., resulting in the highest percentage homology over the comparison window) generated by any of the various methods selected. Reference also may be made to the BLAST family of programs as for example disclosed by Altschul et al., Nucl. Acids Res. 25:3389, 1997.

The term “solubility” refers to the property of an agent (e.g., ADI-PEG) provided herein to dissolve in a liquid solvent and form a homogeneous solution. Solubility is typically expressed as a concentration, either by mass of solute per unit volume of solvent (g of solute per kg of solvent, g per dL (100 mL), mg/ml, etc.), molarity, molality, mole fraction or other similar descriptions of concentration. The maximum equilibrium amount of solute that can dissolve per amount of solvent is the solubility of that solute in that solvent under the specified conditions, including temperature, pressure, pH, and the nature of the solvent. In certain embodiments, solubility is measured at physiological pH, or other pH, for example, at pH 5.0, pH 6.0, pH 7.0, pH 7.4, pH 7.6, pH 7.8, or pH 8.0 (e.g., about pH 5-8). In certain embodiments, solubility is measured in water or a physiological buffer such as PBS or NaCl (with or without NaP). In specific embodiments, solubility is measured at relatively lower pH (e.g., pH 6.0) and relatively higher salt (e.g., 500 mM NaCl and 10 mM NaP). In certain embodiments, solubility is measured in a biological fluid (solvent) such as blood or serum. In certain embodiments, the temperature can be about room temperature (e.g., about 20, 21, 22, 23, 24, 25° C.) or about body temperature (37° C.). In certain embodiments, an agent has a solubility of at least about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 40, 50, 60, 70, 80, 90 or 100 mg/ml at room temperature or at 37° C.

A “subject” or a “subject in need thereof” or a “patient” or a “patient in need thereof” includes a mammalian subject such as a human subject.

“Substantially” or “essentially” means nearly totally or completely, for instance, 95%, 96%, 97%, 98%, 99% or greater of some given quantity.

By “statistically significant,” it is meant that the result was unlikely to have occurred by chance. Statistical significance can be determined by any method known in the art. Commonly used measures of significance include the p-value, which is the frequency or probability with which the observed event would occur, if the null hypothesis were true. If the obtained p-value is smaller than the significance level, then the null hypothesis is rejected. In simple cases, the significance level is defined at a p-value of 0.05 or less.

“Therapeutic response” refers to improvement of symptoms (whether or not sustained) based on administration of one or more therapeutic agents.

As used herein, “treatment” of a subject (e.g. a mammal, such as a human) or a cell is any type of intervention used in an attempt to alter the natural course of the individual or cell. Treatment includes, but is not limited to, administration of a pharmaceutical composition, and may be performed either prophylactically or subsequent to the initiation of a pathologic event or contact with an etiologic agent. Also included are “prophylactic” treatments, which can be directed to reducing the rate of progression of the disease or condition being treated, delaying the onset of that disease or condition, or reducing the severity of its onset. “Treatment” or “prophylaxis” does not necessarily indicate complete eradication, cure, or prevention of the disease or condition, or associated symptoms thereof.

The term “wild-type” refers to a gene or gene product (e.g., a polypeptide) that is most frequently observed in a population and is thus arbitrarily designed the “normal” or “wild-type” form of the gene.

Each embodiment in this specification is to be applied mutatis mutandis to every other embodiment unless expressly stated otherwise.

Throughout the present disclosure, the following abbreviations may be used: PEG, polyethylene glycol; ADI, arginine deiminase; SS, succinimidyl succinate; SSA, succinimidyl succinimide; SPA, succinimidyl propionate; NHS, N-hydroxy-succinimide; ASS-1, argininosuccinate synthetase-1.

### Lyophilized Formulations

Certain embodiments relate to lyophilized formulations, comprising a pegylated arginine deiminase (ADI-PEG), wherein the lyophilized formulation is sterile, substantially endotoxin-free, and at a pharmaceutically-acceptable pH. In some embodiments, the formulation comprises a pharmaceutically-acceptable buffer. In certain embodiments, the formulation comprises one or more pharmaceutically-acceptable excipients, including, for example, one or more cryoprotectants, lyoprotectants, stabilizers, bulking agents, tonicity modifiers, surfactants, pharmaceutical plasticizers, or chelators, including any combination of the foregoing.

Some formulations comprise one or more pharmaceutically-acceptable buffers. In certain embodiments, the buffer is at a concentration of about 0.10 mM to about 200 mM, or about 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1.0, 1.5, 2.0, 2.5, 3.0, 3.5, 4.0, 4.5, 5.0, 5.5, 6.0, 6.5. 7.0, 7.5, 8.0, 8.5, 9.0, 9.5, 10, 11, 12, 13, 14, 15, 16, 17, 18, 19, 20, 25, 30, 35, 40, 45, 50, 55, 60, 65, 70, 75, 80, 85, 90, 95, 100, 105, 110, 120, 125, 130, 135, 140, 145, 150, 155, 160, 165, 170, 175, 180, 185, 190, 195, or 200 mM, including all integers and ranges in between. In particular embodiments, the buffer is at a concentration of about 1 mM to about 50 mM, or about 10 mM to about 30 mM, or about 15 mM to about 25 mM, or about 10 mM to about 20 mM, or about 20 mM, or about 10 mM. In particular embodiments, the buffer is selected from one or more of histidine, sodium citrate, glycyl-glycine, sodium phosphate, Tris, lysine, and any combination of the foregoing.

In specific embodiments, the formulation comprises a histidine buffer at about 1 to about 50 mM, or about 10 to about 30 mM, or about 15 to about 25 mM, or about 20 mM. In some embodiments, the formulation comprises a sodium citrate at about 1 to about 50 mM, or about 10 to about 30 mM, or about 15 to about 25 mM, or about 20 mM. In particular embodiments, the formulation comprises a glycyl-glycine buffer at about 1 to about 50 mM, or about 10 to about 30 mM, or about 15 to about 25 mM, or about 20 mM. In specific embodiments, the formulation comprises a sodium phosphate buffer at about 1 to about 50 mM, or about 10 to about 30 mM, or about 15 to about 25 mM, or about 20 mM. In some embodiments, the formulation comprises a Tris buffer at about 1 to about 50 mM, or about 10 to about 30 mM, or about 15 to about 25 mM, or about 20 mM. In certain embodiments, the formulation comprises a lysine buffer at about 1 to about 50 mM, or about 10 to about 30 mM, or about 15 to about 25 mM, or about 20 mM.

Some formulations comprise one or more cryoprotectants. A “cryoprotectant” refers to a pharmaceutically-acceptable substance or excipient that protects the active agent(s) during the freezing stage(s) of lyophilization. In some embodiments, the cryoprotectant is present at about 0.001% to about 20% (wt %), or about 0.001, 0.002, 0.003, 0.004, 0.005, 0.006, 0.007, 0.008, 0.009, 0.010, 0.015, 0.020, 0.025, 0.030, 0.035, 0.040, 0.045, 0.050, 0.055, 0.060, 0.065, 0.070, 0.075, 0.080, 0.085, 0.090, 0.095, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1.0, 1.1, 1.2, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 5.0, 5.1, 5.2, 5.3, 5.4, 5.5, 5.6, 5.7, 5.8, 5.9, 6.0, 6.1. 6.2. 6.3. 6.4. 6.5. 6.6. 6.7. 6.8. 6.9. 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10, 11, 12, 13, 14, 15, 16, 17, 18, 19, or 20%, including all integers and ranges in between. In specific embodiments, the cryoprotectant is selected from one or more of sucrose, trehalose, ethylene glycol, propylene glycol, glycerol, and any combination of the foregoing.

Certain formulations comprise one or more lyoprotectants. A “lyoprotectant” refers to a pharmaceutically-acceptable substance or excipient that protects the active agent(s) during the drying stage(s) of lyophilization. In some embodiments, the lyoprotectant is present at about 0.001% to about 20% (wt %), or about 0.001, 0.002, 0.003, 0.004, 0.005, 0.006, 0.007, 0.008, 0.009, 0.010, 0.015, 0.020, 0.025, 0.030, 0.035, 0.040, 0.045, 0.050, 0.055, 0.060, 0.065, 0.070, 0.075, 0.080, 0.085, 0.090, 0.095, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1.0, 1.1, 1.2, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 5.0, 5.1, 5.2, 5.3, 5.4, 5.5, 5.6, 5.7, 5.8, 5.9, 6.0, 6.1. 6.2. 6.3. 6.4. 6.5. 6.6. 6.7. 6.8. 6.9. 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10, 11, 12, 13, 14, 15, 16, 17, 18, 19, or 20%, including all integers and ranges in between. In specific embodiments, the lyoprotectant is selected from one or more of sucrose, trehalose, mannitol, sorbitol, glycerol, and any combination of the foregoing.

Certain formulations comprise one or more pharmaceutically-acceptable stabilizers. In some embodiments, the stabilizer is present at about 0.001% to about 20% (wt %), or about 0.001, 0.002, 0.003, 0.004, 0.005, 0.006, 0.007, 0.008, 0.009, 0.010, 0.015, 0.020, 0.025, 0.030, 0.035, 0.040, 0.045, 0.050, 0.055, 0.060, 0.065, 0.070, 0.075, 0.080, 0.085, 0.090, 0.095, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1.0, 1.1, 1.2, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 5.0, 5.1, 5.2, 5.3, 5.4, 5.5, 5.6, 5.7, 5.8, 5.9, 6.0, 6.1. 6.2. 6.3. 6.4. 6.5. 6.6. 6.7. 6.8. 6.9. 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10, 11, 12, 13, 14, 15, 16, 17, 18, 19, or 20%, including all integers and ranges in between. In some embodiments, the stabilizer is selected from one or more of sucrose, mannitol, lactose, trehalose, maltose, sorbitol, gelatin, albumin, and any combination of the foregoing.

Some formulations comprise one or more pharmaceutically-acceptable bulking agents. In some embodiments, the bulking agent is present at about 0.001% to about 20% (wt %), or about 0.001, 0.002, 0.003, 0.004, 0.005, 0.006, 0.007, 0.008, 0.009, 0.010, 0.015, 0.020, 0.025, 0.030, 0.035, 0.040, 0.045, 0.050, 0.055, 0.060, 0.065, 0.070, 0.075, 0.080, 0.085, 0.090, 0.095, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1.0, 1.1, 1.2, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 5.0, 5.1, 5.2, 5.3, 5.4, 5.5, 5.6, 5.7, 5.8, 5.9, 6.0, 6.1. 6.2. 6.3. 6.4. 6.5. 6.6. 6.7. 6.8. 6.9. 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10, 11, 12, 13, 14, 15, 16, 17, 18, 19, or 20%, including all integers and ranges in between. In specific embodiments, the bulking agent is selected from one or more of mannitol, sorbitol, lactose, glucose, sucrose, glycine, albumin, dextran 40, and any combination of the foregoing.

Some formulations comprise one or more pharmaceutically-acceptable tonicity modifiers. In some embodiments, the tonicity modifier is present at about 0.001% to about 20% (wt %), or about 0.001, 0.002, 0.003, 0.004, 0.005, 0.006, 0.007, 0.008, 0.009, 0.010, 0.015, 0.020, 0.025, 0.030, 0.035, 0.040, 0.045, 0.050, 0.055, 0.060, 0.065, 0.070, 0.075, 0.080, 0.085, 0.090, 0.095, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1.0, 1.1, 1.2, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 5.0, 5.1, 5.2, 5.3, 5.4, 5.5, 5.6, 5.7, 5.8, 5.9, 6.0, 6.1. 6.2. 6.3. 6.4. 6.5. 6.6. 6.7. 6.8. 6.9. 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10, 11, 12, 13, 14, 15, 16, 17, 18, 19, or 20%, or at a concentration of about 0.10 mM to about 200 mM, or about 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1.0, 1.5, 2.0, 2.5, 3.0, 3.5, 4.0, 4.5, 5.0, 5.5, 6.0, 6.5. 7.0, 7.5, 8.0, 8.5, 9.0, 9.5, 10, 11, 12, 13, 14, 15, 16, 17, 18, 19, 20, 25, 30, 35, 40, 45, 50, 55, 60, 65, 70, 75, 80, 85, 90, 95, 100, 105, 110, 120, 125, 130, 135, 140, 145, 150, 155, 160, 165, 170, 175, 180, 185, 190, 195, or 200 mM, including all integers and ranges in between. In particular embodiments, the tonicity modifier is selected from one or more of sodium chloride, sucrose, mannitol, and any combination of the foregoing.

In certain embodiments, the formulation comprises one or more surfactants. In some embodiments, surfactant is present at about 0.001% to about 20% (wt %), or about 0.001, 0.002, 0.003, 0.004, 0.005, 0.006, 0.007, 0.008, 0.009, 0.010, 0.015, 0.020, 0.025, 0.030, 0.035, 0.040, 0.045, 0.050, 0.055, 0.060, 0.065, 0.070, 0.075, 0.080, 0.085, 0.090, 0.095, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1.0, 1.1, 1.2, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 5.0, 5.1, 5.2, 5.3, 5.4, 5.5, 5.6, 5.7, 5.8, 5.9, 6.0, 6.1. 6.2. 6.3. 6.4. 6.5. 6.6. 6.7. 6.8. 6.9. 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10, 11, 12, 13, 14, 15, 16, 17, 18, 19, or 20%. Exemplary surfactants include Tween-80, polysorbate 20 (P20), polysorbate 80 (P80), poloxamer 188, and combinations thereof.

Certain formulations comprise one or more pharmaceutical plasticizers. In some embodiments, the pharmaceutical plasticizer is present at about 0.001% to about 20% (wt %), or about 0.001, 0.002, 0.003, 0.004, 0.005, 0.006, 0.007, 0.008, 0.009, 0.010, 0.015, 0.020, 0.025, 0.030, 0.035, 0.040, 0.045, 0.050, 0.055, 0.060, 0.065, 0.070, 0.075, 0.080, 0.085, 0.090, 0.095, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1.0, 1.1, 1.2, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 5.0, 5.1, 5.2, 5.3, 5.4, 5.5, 5.6, 5.7, 5.8, 5.9, 6.0, 6.1. 6.2. 6.3. 6.4. 6.5. 6.6. 6.7. 6.8. 6.9. 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10, 11, 12, 13, 14, 15, 16, 17, 18, 19, or 20%, including all integers and ranges in between. In particular embodiments, the pharmaceutical plasticizer is glycerol.

Some formulations comprise one or more chelators. In some embodiments, the chelator is present at about 0.001% to about 1% (wt %), or about 0.001, 0.002, 0.003, 0.004, 0.005, 0.006, 0.007, 0.008, 0.009, 0.010, 0.015, 0.020, 0.025, 0.030, 0.035, 0.040, 0.045, 0.050, 0.055, 0.060, 0.065, 0.070, 0.075, 0.080, 0.085, 0.090, 0.095, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, or 1.0%, including all integers and ranges in between. In specific embodiments, the chelator is ethylenediaminetetraacetic acid (EDTA).

Particular formulations comprise a pharmaceutically-acceptable excipient selected from one or more of sucrose, trehalose, dextran, mannitol, proline, glycine, a surfactant, a pharmaceutical plasticizer, a chelator, and any combination of the foregoing. For example, in some embodiments the sucrose, trehalose, dextran (for example dextran 40), mannitol, proline, and/or glycine is present at about 0.001% to about 20% (wt %), or about 0.001, 0.002, 0.003, 0.004, 0.005, 0.006, 0.007, 0.008, 0.009, 0.010, 0.015, 0.020, 0.025, 0.030, 0.035, 0.040, 0.045, 0.050, 0.055, 0.060, 0.065, 0.070, 0.075, 0.080, 0.085, 0.090, 0.095, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1.0, 1.1, 1.2, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 5.0, 5.1, 5.2, 5.3, 5.4, 5.5, 5.6, 5.7, 5.8, 5.9, 6.0, 6.1. 6.2. 6.3. 6.4. 6.5. 6.6. 6.7. 6.8. 6.9. 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10, 11, 12, 13, 14, 15, 16, 17, 18, 19, or 20%, or at a concentration of about 0.10 mM to about 200 mM, or about 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1.0, 1.5, 2.0, 2.5, 3.0, 3.5, 4.0, 4.5, 5.0, 5.5, 6.0, 6.5. 7.0, 7.5, 8.0, 8.5, 9.0, 9.5, 10, 11, 12, 13, 14, 15, 16, 17, 18, 19, 20, 25, 30, 35, 40, 45, 50, 55, 60, 65, 70, 75, 80, 85, 90, 95, 100, 105, 110, 120, 125, 130, 135, 140, 145, 150, 155, 160, 165, 170, 175, 180, 185, 190, 195, or 200 mM, including all integers and ranges in between.

In specific embodiments, as noted above, the formulation comprises a histidine buffer at about 1 to about 50 mM, or about 10 to about 30 mM, or about 15 to about 25 mM, or about 20 mM.

In some embodiments, the formulation comprises a histidine buffer at about 1 to about 50 mM, or about 10 to about 30 mM, or about 15 to about 25 mM, or about 20 mM, and comprises sucrose at about 1% to about 10%, or about 4% to about 6%, or about 5%.

In some embodiments, the formulation comprises a histidine buffer at about 1 to about 50 mM, or about 10 to about 30 mM, or about 15 to about 25 mM, or about 20 mM, and comprises mannitol at about 1% to about 10%, or about 4% to about 6%, or about 5%.

In some embodiments, the formulation comprises a histidine buffer at about 1 to about 50 mM, or about 10 to about 30 mM, or about 15 to about 25 mM, or about 20 mM, and comprises dextran, for example, dextran 40, at about 0.1% to about 5%, or about 0.5% to about 2%, or about 1%.

In particular embodiments, the formulation comprises a histidine buffer at about 1 to about 50 mM, or about 10 to about 30 mM, or about 15 to about 25 mM, or about 20 mM, and comprises dextran, example, dextran 40, at about 0.1% to about 5%, or about 0.5% to about 2%, or about 1%, and comprises sucrose at about 1% to about 10%, or about 4% to about 6%, or about 5%.

In certain embodiments, the formulation comprises a histidine buffer at about 1 to about 50 mM, or about 10 to about 30 mM, or about 15 to about 25 mM, or about 20 mM, and comprises trehalose at about 1% to about 10%, or about 4% to about 6%, or about 5%.

In some embodiments, the formulation comprises a histidine buffer at about 1 to about 50 mM, or about 10 to about 30 mM, or about 15 to about 25 mM, or about 20 mM, and comprises mannitol at about 1% to about 10%, or about 3% to about 5%, or about 4%, and comprising sucrose at about 0.1% to about 5%, or about 0.5% to about 2%, or about 1%.

In some embodiments, the formulation comprises a histidine buffer at about 1 to about 50 mM, or about 10 to about 30 mM, or about 15 to about 25 mM, or about 20 mM, and comprises sucrose at about 1% to about 10%, or about 4% to about 6%, or about 4.8% or 5%, and comprising Tween-80 at about 0.001% to about 0.1%, or about 0.005% to about 0.05%, or about 0.01%.

In some embodiments, the formulation comprises a histidine buffer at about 1 to about 50 mM, or about 10 to about 30 mM, or about 15 to about 25 mM, or about 20 mM, and comprises sucrose at about 1% to about 10%, or about 4% to about 6%, or about 4.8% or 5%, comprising Tween-80 at about 0.001% to about 0.1%, or about 0.005% to about 0.05%, or about 0.01%, and comprising glycerol at about 0.01% to about 1.0%, or about 0.1% to about 0.5%, or about 0.25%.

In some embodiments, the formulation comprises a histidine buffer at about 1 to about 50 mM, or about 10 to about 30 mM, or about 15 to about 25 mM, or about 20 mM, and comprises trehalose at about 1% to about 15%, or about 5% to about 12%, or about 10% or 9.5%.

In some embodiments, the formulation comprises a histidine buffer at about 1 to about 50 mM, or about 10 to about 30 mM, or about 15 to about 25 mM, or about 20 mM, and comprises trehalose at about 1% to about 15%, or about 5% to about 12%, or about 10% or 9.5%, and comprises proline at about 10 mM to about 40 mM, or about 15 to about 30 mM, or about 20 mM.

In some embodiments, the formulation comprises a histidine buffer at about 1 to about 50 mM, or about 10 to about 30 mM, or about 15 to about 25 mM, or about 20 mM, and comprises trehalose at about 5% to about 15%, or about 8% to about 12%, or about 10% or 9.5%, and comprises glycine at about 10 mM to about 40 mM, or about 15 to about 30 mM, or about 20 mM.

In some embodiments, the formulation comprises a histidine buffer at about 1 to about 50 mM, or about 10 to about 30 mM, or about 15 to about 25 mM, or about 20 mM, and comprises sucrose at about 1% to about 15%, or about 5% to about 12%, or about 10% or 9.5%.

In some embodiments, the formulation comprises a histidine buffer at about 1 to about 50 mM, or about 10 to about 30 mM, or about 15 to about 25 mM, or about 20 mM, and comprises sucrose at about 1% to about 10%, about 4% to about 6%, or about 5% or 4.5%, and comprises trehalose at about 1% to about 10%, about 4% to about 6%, or about 5% or 4.5%.

In particular embodiments, the formulation comprises a histidine buffer at about 1 to about 50 mM, or about 10 to about 30 mM, or about 15 to about 25 mM, or about 20 mM, and comprises trehalose at about 5% to about 15%, or about 8% to about 12%, or about 10% or 9.5%, and comprises EDTA at about 0.01% to about 0.1%, or about 0.02% to about 0.08%, or about 0.05%.

Certain formulations comprise a histidine buffer at about 1 to about 30 mM, or about 5 to about 20 mM, or about 10 mM, and comprise glycine at about 1% to about 5%, or about 2% or 1.9%, comprise trehalose at about 0.1% to about 2%, or about 0.5% to about 1.5%, or about 1%, and comprise a surfactant, for example, P20, at about 0.001% to about 0.1%, or about 0.005% to about 0.02%, or about 0.01%. Some formulations comprise a histidine buffer at about 1 to about 30 mM, or about 5 to about 20 mM, or about 10 mM, and comprise sucrose at about 1% to about 15%, or about 5% to about 12%, or about 10% or 8.5%, and comprise trehalose at about 0.1% to about 2%, or about 0.5% to about 1.5%, or about 1%. Particular formulations comprise a histidine buffer at about 1 to about 30 mM, or about 5 to about 20 mM, or about 10 mM, and comprise sucrose at about 1% to about 15%, or about 5% to about 12%, or about 10% or 9%. Some formulations comprise a histidine buffer at about 1 to about 50 mM, or about 10 to about 40 mM, or about 20, 25, 30, or 35 mM, and comprise a sodium chloride at about 100 to about 150 mM, or about 120, 130, or 140 mM. Certain formulations comprise a sodium phosphate buffer at about 100 to about 200 mM, or about 150 mM, and comprise sucrose and/or trehalose at about 1% to about 10%, about 4% to about 6%, or about 5% or 4.5%, and comprise glycine at about 0.1 to about 1%, or about 0.5%, and comprise glycerol at about 0.1 to about 1.0%, or about 0.25%, and comprise Tween 80 at about 0.001 to about 0.1%, or about 0.01%, and comprise EDTA at about 0.01 to about 0.1%, or about 0.05%.

In some embodiments, the pharmaceutically-acceptable pH of the formulation is about 5.0 to about 8.0 (±0.01 to 0.1 or to 1.0), or about 5.0, 5.1, 5.2, 5.3, 5.4, 5.5, 5.6, 5.7, 5.8, 5.9, 6.0, 6.1. 6.2. 6.3. 6.4. 6.5. 6.6. 6.7. 6.8. 6.9. 7.0, 7.1, 7.2, 7.3, 7.4, 7.5, 7.6, 7.7, 7.8, 7.9, or 8.0 (±0.01 to +0.1), including all integers and ranges in between. In specific embodiments, the formulation comprising ADI-PEG has a pH of about 6.8 (±1.0). In particular embodiments, the pH is about 6.5 to about 7.2 (±0.1), or about 6.6 to about 7.0 (±0.1), or about 6.8 (±0.1). In some embodiments, the pH is about 6.0 to about 7.2 (±0.1), or at about 6.4 to about 6.8 (±0.1), or about 6.5 (±0.1).

As noted above, the formulations described herein comprise one or more pegylated arginine deiminase (ADI-PEG) molecules, or arginine deiminase (ADI) polypeptides that are modified by covalent attachment to one or more polyethylene glycol (PEG) molecules. When compared to unmodified ADI, ADI-PEG retains most of its enzymatic activity, is less immunogenic or antigenic, has a greatly extended circulating half-life, and is more efficacious in the treatment of tumors.

In certain embodiments, the dry weight of the ADI-PEG in the lyophilized formulation is about 50 mg/g to about 150 mg/g, or about 50, 55, 60, 65, 70, 75, 80, 85, 90, 95, 100, 105, 110, 120, 125, 130, 135, 140, 145, or 150 mg/g, including all integers and ranges in between.

In certain embodiments, an ADI polypeptide or ADI-PEG molecule has an “ADI activity”, or the ability to convert or metabolize arginine into citrulline and ammonia. ADI activity can be measured according to routine techniques in the art. For instance, the amount of L-citrulline can be detected by a colorimetric endpoint assay (see, for example, Knipp and Vasak, Analytical Biochem. 286:257-264, 2000) and compared to a standard curve of known amounts of L-citrulline in order to calculate the specific activity of ADI, which can be expressed as IU/mg of protein. In some embodiments, one IU of ADI enzyme activity is defined as the amount of enzyme that produces 1 gmol of citrulline per minute at the pH and temperature being tested.

The ADI portion of the ADI-PEG molecule(s) can be derived from a variety of sources. For example, in some embodiments, the ADI polypeptide is from M. hominis, M. arginini, M. arthritidis, M. phocicerebrale, M. gateae, M. phocidae, M. columbinum, M. iowae, M. crocodyli, M. alligatoris, H. orenii, or M. bovis. In some embodiments, the ADI polypeptide is from Mycoplasma salivarium, Mycoplasma spumans, Mycoplasma canadense, Mycoplasma auris, Mycoplasma hyosynoviae, Mycoplasma cloacale, Mycoplasma anseris, Mycoplasma alkalescens, Mycoplasma orale, Mycoplasma iners, Mycoplasma meleagridis, Mycoplasma alvi, Mycoplasma penetrans, Mycoplasma gallinarum, Mycoplasma pirum, Mycoplasma primatum, Mycoplasma fermentans, Mycoplasma lipofaciens, Mycoplasma felifaucium, Mycoplasma imitans, Mycoplasma opalescens, Mycoplasma moatsii, Mycoplasma elephantis, Mycoplasma pneumoniae, Mycoplasma testudinis, Mycoplasma sp. CAG:877, or Mycoplasma sp. CAG:472.

Illustrative ADI polypeptides are provided in Table A1 below.

TABLE A1
ADI Polypeptide Sequences

| | | SEQ |
|---|---|---|
| | | ID |
| Source | Sequence | NO: |
| Mycoplasma | MSVFDSKFNGIHVYSEIGELETVLVHEPGREIDYITPARLDELLFSAILESHD | 1 |
| hominis | ARKEHQSFVKIMKDRGINVVELTDLVAETYDLASKAAKEEFIETFLEETVPVL | |
| | TEANKKAVRAFLLSKPTHEMVEFMMSGITKYELGVESENELIVDPMPNLYFTR | |
| | DPFASVGNGVTIHFMRYIVRRRETLFARFVFRNHPKLVKTPWYYDPAMKMPIE | |
| | GGDVFIYNNETLVVGVSERTDLDTITLLAKNIKANKEVEFKRIVAINVPKWTN | |
| | LMHLDTWLTMLDKNKFLYSPIANDVFKFWDYDLVNGGAEPQPQLNGLPLDKLL | |
| | ASIINKEPVLIPIGGAGATEMEIARETNFDGTNYLAIKPGLVIGYDRNEKTNA | |
| | ALKAAGITVLPFHGNQLSLGMGNARCMSMPLSRKDVKW | |
| PHX8 | MSVFDSKFNGIHVYSEIGELETVLVHEPGREIDYITPARLDELLFSAILESHD | 2 |
| | ARKEHQSFVKIMKDRGINVVELTDLVAETYDLASKAAKEEFIETFLEETVPVL | |
| | TEANKEAVRAFLLSKPTHEMVEFMMSGITKYELGVESENELIVDPMPNLYFTR | |
| | DPFASVGNGVTIHFMRYIVRRRETLFARFVFRNHPKLVKTPWYYDPAMKMSIE | |
| | GGDVFIYNNETLVVGVSERTDLDTITLLAKNIKANKEVEFKRIVAINVPKWTN | |
| | LMHLDTWLTMLDKNKFLYSPIANDVFKFWDYDLVNGGAEPQPQLNGLPLDKLL | |
| | ASIINKEPVLIPIGGAGATEMEIARETNFDGTNYLAIKPGLVIGYDRNEKTNA | |
| | ALKAAGITVLPFHGNQLSLGMGNARCMSMPLSRKDVKW | |
| Mycoplasma | IHVYSEIGELETVLVHEPGREIDYITPARLDELLFSAILESHDARKEHQSFVK | 3 |
| phocicerebrale | QLKDNGINVVELTDLVAETFDLASKEEQEKLIEEFLEDSEPVLSEAHKTAVRK | |
| | FLTSRKSTREMVEFMMAGITKYDLGIEADHELIVDPMPNLYFTRDPFASVGNG | |
| | VTIHYMRYKVRQRETLFSRFVFSNHPKLVKTPWYYDPAMKMSIEGGDVFIYNN | |
| | DTLVVGVSERTDLETITLLAKNIKANKEVEFKRIVAINVPKWTNLMHLDTWLT | |
| | MLDKDKFLYSPIANDVFKFWDYDLVNGGAEPQPKENGLPLEGLLQSIINKKPV | |
| | LIPIAGNNASHIDIERETHFDGTNYLAIKPGVVIGYARNEKTNAALAAAGIKV | |
| | LPFHGNQLSLGMGNARCMSMP | |
| Mycoplasma | MSVFDSKFKGIHVYSEIGELESVLVHEPGREIDYITPARLDELLFSAILESHD | 4 |
| arginini | ARKEHKQFVAELKANDINVVELIDLVAETYDLASQEAKDKLIEEFLEDSEPVL | |
| | SEEHKVVVRNFLKAKKTSRELVEIMMAGITKYDLGIEADHELIVDPMPNLYFT | |
| | RDPFASVGNGVTIHYMRYKVRQRETLFSRFVFSNHPKLINTPWYYDPSLKLSI | |
| | EGGDVFIYNNDTLVVGVSERTDLQTVTLLAKNIVANKECEFKRIVAINVPKWT | |
| | NLMHLDTWLTMLDKDKFLYSPIANDVFKFWDYDLVNGGAEPQPVENGLPLEGL | |
| | LQSIINKKPVLIPIAGEGASQMEIERETHFDGTNYLAIRPGVVIGYSRNEKTN | |
| | AALEAAGIKVLPFHGNQLSLGMGNARCMSMPLSRKDVKW | |
| Mycoplasma | MSVFDSKFKGIHVYSEIGELETVLVHEPGKEIDYITPARLDELLFSAILESHD | 5 |
| arthritidis | ARKEHKEFVAELKKRGINVVELVDLIVETYDLASKEAKEKLLEEFLDDSVPVL | |
| | SDEHRAAVKKFLQSQKSTRSLVEYMIAGITKHDLKIESDLELIVDPMPNLYFT | |
| | RDPFASVGNGVTIHYMRYKVRQRETLFSRFVFSNHPKLVNTPWYYDPAEGLSI | |
| | EGGDVFIYNNDTLVVGVSERTDLQTITLLAKNIKANKECEFKRIVAINVPKWT | |
| | NLMHLDTWLTMLDKDKFLYSPIANDVFKFWDYDLVNGGDAPQPVDNGLPLEDL | |
| | LKSIIGKKPTLIPIAGAGASQIDIERETHFDGTNYLAVAPGIVIGYARNEKTN | |
| | AALEAAGITVLPFRGNQLSLGMGNARCMSMPLSRKDVK | |
| Mycoplasma | SVFSDKFNGIHVYSEIGDLESVLVHEPGKEIDYITPARLDELLFSAILESTDA | 6 |
| orale | RKEHKEFVEILKKQGINVVELVDLVVETYNLVDKKTQEKLLKDFLDDSEPVLS | |
| | PEHRKAVEKFLKSLKSTKELIQYMMAGITKYDLGIKADKELIVDPMPNLYFTR | |
| | DPFASVGNGVTIHYMRYKVRQRETLFSKFIFTNHPKLVKTPXYYDPAMKLSIE | |
| | GGDVFIYNNDTLVVGVSERTDLETITLLAKNIKANKECEFKRIVAINVPKXTN | |
| | LMHLDTXLTMLDKDKFLYSPIANDVFKFXDYDLVNGGSNPEPVVNGLPLDKLL | |
| | ESIINKKPVLIPIAGKGATEIETAVETHFDGTNYLAIKPGVVVGYSRNVKTNA | |
| | ALEANGIKVLPFKGNQLSLGMGNARCMSMPLSRKDVK | |
| Mycoplasma | IHVYSEIGELESVLVHEPGREIDYITPARLDELLFSAILESHDARKEHKLFVS | 7 |
| gateae | ELKANDINVVELTDLVTETYDLASQEAKDNLIEEFLEDSEPVLTEELKSVVRT | |
| | YLKSIKSTRELIQMMMAGITKYDLGIEADHELIVDPMPNLYFTRDPFASVGNG | |
| | VTIHYMRYKVRQRETLFSRFVFSNHPKLVNTPWYYDPSLKLSIEGGDVFIYNN | |
| | NTLVVGVSERTDLETVTLLAKNIVANKECEFKRIVAINVPKWTNLMHLDTWLT | |
| | MLDKDKFLYSPIANDVFKFWDYDLVNGGEEPQPVENGLPLEGLLESIINKKPI | |
| | LIPIAGEGASQIDIERETHFDGTNYLAIRPGVVIGYSRNEKTNAALEAAGIKV | |
| | LPFHGNQLSLGMGNARCMSM | |
| Mycoplasma | IHVYSEIGELQTVLVHEPGREIEYITPARLDELLFSAILESHDARKEHQEFVA | 8 |
| phocidae | ELKKNNINVVELTDLVSETYDMVSKEKQEKLIEEFLEDSEPVLSEEHKGLVRK | |
| | FLKSLKSSKELIQYMMAGITKHDLNIEADHELIVDPMPNLYFTRDPFASVGNG | |
| | VTIHYMRYKVRQRETLFSRFIFANHPKLMNTPLYYNPDMKLSIEGGDVFVYNN | |
| | ETLVVGVSERTDLDTITLLAKNIKANKEREFKRIVAINVPKWTNLMHLDTWLT | |
| | MLDKDKFLYSPIANDVFKFWDYDLVNGGDEPQPKVNGLPLEKLLESIINKKPI | |
| | LIPIAGTSASNIDVERETHFDGTNYLAIAPGVVIGYSRNVKTNEALEAAGIKV | |
| | LPFKGNQLSLGMGNARCMSMP | |
| Mycoplasma | MSKINVYSEIGELKEVLVHTPGDEIRRISPSRLDELLFSAILEPNEAIKEHKG | 9 |
| columbinum | FLKILQDKGIKVIQLSDLVAETYTYHATQKEREAFIEKWLDEAEPALTKDLRA | |
| | KVKSYVLSKEGTPVAMVRTMMAGVSKQELNVESETELVVDPMPNLYFTRDPFA | |
| | SAGNGISLNNMKYVTRKRETIFAEFIFATHPDYKTTPHWFDRLDEGNIEGGDV | |
| | FIYNKDTLVIGVSERTNKEAILTIAKKIKNNKEAKFKKIVAINVPPMPNLMHL | |
| | DTWLTMVDKDKFLYSPNMLSVLKVWEIDLSKEIEMVETNKPLADVLESIIGVK | |
| | PVLIPIAGKGATQLDIDIETHFDGTNYLTIAPGVVVGYSRNIKTEAALRAAGV | |
| | TVLSFEGNQLSLGMGSARCMSMPLVREDVK | |
| Mycoplasma | MGNNIPKKINVFSEIGNLKRVLVHTPGKEIEYVTPQRLDELLFSAILDPVRAR | 10 |
| iowae | EEHKEFIKILESQGVEVVQLVDLTAETYDVAESQAKENFIQKWLDESLPKLTD | |
| | ENRNKVYSLLKSLEKDPKEMIRKMMSGVLASEIGVKSDVELIVDPMPNLYFTR | |
| | DPFASVGNGITLHRMFRPTRRRETIFADFIFSNHPEYKSTQKYYEREDKFSLE | |
| | GGDVFIYNNKTLVVGVSERTEKGAIKALAKAVQNNSNMSFEKIYAINVPKMSN | |
| | LMHLDTWLTMLDTDKFLYSPNMMGVLKIWEIDLSDKSLKWKEIRDSLDHFLST | |
| | IIGKKAITVPVAGKDAMQFEIDIETHFDATNFIAVAPGVVIGYDRNKKTNEAL | |
| | KEAGIKVLSWNGDQLSLGMGSARCMTMPLYREELKK | |
| Mycoplasma | MNKINVYSEVGKLKEVLVHTPGDEIRRISPSRLEELLFSAILEPDSAIEEHKR | 11 |
| crocodyli | FLKILEDNNIKVIQLDQLVADTYELVNPSVRDAFIEKWLNESEPKLDKKLREK | |
| | VKEYLLHTQKTVGTKRMVRIMMAGVDRVELGVELDRQLVVDPMPNLYFTRDPF | |
| | ASAGNGISLNNMKYVTRKRETIFSEFIFENHPDYKTTPHWFDRLDKGNIEGGD | |
| | VFIYNRTTLVIGISERTNKDALLTIANNIKSNKESKFERIVAVNVPPMPNLMH | |
| | LDTWLTMVDHDKFLYSPNMMKTLKFWTIDLTKPIKMVELEESLSDMIETIIGK | |
| | KPVLIPIAGHDASPLDVDIETHFDGTNYLTIAPGVVVGYSRNKLTEKALTKAG | |
| | VKVLSFEGNQLSLGMGSARCMSMPLVREDIK | |
| Mycoplasma | MQIIAKIDLLTNMLIFMKIYFIGRLIMKKINVYSEYGKLKEVLVHTPGDEIRR | 12 |
| fermentans | LAPSRLDELLFSAILEPDSAIAEHKRFVQLLKDNGIKVIQLDELFAKTFDLVS | |
| | ESVKQSLIERWLDECEPKLDATLRAKVKEYILELKAKSSKKMVRVMMAGIDKK | |
| | ELGIELDRDLVVDPMPNLYFTRDPFASVGNGISLHHMKYVTRQRETIFSEFIF | |
| | DNNLDYNTVPRWFDRKDEGRIEGGDVFIYSADTLVVGVSERTNKEAINVMARK | |
| | IAADKEVKFKRIYAINVPPMPNLMHLDTWLTMLDKNKFLYSPNMLSVLKVWRI | |
| | DLNDPDFVWHEIEGSLEEILEQIIGMKPILIPIAGKGASQLDIDIETHFDGTN | |
| | YLTIAPSVVVGYSRNEKTEKALKAAKVKVLSFEGNQLSLGMGSARCMSMPLIR | |
| | EDIKKK | |
| Mycoplasma | MVITIALNILNKIYFKPQNRSILKLYRLPSLCTQISIFIGGKMSSIDKNSLGN | 13 |
| penetrans | GINVYSEIGELKEVLVHTPGDEIRYTAPSRLEELLFSAVLKADTAIEEHKGFV | |
| | KILQNNGIKVIQLCDLVAETYELCSKEVRNSFIEQYLDEALPVLKKEIRPVVK | |
| | DYLLSFPTVQMVRKMMSGILANELNIKQDNPLIIDGMPNLYFTRDPFASMGNG | |
| | VSINCMKYPTRKREVIFSRFVFTNNPKYKNTPRYFDIVGNNGTIEGGDIFIYN | |
| | SKTLVIGNSERTNFAAIESVAKNIQANKDCTFERIVVINVPPMPNLMHLDTWL | |
| | TMLDYDKFLYSPNMMNVLKIWEIDLNVKPVKFVEKKGTLEEVLYSIIDKKPIL | |
| | IPIAGKGANQLDIDIETHFDGTNYLTIAPGVVVGYERNEKTQKALVEAGIKVL | |
| | SFNGSQLSLGMGSARCMSMPLIRENLKK | |
| Mycoplasma | MFNKIRVYSEIGKLRKVLVHTPGKELDYVTPQRLDELLFSSLLNPIKARQEHE | 14 |
| gallisepticum | TFIKLLEDHDVECVQLSTLTAQTFQAMNSKIQEEFINRWLDECLPVLSEINRL | |
| | KVYDYLKSLATNPQVMIRKMMSGILAKEVGIQSEVELVADPMPNLYFTRDPFA | |
| | SIGKGITLHSMFHPTRKRETIFADFIFSHHPEYKNAPKYYSREDKYSIEGGDL | |
| | FVYDDKTLVIGVSERTEKKAIQSLAEKLRQNDETSFEKIYAINVPKMSNLMHL | |
| | DTWLTMLDYDKFLYSPNMMGVLKIWEIDLIHPTLIWRELNESLEGFLSMVIGK | |
| | KATLIPVAGEDSTQIEIDVETNFDATNFLVIQPGVVVGYDRNYKTNQALRDAG | |
| | VKVISWNGDQLSLGMGSARCMSMPLYRDPIKK | |
| Mycoplasma | MSKINVYSEVGRLKEVLVHTPGDEIRRISPTRLEELLFSAILEPDTAIEEHKR | 15 |
| alligatoris | FLNVLEKNGIKAIQLDELVAQTYDQVDQKIKDEFIDQWLQEAKPVLNDQLKKL | |
| | VKNYLLKSQKEFSTKKMVRIMMAGIDKKEINIDLDRDLVVDPMPNLYFTRDPF | |
| | ASVGNGISLHNMKYQTRKRETIFAQFIFKYNKDYKTTPHWFDRFDHGSIEGGD | |
| | VFVYTKDTLVIGISERTTKEAVLNIAKKIKANTDSKFKKIVAINVPPMPNLMH | |
| | LDTWITMVDHDKFLYSPNMMKSLKFWLIDLSKEIKMVELEESLSNMLEAIIGK | |
| | KPILIPIAGKNASQLDIDIETHFDGTNYLTIAPGVVVGYSRNKLTQKALEDAG | |
| | VKVLSFDGNQLSLGMGSARCMSMPLVREDIK | |
| Mycoplasma | MSKKQLVKTDGHNQLDQPNTKALQLKKKQFNSGVRVTSEISFLREVIAHHPGI | 16 |
| pneumoniae | ETERVIDNQTFGSAMYLERAQKEHQLFIKILRQHGTKVHYLQDLLLEALSAAD | |
| | PNVRQDFIKNFLLESGIKSVSTFEACLNFFRSLDSLVDVIKVMFGGIKVSDVP | |
| | PITPQRFADIHVSNSPFLIKPLSFSLYPHKFFNTLGTGVALFVTNDSELKRHS | |
| | LVYEYIMRFHPRFDGVKLYTNRDFKNCLINSSDIIQISNEILLIGISHDTDVL | |
| | GIESLARNLLSDHTNPIKQIIAINIHKFGAKTNLNKLIAMVDVDKFIIARKVL | |
| | QATEIFELTATAQRDVDGLAQIKFKPLKFNFGEIIEAIIDKQPRFVIIGGGDE | |
| | VAERKELLDCGMGVLNLSPGEIVVFDRNHYTNNLLNELGLIIHKIPASELSRG | |
| | PSGPLEMVCSLWRE | |
| Mycoplasma | MKDTKDIINVFSEIGELKKVLIHTPGNELKYVSPYRLDELLFSNVLEWREAKK | 17 |
| mobile | EHNEFIQKLKSEGVEPVELTDLVAESFEESSIKVKNDFIRQYLDEATPILDGL | |
| | TKQKLLPFFLDIKHSTRKTIELMMSGITQKDISISHIERELIIDPMPNLYFSR | |
| | DNFISIGNSVIISNMKYKTRKRETIFTDFIFKNHPLYKKVNMAFERKDLNNQI | |
| | SIIEGGDVLVYSKEILIIGISERTTMSAILELAENFKKTKRSFKKIYGVEVPK | |
| | MKNLMHLDTWLTMIDYDKFIYSPNVLTDLKFWEINLDYEKISSKELHASLSEF | |
| | LKLIIGKDPILIPIGGKGASQITIDIETNFVAANYLVIRPGVVIGYSRNYETQ | |
| | KALEGHGVKVIAFEGNQLSLGMGSSRCMSMPLIRSNLK | |
| Streptococcus | MTAQTPIHVYSEIGKLKKVLLHRPGKEIENLMPDYLERLLFDDIPFLEDAQKE | 18 |
| pyogenes | HDAFAQALRDEGIEVLYLETLAAESLVTPEIREAFIDEYLSEANIRGRATKKA | |
| | IRELLMAIEDNQELIEKTMAGVQKSELPEIPASEKGLTDLVESNYPFAIDPMP | |
| | NLYFTRDPFATIGTGVSLNHMFSETRNRETLYGKYIFTHHPIYGGGKVPMVYD | |
| | RNETTRIEGGDELVLSKDVLAVGISQRTDAASIEKLLVNIFKQNLGFKKVLAF | |
| | EFANNRKFMHLDTVFTMVDYDKFTIHPEIEGDLRVYSVTYDNEELHIVEEKGD | |
| | LAELLAANLGVEKVDLIRCGGDNLVAAGREQWNDGSNTLTIAPGVVVVYNRNT | |
| | ITNAILESKGLKLIKIHGSELVRGRGGPRCMSMPFEREDI | |
| Enterococcus | MSHPINVFSEIGKLKTVMLHRPGKELENLMPDYLERLLFDDIPFLEKAQAEHD | 19 |
| faecalis | AFAELLRSKDIEVVYLEDLAAEALINEEVRRQFIDQFLEEANIRSESAKEKVR | |
| | ELMLEIDDNEELIQKAIAGIQKQELPKYEQEFLTDMVEADYPFIIDPMPNLYF | |
| | TRDNFATMGHGISLNHMYSVTRQRETIFGQYIFDYHPRFAGKEVPRVYDRSES | |
| | TRIEGGDELILSKEVVAIGISQRTDAASIEKIARNIFEQKLGFKNILAFDIGE | |
| | HRKFMHLDTVFTMIDYDKFTIHPEIEGGLVVYSITEKADGDIQITKEKDTLDN | |
| | ILCKYLHLDNVQLIRCGAGNLTAAAREQWNDGSNTLAIAPGEVVVYDRNTITN | |
| | KALEEAGVKLNYIPGSELVRGRGGPRCMSMPLYREDL | |
| Mycoplasma | MEKKINVFSEIGTLKTVLVHRPGDEIENLTPELLERLLFDDVPFKDVAVKEHD | 20 |
| capricolum | AFTKIMRDNGVEVLYIEKLAAETLDQHPDLREKFIDQFISEANIEDKYKEKYR | |
| | DFISSLDNYRMIKKMIAGTKKLELGIDEGYKAYPFIADPLPNVLFQRDPFSSV | |
| | GFGITMNRMWSVTRNRETIFPDLVFKHHNRFANQVPYYYERDWKEETIEGGDI | |
| | LVLNKETLIIGVTQRTTLKAIEKFSERLFNDPESSYSKVIALDLPKSRAFMHL | |
| | DTVFTNIDYDKFIAHPLIFDCIDEFKIYEVSKQGTKEVKKTLIELLSDAAGRE | |
| | VQIIRCGGNDVVGASREQWNDGTNVVALRPGKVIAYERNWITIDLLRKAGVEV | |
| | LTIASSELSRGRGGPRCMTMPLWREDLQEIKR | |
| Halothermothrix | MFKKSPLNVTSEIGKLKKVLLHRPGHEIENLTPDLLERLLFDDIPYLKVAQEE | 21 |
| orenii | HDAFAQTLRDNGVEVLYLHELAAEAIQEDEIRKKFIEQFLDEAGVIGKGARQV | |
| | LKEYFADMDNETLIRKMMAGVRKKEIPAIEKVASLNDMVEEDYPFVLDPMPNL | |
| | YFTRDPFATIGTGITLNHMRTETRNREVIFAEYIFSYHPDFKDTEIPFWFDRN | |
| | ETTSIEGGDELILSDKVLAMGISERTDAASIEKVARNIFTDGQPFETILAFKI | |
| | PEKRAFMHLDTVFTMVDYDKFTIHAEIEGPLKVYSITKGDNDELKIDEEKATL | |
| | EDTLKKYLGLDEVTLIRCAGGDYIDAGREQWNDGSNTLAIAPGEVVVYNRNHT | |
| | TNRLLEEHGIKLHVIPSSELSRGRGGPRCMSMPLVREDI | |
| Staphylococcus | MTDGPIKVNSEIGALKTVLLKRPGKELENLVPDYLDGLLFDDIPYLEVAQKEH | 22 |
| aureus | DHFAQVLREEGVEVLYLEKLAAESIENPQVRSEFIDDVLAESKKTILGHEEEI | |
| | KALFATLSNQELVDKIMSGVRKEEINPKCTHLVEYMDDKYPFYLDPMPNLYFT | |
| | RDPQASIGHGITINRMFWRARRRESIFIQYIVKHHPRFKDANIPIWLDRDCPF | |
| | NIEGGDELVLSKDVLAIGVSERTSAQAIEKLARRIFENPQATFKKVVAIEIPT | |
| | SRTFMHLDTVFTMIDYDKFTMHSAILKAEGNMNIFIIEYDDVNKDIAIKQSSH | |
| | LKDTLEDVLGIDDIQFIPTGNGDVIDGAREQWNDGSNTLCIRPGVVVTYDRNY | |
| | VSNDLLRQKGIKVIEISGSELVRGRGGPRCMSQPLFREDI | |
| Pseudomonas | MSAEKQKYGVHSEAGKLRKVMVCAPGLAHKRLTPSNCDELLFDDVIWVDQAKR | 23 |
| plecoglossicida | DHFDFVTKMRERGVDVLEMHNLLTDIVQNPEALKWILDRKITPDTVGVGLTNE | |
| | VRSWLEGQEPRHLAEFLIGGVAGQDLPESEGASVVKMYNDYLGHSSFILPPLP | |
| | NTQFTRDTTCWIYGGVTLNPMYWPARRQETLLTTAIYKFHPEFTKADFQVWYG | |
| | DPDQEHGQATLEGGDVMPIGKGIVLIGMGERTSRQAIGQLAQNLFAKGAVEQV | |
| | IVAGLPKSRAAMHLDTVFSFCDRDLVTVFPEVVREIVPFIIRPDESKPYGMDV | |
| | RRENKSFIEVVGEQLGVKLRVVETGGNSFAAEREQWDDGNNVVALEPGVVIGY | |
| | DRNTYTNTLLRKAGIEVITISAGELGRGRGGGHCMTCPIVRDPINY | |
| Pseudomonas | MSAEKQKYGVHSEAGKLRKVMVCAPGLAHKRLTPSNCDELLFDDVIWVDQAKR | 24 |
| putida | DHFDFVTKMRERGVDVLEMHNLLTDIVQNKDALKWILDRKITPDTVGVGLTNE | |
| | VRSWLEGLEPRHLAEFLIGGVAGQDLPQSEGADVVKMYNDYLGHSSFILPPLP | |
| | NTQFTRDTTCWIYGGVTLNPMYWPARRQETLLTTAIYKFHPQFTGADFQVWYG | |
| | DPDKDHGNATLEGGDVMPIGKGIVLIGMGERTSRQAIGQLAQNLFAKGAVEKV | |
| | IVAGLPKSRAAMHLDTVFSFCDRDLVTIFPEVVKEIVPFIIRPDESKPYGMDV | |
| | RRENKSFIEVVGEQLGVKLRVVETGGNSFAAEREQWDDGNNVVAVEPGVVIGY | |
| | DRNTYTNTLLRKAGIEVITISAGELGRGRGGGHCMTCPIVRDPIDY | |
| Pseudomonas | MSTEKTKLGVHSEAGKLRKVMVCSPGLAHQRLTPSNCDELLFDDVIWVNQAKR | 25 |
| aeruginosa | DHFDFVTKMRERGIDVLEMHNLLTETIQNPEALKWILDRKITADSVGLGLTSE | |
| | LRSWLESLEPRKLAEYLIGGVAADDLPASEGANILKMYREYLGHSSFLLPPLP | |
| | NTQFTRDTTCWIYGGVTLNPMYWPARRQETLLTTAIYKFHPEFANAEFEIWYG | |
| | DPDKDHGSSTLEGGDVMPIGNGVVLIGMGERSSRQAIGQVAQSLFAKGAAERV | |
| | IVAGLPKSRAAMHLDTVFSFCDRDLVTVFPEVVKEIVPFSLRPDASSPYGMSI | |
| | RREEKTFLEVVAESLGLKKLRVVETGGNSFAAEREQWDDGNNVVCLEPGVVVG | |
| | YDRNTYTNTLLRKAGVEVITISASELGRGRGGGHCMTCPIIRDPIDY | |
| Mycobacterium | MGVELGSNSEVGALRVVILHRPGAELRRLTPRNTDQLLFDGLPWVSRAQDEHD | 26 |
| tuberculosis | EFAELLASRGAEVLLLSDLLTEALHHSGAARMQGIAAAVDAPRLGLPLAQELS | |
| complex | AYLRSLDPGRLAHVLTAGMTFNELPSDTRTDVSLVLRMHHGGDFVIEPLPNLV | |
| | FTRDSSIWIGPRVVIPSLALRARVREASLTDLIYAHHPRFTGVRRAYESRTAP | |
| | VEGGDVLLLAPGVVAVGVGERTTPAGAEALARSLFDDDLAHTVLAVPIAQQRA | |
| | QMHLDTVCTMVDTDTMVMYANVVDTLEAFTIQRTPDGVTIGDAAPFAEAAAKA | |
| | MGIDKLRVIHTGMDPVVAEREQWDDGNNTLALAPGVVVAYERNVQTNARLQDA | |
| | GIEVLTIAGSELGTGRGGPRCMSCPAARDPL | |
| Mycoplasma | MSVFDSKFKGIHVYSEIGELETVLVHEPGKEIDYITPARLDELLFSAILESHD | 27 |
| arthritidis | ARKEHKEFVAELKKRGINVVELVDLIVETYDLASKEAKEKLLEEFLDDSVPVL | |
| | SDEHRAAVKKFLQSQKSTRSLVEYMIAGITKHDLKIESDLELIVDPMPNLYFT | |
| | RDPFASVGNGVTIHYMRYKVRQRETLFSRFVFSNHPKLVNTPWYYDPAEGLSI | |
| | EGGDVFIYNNDTLVVGVSERTDLQTITLLAKNIKANKECEFKRIVAINVPKWT | |
| | NLMHLDTWLTMLDKDKFLYSPIANDVFKFWDYDLVNGGDAPQPVDNGLPLEDL | |
| | LKSIIGKKPTLIPIAGAGASQIDIERETHFDGTNYLAVAPGIVIGYARNEKTN | |
| | AALEAAGITVLPFRGNQLSLGMGNARCMSMPLSRKDVK | |
| Mycoplasma | MSVFDSKFNGIHVYSEIGELETVLVHEPGREIDYITPARLDELLFSAILESHD | 28 |
| phocicerebrale | ARKEHQSFVKQLKDNGINVVELTDLVAETFDLASKEEQEKLIEEFLEDSEPVL | |
| Artificial full | SEAHKTAVRKFLTSRKSTREMVEFMMAGITKYDLGIEADHELIVDPMPNLYFT | |
| length from new | RDPFASVGNGVTIHYMRYKVRQRETLFSRFVFSNHPKLVKTPWYYDPAMKMSI | |
| species patent. | EGGDVFIYNNDTLVVGVSERTDLETITLLAKNIKANKEVEFKRIVAINVPKWT | |
| | NLMHLDTWLTMLDKDKFLYSPIANDVFKFWDYDLVNGGAEPQPKENGLPLEGL | |
| | LQSIINKKPVLIPIAGNNASHIDIERETHFDGTNYLAIKPGVVIGYARNEKTN | |
| | AALAAAGIKVLPFHGNQLSLGMGNARCMSMPLSRKDVKW | |
| Mycoplasma | MSVFDSKFNGIHVYSEIGELESVLVHEPGREIDYITPARLDELLFSAILESHD | 29 |
| gateae | ARKEHKLFVSELKANDINVVELTDLVTETYDLASQEAKDNLIEEFLEDSEPVL | |
| Artificial full | TEELKSVVRTYLKSIKSTRELIQMMMAGITKYDLGIEADHELIVDPMPNLYFT | |
| length from new | RDPFASVGNGVTIHYMRYKVRQRETLFSRFVFSNHPKLVNTPWYYDPSLKLSI | |
| species patent. | EGGDVFIYNNNTLVVGVSERTDLETVTLLAKNIVANKECEFKRIVAINVPKWT | |
| | NLMHLDTWLTMLDKDKFLYSPIANDVFKFWDYDLVNGGEEPQPVENGLPLEGL | |
| | LESIINKKPILIPIAGEGASQIDIERETHFDGTNYLAIRPGVVIGYSRNEKTN | |
| | AALEAAGIKVLPFHGNQLSLGMGNARCMSMPLSRKDVKW | |
| Mycoplasma | MSVFDSKFNGIHVYSEIGELQTVLVHEPGREIEYITPARLDELLFSAILESHD | 30 |
| phocidae | ARKEHQEFVAELKKNNINVVELTDLVSETYDMVSKEKQEKLIEEFLEDSEPVL | |
| Artificial full | SEEHKGLVRKFLKSLKSSKELIQYMMAGITKHDLNIEADHELIVDPMPNLYFT | |
| length from new | RDPFASVGNGVTIHYMRYKVRQRETLFSRFIFANHPKLMNTPLYYNPDMKLSI | |
| species patent. | EGGDVFVYNNETLVVGVSERTDLDTITLLAKNIKANKEREFKRIVAINVPKWT | |
| | NLMHLDTWLTMLDKDKFLYSPIANDVFKFWDYDLVNGGDEPQPKVNGLPLEKL | |
| | LESIINKKPILIPIAGTSASNIDVERETHFDGTNYLAIAPGVVIGYSRNVKTN | |
| | EALEAAGIKVLPFKGNQLSLGMGNARCMSMPLSRKDVKW | |
| Mycoplasma | MSVFSSKFNGIHVYSEIGELETVLVHEPGKEIDYITPSRLDELLFSAILESHD | 31 |
| salivarium | ARKEHQEFVATLKKEKINVVELTDLVTETYDLVDQKTKDKLIDEFLEDSEPVL | |
| | TAELKATVKKFLKSFKETRKLIEVMMAGITKYDLGIKADRELIVDPMPNLYFT | |
| | RDPFASVGNGVTIHYMRYKVRQRETLFSRFIFNNHPKLVKTPWYYDPAMKMSI | |
| | EGGDVFIYNNDTLVVGVSERTDLDTITLLAKNIKANKECEFKRIVAINVPKWT | |
| | NLMHLDTWLTMLDKDKFLYSPIANDIFKFWDYDLVNGGANPQPKDNGLPLDKL | |
| | LKSIIGKEPVLIPIAGHHATEIEVARETHFDGTNYLAIRPGVVIGYARNEKTN | |
| | EALKDAGITVLPFKGNQLSLGMGNARCMSMPLSRKDVKW | |
| Mycoplasma | MSVFDSKFKGIHVYSEIGELESVLVHEPGREIDYITPARLDELLFSAILESHD | 32 |
| spumans | ARKEHKGFVAELKKQNVNVIELTDLVAETYELASKEAQAKLIEDFIEDSEPVL | |
| | NAEEAQAVRKFLSERKSTREMVEYMMSGLTKYELGLESADRELIVDPMPNLYF | |
| | TRDPFASVGNGVTIHYMKYKVRQRETLFAKFVFSNHPKLVNTPRYYDPSMKLP | |
| | IEGGDVFIYNNETLVVGCSERTELETITLLAKNIKANKEVEFKRIVAINVPKW | |
| | TNLMHLDTWLTMLDKDKFLYSPIANDVFKFWDYDLVNGGEEPQPVENGLPLEE | |
| | LLASIINKKPTLIPIAGEGATHIDVERETHFDGTNYLAIAPALIIGYSRNEKT | |
| | NAALEKAGITVLPFHGNQLSLGMGNARCMSMPLSRKDVKW | |
| Mycoplasma | MSVFDSKFKGIHVYSEIGELETVLVHEPGREIDYITPKRLDELLFSAILESHE | 33 |
| auris | ARKEHKQFVAELKANDINVVELTDLVAETYDLVSQELKDKLIEEFLDDSYPVL | |
| | TEEHKKAVRSFLKSRSSTRELIEYMMAGITKYDLGIEAEGDLIVDPMPNLYFT | |
| | RDPFASVGNGVTIHYMRYKVRQRETLFSRFIFDNHPKLVNTPRYYDPSLKLSI | |
| | EGGDVFIYNNDTLVMGVSERTDLETVTLLAKNIVANKECEFKRIVAINVPHWT | |
| | NLMHLDTWLTMLDKDKFLYSPIANDYFKFWDYDLVNGGAEPQPVVNELPLDKL | |
| | LESIIHKKPILIPIAGEGASQIDLERETHFDGTNYLVLRPGVVVGYARNEKTN | |
| | AALEAVGIKVLPFYGNQLSLGMGNSRCMSMPLSRKDVKW | |
| Mycoplasma | MSVFNSKFKGIHVYSEIGDLESVLVHEPGKEIDYITPSRLDELLFSAILESND | 34 |
| hyosynoviae | ARKEHKEFVEILKKEGVNVVELVDLIAETIDLVDAKKKEALIDEYIEDSEPVV | |
| | DAKVKPLVKKLLLGIKDTKELVKLMMAGITKYDLEIESEKELIIDPMPNLYFT | |
| | RDPFASVGNGVTIHYMRYKVRQRETLFSRFVFRNHPKLTSTPWYYDPAMKLSI | |
| | EGGDVFIYNNDTLVVGVSERTDLDTITLLAKNIKANKECEFKRIVAINVPKWT | |
| | NLMHLDTWLTMLDKDKFLYSPIANDIFKFWDYDLVNGGSEPQPKDNGLPLEKL | |
| | LESIIGKKPVLIPIAGCCASDIEIARETHFDGTNYLAIKPGVVIGYARNEKTN | |
| | KALEKAGIKVLPFKGNQLSLGMGNARCMSMPLSRKDVKW | |
| Mycoplasma | MSVFDKRFKGIHVYSEIGELQTVLVHEPGREIDYITPARLDELLFSAILESHD | 35 |
| cloacale | ARKEHKEFVKILESQGINVVELTDLIAETYELASEEAKDNLIEEFLDESEPVL | |
| | SEEHRILVRNFLKGITKTKELVKMMMAGITKYDLGIEADRELIVDPMPNLYFT | |
| | RDPFASVGNGVTIHYMRYKVRQRETLFSRFIFENHPKLVSTPIYYHPSQGLSI | |
| | EGGDVFIYNNDTLVVGVSERTDLQTITLLAKNIKANEECEFKRIVAINVPKWT | |
| | NLMHLDTWLTMLDKNKFLYSPIANDVFKFWDYDLVNGGDEPQPVDNGLPLNEL | |
| | LASIIGEEPVLVPIAGEGASKMDIERETHFDGTNYLAIAPGVVVGYSRNEKTN | |
| | AALEKAGIKVLPFKGHQLSLGMGNARCMSMPLYRKDVK | |
| Mycoplasma | MSVFDSKFKGIHVYSEIGELESVLVHEPGHEIDYITPSRLDELLFSAMLESHD | 36 |
| alkalescens | ARKEHKQFVAELKANNVNVIELTDLVAETYDLASQEAKDKLIEEFLEDSEPVL | |
| | SEENKIAVRDFLKSRKTTRELIEVMMAGITKYDLGIKNCKCQDLVVDPMPNLY | |
| | FTRDPFASVGNGITIHYMRYKVRQRETLFSRFIFANHPKLVNTPIYYHPSLKL | |
| | SIEGGDVFIYNNDTLVVGVSERTDLETITLLAKNIVANKECEFKRIVAINVPK | |
| | WTNLMHLDTWLTMLDKDKFLYSPIANDVFKFWDYDLVNGGAEPKPVENGSSLE | |
| | AILESIIHKKPILIPIGGDSASQIEVERETHFDGTNYLAIRPGVVIGYSRNVK | |
| | TNAALEAAGIKVIPFHGNQLSLGMGNARCMSMPLSRKDVKW | |
| Mycoplasma | MSKINVYSEIGVLKEVLVHTPGDEIRRIAPSRLDELLFSAILEPSAAIQEHKS | 37 |
| iners | FLKILQDRGIKTIQLSDLVAETYKHYASEAEKEAFIEKYLDEATPVLSKDMRA | |
| | KVKNYILSMQGEPVKMVRTMMAGVSKQELNVESEVELIVDPMPNLYFTRDPFA | |
| | SAGNGISLNNMKYVVRKRETIFAEFIFSIHPEYKKTPHWFDRLDNGSIEGGDV | |
| | FIYNKDTLVIGVSERTNKEAIITIAKHIQDNKEAQFKKIVAINVPPMPNLMHL | |
| | DTWLTMVDKNKFLYSPNMLSVLKVWEIDLSKPIEMVETNKPLAEVLESIIGEK | |
| | PILIPIAGKDATQLDIDIETHFDGTNYLTIAPGVVVGYSRNVKTEAALRAAGV | |
| | TVLSFEGNQLSLGMGSARCMSMPLVREDVK | |
| Mycoplasma | MSKIRVYSEIGNLKKVLVHTPGDEIRRISPSRLEELLFSAVLEPNAAIEEHKR | 38 |
| gallinarum | EVKLLEDRGIQAIQLSDLVAETYVKYATAEQKAAFIEKYLDEATPALSAENRE | |
| | RAKKYILSLEMQPVKMIRTMMAGLSKYELNVESNIELIIDPMPNLYFTRDPFA | |
| | SAGNGISLNNMKYVVRKRETIFAEFIFAIHPEYKETPHWFDRLDHGSIEGGDV | |
| | FVYNKDTLVIGVSERTNKEAIITIAKHIQDNKEAEFKKIVAINVPPMPNLMHL | |
| | DTWLTMVDKNKFIYSPNMLSVLKIWEIDLAKPIEMVESNKSLTEVLESIIGEK | |
| | PILIPIAGEGASQLDIDIETHFDGTNYLTIAPGVVVGYSRNEKTEKALKAAGI | |
| | TVLSFEGNQLSLGMGSARCMSMPLVREDVK | |
| Mycoplasma | MNSNQKGIHVYSEIGKLKEVLVHRPGRELDFLDPTRLDELLFAATLEAETARL | 39 |
| pirum | EHDNFTNALKNQGVTVIELADLVAQTYSSSTPTIKAAFINKYLDEATPALTTK | |
| | LRTLVKDFLTKQKSVRKMVDYMIGGILSTDLNIKGKPELIVEPMPNAYFTHDP | |
| | FASVGNGVTLHYMKHNVRRREVLFSEFIFNNNERFQNTPRYIVPTKGLDIEGG | |
| | DVFVYNKNTLVVGVSERTKMVTIKELAKNILKNKECLFKKIYAINVPKMPNLM | |
| | HLDTWLTMLDHNKFLYSPNMLSVLKIWEIDISSGKSISSPKELNMDLSKALSI | |
| | IIGKKPILIPVAGENASQIDINIETNFDATNYLVTQPGVVVGYSRNKKTEAAL | |
| | IKAGIEVIPFQGNQLSLGMGSARCMSMPLIREDV | |
| Mycoplasma | MSKSKINVYSEYGNLKEVLVHTPGDEIRRITPSRLDELLFSAILEPKSAIAEH | 40 |
| primatum | KSFCQILKDNKVKAIQLDELVAATYKGVSESVQNSFVERWLDECEPKLENNVR | |
| | PIVKEYLLKAAEQSVKKMIRIMMAGIDKREIGVESEVDFIVDPMPNLYFTRDP | |
| | FASVGNGITLHHMKYVVRQRETLFSEFIFDNHPDYKFVPRYFDRDDEGKIEGG | |
| | DVFIYNSKTLVVGISERTNKDAIRIVAKKIQANADAKFEKIFAINVPPMPNLM | |
| | HLDTWLTMLDSNKFLYSPNMLSVLKVWEINLDDPALEWKEISGSLEEILTYII | |
| | GKKPILIPIAGKGASQFEIDIETHFDGTNYLAIAPSVVIGYSRNELTEKALKK | |
| | AGVKVLSLDGNQLSLGMGSARCMSMPLIREDVK | |
| Mycoplasma | MSKINVYSEVGVLKEVLVHTPGDEIRRVAPSRLDELLFSAILEPQDAIAEHKR | 41 |
| lipofaciens | FIKILEDNNIKVIQLDELVSETWEKATAEQRDAFIEKWLDEAEPVLDAKLRET | |
| | VKKYLLSLNPVKKMVRTMMAGIDKKELKIELDRDLVVDPMPNLYFTRDPFASA | |
| | GNGISLNNMKYVTRKRETIFAEFIFNIHPDYKTTPHWFDRLDKGNIEGGDVFI | |
| | YNKDTLVLGVSERTNKDAVMTIAKHIQSNEQAKFKKLVAINVPPMPNLMHLDT | |
| | WLTMVDHDKFLYSPNMLSVLKIWEIDLTPGKEIEMVESTKSLSDMLESIIGKK | |
| | PVLIPIAGKDASQLDIDIETHFDGTNYLTIRPGVVVGYSRNCLTEQALKDAGV | |
| | TVLSFDGNQLSLGMGSARCMSMPLVREDIK | |
| Mycoplasma | MNKINVYSEIGKLKEVLVHTPGNEIRRISPSRLDELLFSALLEPNFAAKEHTA | 42 |
| felifaucium | FCEILKENGIKAIQLVDLVSDTWRIASEKAKTEFIERWLDECEPKLDSNLREI | |
| | VRKHIYAIEKRSVKRMVKTMMAGIERRELPVTSKEVARELVVDPMPNLYFTRD | |
| | PFASVGNGISLHHMKYVTRQRETIFAEFVFGNHPDYIDTPRWFDRSDDGRIEG | |
| | GDVFIYGSKTLVIGVSERTNKEAIKVMAKKIQANKEATFEKIYAINVPPMPNL | |
| | MHLDTWLTMLDKNKFLYSPNMLAVLQVWEIDLKDPELTWHELSGSLEEILHKI | |
| | IGRKPILIPIAGHGAQQIDIDIETHFDGTNYLAIAPGVVVGYNRNVLTERALK | |
| | KAGIKVLSFEGNQLSLGMGSARCMSMPLIRENLK | |
| Mycoplasma | MFNKIKVYSEIGRLRKVLVHTPGKELEYVTPQRLDELLFSSLLNPVKARQEHE | 43 |
| imitans | AFIKILQDQGVECVQLTTLTAQTFQSATSEVKEKFINRWLDECLPKLSDDNRI | |
| | KVYAYLKDLSSDPEVMIRKMMSGILAKEVNVQSDVELIADPMPNLYFTRDPFA | |
| | SIGKGVTLHSMFHPTRKRETIFADFVFSHHPEYKQTPKYYSRLNEYSIEGGDL | |
| | FVYDDKTLVIGVSERTEKKAIQFLAEKLRENYETTFEKIYAINVPKMSNLMHL | |
| | DTWLTMLDYDKFLYSPNMMGVLKIWEIDLTHEQLSWRELNESLEEFLSMVIGK | |
| | KATTIPVAGEDSTQIEIDVETNFDATNFLVIQPGVVVGYDRNYKTNQALVNAG | |
| | IKVLSWNGDQLSLGMGSARCMSMPLYRDPIKKG | |
| Mycoplasma | MSKINVYSEIGTLKEVLVHTPGDEIRRVAPARLDELLFSAILEPNHAIAEHKA | 44 |
| opalescens | FIKILEDNGIKVIQLDELVVQTWNQVDEATRKAFVTKWLDECEPKLESNVRVE | |
| | VEKYIYSLAKEPKKMVRTMMAGISKEELPLNVNRPLVVDPMPNLYFTRDPFAS | |
| | VGTGISLHHMKYVTRQRETIFAQFVFDNHKDYNTVPRWFDNKDQGRIEGGDVF | |
| | IYNTKTLVIGVSERTDKDAIKIMAKKIQADKNCKFEKIFAINVPPMPNLMHLD | |
| | TWLTMVDRNKFLYSPNMLSVLKVWEIDLKDASLAWKEIEGSLSQILEKIIGEK | |
| | PILIPIAGENASQLDIDIETHFDGTNYLTIAPGVVVGYSRNVKTEQALKAAGV | |
| | KVLSFEGNQLSLGMGSARCMSMPLIREDLK | |
| Mycoplasma | MKKNAINVYSEIGKLKKVLVHRPGDELKYVTPQRMDELLMSAIIELEQAKEEH | 45 |
| moatsii | DAFTKILRDNGVEVIELADLTAEMYDSLTPSEKDAFLNQWVKEASWGKKSSID | |
| | ALKIKKNLSKKVFDYVKSIKPTRKMIDKLMAGVLLSEIGEKSIILNKDKKNEM | |
| | VIDLVVDPMPNLYFTRDPFASVGNGITLHNMKYPTRKRETIFAQWIFNKHPEY | |
| | KDVPQFISKRDGKETIEGGDVFIYTKDVLAIGVSERTNMEAILRIATNIKKDK | |
| | NCEFKKIVAINVPPMGNLMHLDTWLTMLDKDLFLYSGNIKSALKVWEIDLTKP | |
| | ITPKSPKLSTAKLADILAKIVGKKVRMIPIGGKDGNQMDIDIETHFDGTNYLA | |
| | IAPGVVVGYHRNRKTQKALEEAGVKVLAFQGNQLSLGMGSARCMSMPLVREEV | |
| | K | |
| Mycoplasma | MSQINVFSEIGQLKEVLVHTPGDEIRRISPKRYNELLFSAILEADVAIKEHKS | 46 |
| elephantis | FVKILEENNVKVIQLKDILLETWNICSKEAKNIFINKWIEEAQPVIHSSSLKE | |
| | KIKLFLKSKTPLEIIDIMMKGILKQELGIEYKHELIIDPMPNLYFTRDPFTSM | |
| | GSGITINNMKYQTRKRETIFSEFIFNNHPKYKNTPRWFDRFDSGNIEGGDLFV | |
| | YTKETIVVGVSERTKKKAILKIAKNIQENNNSFKKIVVIKVPIMQNLMHLDTW | |
| | IVMVDFDKFIYSPNVTKSLKFWEIDLTKKPKFIQLKNETLEDVLYRVIGKKPI | |
| | LIPVAGENANQIDIDVETHFDATNYLTIRPGVVVGYSRNKKTEEALINAGVKV | |
| | YAFEGNQLSLGMGSARCMSMPLIREDII | |
| Mycoplasma | MKNINVYSEVGKLKEVVVHTPGEELHNVAPSRLQELLTSAVLEPEVARKEHLK | 47 |
| testudinis | FIKILNDYGVKVIQIVDLITETYEAVDSNKKEAFINNWLDNSVPKLTDKNRMI | |
| | LRNYLTQFSTKAMIRKMISGIRAKELNLKTPSALLVDPMPNLCFARDTFACVG | |
| | SAISLSTMKHPTRRREALLTEFIFQNHPKYKDVIKYFDSKNSKATIEGGDIFV | |
| | YNPKTLVVGNSERTNMQACLLLAKKIQSNPNNKFEKIVIVNVPPLPHLMHLDT | |
| | WLTMVDYDKFIYSPNILHTLKFWVIDLKKRKLEAVEKHNTLKAMLRMIIKKEP | |
| | ILIPVGDVGADQLDIDLETHFDATNYLALAPGVVVGYDRNIKTQRALEKAGVK | |
| | VLSFSGNQLSLAMGSARCLSMPLIREEN | |
| Mycoplasma | MSVFDSKFKGIHVYSEIGELESVLVHEPGREIDYITPARLDELLFSAILESHD | 48 |
| canadense | ARKEHKQFVSELKANDINVVELTDLVAETYDLASQEAKDKLIEEFLEDSEPVL | |
| | SEEHKAIVRKYLKGIQPTRKLIEMMMAGITKYDLGIEADHELIVDPMPNLYFT | |
| | RDPFASVGNGVTIHYMRYKVRQRETLFSRFVFSNHPKLVNTPWYYDPSLKLSI | |
| | EGGDVFVYNNDTLVVGVSERTDLQTVTLLAKNIVANKECEFKRIVAINVPKWT | |
| | NLMHLDTWLTMLDKDKFLYSPIANDVFKFWDYDLVNGGSEPQPVENGLPLEGL | |
| | LESIINKKPILIPIAGEGASQMEIERETHFDGTNYLAIRPGVVIGYSRNEKTN | |
| | AALEAAGIKVLPFHGNQLSLGMGNARCMSMPLSRKDVKW | |
| Mycoplasma | MSVFDKRFKGIHVYSEIGELQTVLVHEPGREIDYITPARLDELLFSAILESHD | 49 |
| anseris | ARAEHKKFVATLKEQGINTVELTDLVAETYDLASQEARDNLLEEFLDDSAPVL | |
| | SEEHKEIVRTYLKGIKGTRKLIETMMAGITKYDLGIEAEQELIVDPMPNLYFT | |
| | RDPFASVGNGVTIHYMRYKVRQRETLFSRFIFSNHPQLVNTPWYYNPAEGLSI | |
| | EGGDVFIYNNDTLVVGVSERTDLQTITLLAKNIKANEECEFKRIVAINVPKWT | |
| | NLMHLDTWLTMLDTNKFLYSPIANDVFKFWDYDLVNGGDEPQPVDNGLPLNEL | |
| | LKSIIGEEPILIPIAGDGATQIEIERETHFDGTNYLAIAPGVVIGYSRNEKTN | |
| | AALEAAGIKVLPFKGHQLSLGMGNARCMSMPLYRKDVK | |
| Mycoplasma | MSKINVYSEIGVLKEVLVHTPGDEIRRISPSRLDELLFSAILQPEQAIKEHQS | 50 |
| meleagridis | FVKILQDRGIKVIQLSDLVAETYVKYATSKEKESFIEKWLDEATPALNSENRA | |
| | RVKNYITAMQGQPVKMVRAMMAGVSKQELNIESDVELIVDPMPNLYFTRDPFA | |
| | SAGNGISLNNMKYVVRKRETIFAEFIFSIHPEYKQTPHWFDRLDKGNIEGGDV | |
| | FIYNKDTLVIGVSERTNKEAILTIAEHIKNNKEAKFKKIVAINVPPMPNLMHL | |
| | DTWLTMVDKNKFLYSPNMLSVLKIWEIDLSKEIKMVETSKPLADVLESIIGEK | |
| | PILIPIAGENASQLDIDIETHFDGTNYLTIAPGVVVGYSRNVKTEAALKAAGV | |
| | TVYSFDGNQLSLGMGSGRCMSMPLVREDVK | |
| Mycoplasma | MSIKENGIHVYSEIGKLRDVLVHRPGRELNFLDPSRLDELLFAATLEPETARL | 51 |
| alvi | EHDNFTTVLKNQGVNVIELADLVSQTYSKVDSKVKKEFIDQYLNEATPKLTSE | |
| | LSKKVYDFLTKQKSNREMVDFMMGGILSSDLNIKGQPYLIVEPMPNLYFTRDP | |
| | FASVGNGATIHWMKHNVRRREVLFANFIFKYNERFQNTPKYITPTKGLDIEGG | |
| | DVFVYNKKTLVVGVSERTKMETIKELAKNISKNKECTFTKIYAINVPKMPNLM | |
| | HLDTWLTMLDYNKFLYSPNMLSVLKVWEINISNNKVSAPKELNVNLEKALSMI | |
| | IGKKPILIPVAGANASQIDINIETNFDATNYLVIEPGVVVGYSRNKKTEEALV | |
| | KAGIKVLPFHGNQLSLGMGSARCMSMPLYREDV | |
| Mycoplasma | MSSIDKNSLGNGINVYSEIGELKEVLVHTPGDEIRYTAPSRLEELLFSAVLKA | 52 |
| penetrans | DTAIEEHKGFVKILQNNGIKVIQLCDLVAETYELCSKEVRNSFIEQYLDEALP | |
| | VLKKEIRPVVKDYLLSFPTVQMVRKMMSGILANELNIKQDNPLIIDGMPNLYF | |
| | TRDPFASMGNGVSINCMKYPTRKREVIFSRFVFTNNPKYKNTPRYFDIVGNNG | |
| | TIEGGDIFIYNSKTLVIGNSERTNFAAIESVAKNIQANKDCTFERIVVINVPP | |
| | MPNLMHLDTWLTMLDYDKFLYSPNMMNVLKIWEIDLNVKPVKFVEKKGTLEEV | |
| | LYSIIDKKPILIPIAGKGANQLDIDIETHFDGTNYLTIAPGVVVGYERNEKTQ | |
| | KALVEAGIKVLSFNGSQLSLGMGSARCMSMPLIRENLKK | |
| Mycoplasma | MKKINVYSEYGKLKEVLVHTPGDEIRRIAPSRLDELLFSAILEPDSAIAEHKR | 53 |
| fermentans | FVQLLKDNGIKVIQLDELFAKTFDLVSESVKQSFIERWLDECEPKLDATLRAK | |
| | VKEYILELKAKSSKKHVRVMMAGIDKKELGIELDRDLVVDPMPNLYFTRDPFA | |
| | SVGNGISLHHMKYVTRQRETIFSEFIFDNNLDYNTVPRWFDRKDEGRIEGGDV | |
| | FIYSADTLVVGVSERTNKEAINVMARKIAADKEVKFKRIYAINVPPMPNLMHL | |
| | DTWLTMLDKNKFLYSPNMLSVLKVWRIDLNDPDFVWHEIEGSLEEILEQIIGM | |
| | KPILIPIAGKGASQLDIDIETHFDGTNYLTIAPSVVVGYSRNEKTEKALKAAK | |
| | VKVLSFEGNQLSLGMGSARCMSMPLIREDIKKK | |
| Mycoplasma | MKYNINVHSEIGQLQTVLVHTPGNEIRRISPRRLDDLLFSAVIEPDTAIQEHQ | 54 |
| pneumoniae | TFCQLLQEQNIEVVQLTDLTATTFDKANATAQNQFIETWLDQAEPKLTPEHRK | |
| | VAKQYLLEQKAKSTLSMVRSMMGGIDKRKVAAANTINGDFLVDPMPNLYFTRD | |
| | PFASIGHGISINRMKYLTRRRETLFASFIFANHPIIAARKFYFKPIDMGTIEG | |
| | GDIFVYDQQTVVMGLSERTTEAAINVLAKKIQQDSSTSFKRIFVINVPQLPNL | |
| | MHLDTWLTMLDRNKFLYSPNMLAVLKAWRIDFTDPALKWNEIAGDLSTILHTI | |
| | IGQKPMLIPIAGADANQTEIDIETHFDGTNYLTIAPSVVVGYARNKLTHQTLE | |
| | AAGVKVIAFKGNQLSLGMGSARCMSMPLVRKPL | |
| Mycoplasma sp. | MEKIHVTSEIGPLKKVLLHRPGNELLNLTPDTLSRLLFDDIPYLPDAIKEHDE | 55 |
| CAG: 877 | FADALRANGVEVVYLENLMADVLDLSDEIRDKFIKQFIYEAGIRTPKYKYLVF | |
| | DYLDQITNSKKLVLKTMEGIQISDIPRRKREIEKSLVDLIETEDEFIADPMPN | |
| | LYFTRDPFASVGEGISLNKMYSVTRNRETIYAEYIFKYHPDYKDQARLYYDRY | |
| | NPYHIEGGDVLNINDHVLAIGISQRTTAEAIDQIAKNLFKDPECKIDTILAFN | |
| | IPESRAFMHLDTVFTQVDYDKFTYHPGIMGTLQVFEITEGDDPNSDEDLTVTE | |
| | INAPLEEILTKYVGRKVTLIPCAGGDKVSAEREQWNDGSNTLCIAPGVVVVYD | |
| | RNNLTNAVLRSYGLKVIEIHGAELSRGRGGPRCMSMPLVREDI | |
| Mycoplasma sp. | MHVTSEIKKLKKVLVHRPGKELLNLTPDTLGRLLFDDIPYLKDAILEHDEFCQ | 56 |
| CAG: 472 | ILRDNDVEVVYLEDLMAETLDENPQVKPSFIRQFIYEAGVRTPKYKDLLFDYL | |
| | MSYTNNKELVLKTMEGIKVSEVHRNKQDSEYSLVDQISEETKFLAEPMPNLYF | |
| | TRDPFASVGDGIILNKMHSVTRSRETIYAYYIFNYHPDYMDKVPKYYDRENPF | |
| | SIEGGDVLNLNEHTLAIGISQRTSAEAIDLVAKNMFNDEKCNIDTILAFKIPE | |
| | CRAFMHLDTVFTQIDIDKFTYHPGIMDTLEVFEITKNEDDLDEVRVIKKEGSL | |
| | ENILEEYLGIDITLIPCAGGDKIASEREQWNDGTNTLCIAPGVVVVYNRNNIT | |
| | NEVLREKGIKVIEMNSAELSRGRGGPRCMSMPLERED | |

Hence, in some embodiments, the ADI polypeptide in the ADI-PEG comprises, consists, or consists essentially of an illustrative sequence from Table A1 (SEQ ID NOs:1-56), or a variant or fragment thereof having ADI activity.

Certain embodiments include variants of the reference ADI polypeptide sequences described herein, whether described by name or by reference to a Table or sequence identifier (e.g., Table A1, SEQ ID NOs:1-56). A “variant” sequence refers to a polypeptide or polynucleotide sequence that differs from a reference sequence by one or more substitutions, deletions (e.g., truncations), additions, and/or insertions. Certain variants thus include fragments of a reference sequence described herein. Variant polypeptides are biologically active, that is, they continue to possess the enzymatic or binding activity of a reference polypeptide. Such variants may result from, for example, genetic polymorphism and/or from human manipulation.

In many instances, a biologically active variant will contain one or more conservative substitutions. A “conservative substitution” is one in which an amino acid is substituted for another amino acid that has similar properties, such that one skilled in the art of peptide chemistry would expect the secondary structure and hydropathic nature of the polypeptide to be substantially unchanged. As described above, modifications may be made in the structure of the polynucleotides and polypeptides of the present disclosure and still obtain a functional molecule that encodes a variant or derivative polypeptide with desirable characteristics. When it is desired to alter the amino acid sequence of a polypeptide to create an equivalent, or even an improved, variant or portion of a polypeptide described herein, one skilled in the art will typically change one or more of the codons of the encoding DNA sequence.

For example, certain amino acids may be substituted for other amino acids in a protein structure without appreciable loss of interactive binding capacity with structures such as, for example, antigen-binding regions of antibodies or binding sites on substrate molecules. Since it is the interactive capacity and nature of a protein that defines that protein's biological functional activity, certain amino acid sequence substitutions can be made in a protein sequence, and, of course, its underlying DNA coding sequence, and nevertheless obtain a protein with like properties. It is thus contemplated that various changes may be made in the peptide sequences of the disclosed compositions, or corresponding DNA sequences which encode said peptides without appreciable loss of their utility.

In making such changes, the hydropathic index of amino acids may be considered. The importance of the hydropathic amino acid index in conferring interactive biologic function on a protein is generally understood in the art (Kyte & Doolittle, 1982, incorporated herein by reference). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Each amino acid has been assigned a hydropathic index on the basis of its hydrophobicity and charge characteristics (Kyte & Doolittle, 1982). These values are: isoleucine (±4.5); valine (±4.2); leucine (±3.8); phenylalanine (±2.8); cysteine (±2.5); methionine (±1.9); alanine (±1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5). It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those within ±1 are particularly preferred, and those within ±0.5 are even more particularly preferred.

It is also understood in the art that the substitution of like amino acids can be made effectively on the basis of hydrophilicity. U.S. Pat. No. 4,554,101 (specifically incorporated herein by reference in its entirety), states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: arginine (±3.0); lysine (±3.0); aspartate (±3.0±1); glutamate (±3.0±1); serine (±0.3); asparagine (±0.2); glutamine (±0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those within ±1 are particularly preferred, and those within ±0.5 are even more particularly preferred.

As outlined above, amino acid substitutions are generally therefore based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include: arginine and lysine; glutamate and aspartate; serine and threonine; glutamine and asparagine; and valine, leucine and isoleucine.

Amino acid substitutions may further be made on the basis of similarity in polarity, charge, solubility, hydrophobicity, hydrophilicity and/or the amphipathic nature of the residues. For example, negatively charged amino acids include aspartic acid and glutamic acid; positively charged amino acids include lysine and arginine; and amino acids with uncharged polar head groups having similar hydrophilicity values include leucine, isoleucine and valine; glycine and alanine; asparagine and glutamine; and serine, threonine, phenylalanine and tyrosine. Other groups of amino acids that may represent conservative changes include: (1) ala, pro, gly, glu, asp, gln, asn, ser, thr; (2) cys, ser, tyr, thr; (3) val, ile, leu, met, ala, phe; (4) lys, arg, his; and (5) phe, tyr, trp, his.

A variant may also, or alternatively, contain non-conservative changes. In a preferred embodiment, variant polypeptides differ from a native or reference sequence by substitution, deletion or addition of fewer than about 10, 9, 8, 7, 6, 5, 4, 3, 2 amino acids, or even 1 amino acid. Variants may also (or alternatively) be modified by, for example, the deletion or addition of amino acids that have minimal influence on the immunogenicity, secondary structure, enzymatic activity, and/or hydropathic nature of the polypeptide.

In certain embodiments, a polypeptide sequence is about, at least about, or up to about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 800, 810, 820, 830, 840, 850, 860, 870, 880, 890, 900, 900, 910, 920, 930, 940, 950, 960, 970, 980, 990, 1000 or more contiguous amino acids in length, including all integers in between, and which may comprise all or a portion of a reference sequence (see, e.g., Sequence Listing).

In some embodiments, a polypeptide sequence consists of about or no more than about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800. 800, 810, 820, 830, 840, 850, 860, 870, 880, 890, 900, 900, 910, 920, 930, 940, 950, 960, 970, 980, 990, 1000 or more contiguous amino acids, including all integers in between, and which may comprise all or a portion of a reference sequence (see, e.g., Sequence Listing).

In certain embodiments, a polypeptide sequence is about 10-1000, 10-900, 10-800, 10-700, 10-600, 10-500, 10-400, 10-300, 10-200, 10-100, 10-50, 10-40, 10-30, 10-20, 20-1000, 20-900, 20-800, 20-700, 20-600, 20-500, 20-400, 20-300, 20-200, 20-100, 20-50, 20-40, 20-30, 50-1000, 50-900, 50-800, 50-700, 50-600, 50-500, 50-400, 50-300, 50-200, 50-100, 100-1000, 100-900, 100-800, 100-700, 100-600, 100-500, 100-400, 100-300, 100-200, 200-1000, 200-900, 200-800, 200-700, 200-600, 200-500, 200-400, or 200-300 contiguous amino acids, including all ranges in between, and comprises all or a portion of a reference sequence. In certain embodiments, the C-terminal or N-terminal region of any reference polypeptide may be truncated by about 1, 2, 3, 4, 5, 6, 7, 8, 9, 10, 15, 20, 25, 30, 35, 40, 45, 50, 60, 70, 80, 90, 100, 110, 120, 130, 140, 150, 160, 170, 180, 190, 200, 250, 300, 350, 400, 450, 500, 550, 600, 650, 700, 750, or 800 or more amino acids, or by about 10-50, 20-50, 50-100, 100-150, 150-200, 200-250, 250-300, 300-350, 350-400, 400-450, 450-500, 500-550, 550-600, 600-650, 650-700, 700-750, 750-800 or more amino acids, including all integers and ranges in between (e.g., 101, 102, 103, 104, 105), so long as the truncated polypeptide retains the binding properties and/or activity of the reference polypeptide. Typically, the biologically-active fragment has no less than about 1%, about 5%, about 10%, about 25%, or about 50% of an activity of the biologically-active reference polypeptide from which it is derived.

In general, variants will display at least about 30%, 40%, 50%, 55%, 60%, 65%, 70%, 75%, 80%, 85%, 90%, 91%, 92%, 93%, 94%, 95%, 96%, 97%, 98%, 99% similarity or sequence identity or sequence homology to a reference polypeptide sequence. Moreover, sequences differing from the native or parent sequences by the addition (e.g., C-terminal addition, N-terminal addition, both), deletion, truncation, insertion, or substitution (e.g., conservative substitution) of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, or 100 amino acids (including all integers and ranges in between) but which retain the properties or activities of a parent or reference polypeptide sequence are contemplated.

In some embodiments, variant polypeptides differ from reference sequence by at least one but by less than 50, 40, 30, 20, 15, 10, 8, 6, 5, 4, 3 or 2 amino acid residue(s). In certain embodiments, variant polypeptides differ from a reference sequence by at least 1% but less than 20%, 15%, 10% or 5% of the residues. (If this comparison requires alignment, the sequences should be aligned for maximum similarity. “Looped” out sequences from deletions or insertions, or mismatches, are considered differences.) Calculations of sequence similarity or sequence identity between sequences (the terms are used interchangeably herein) are performed as follows. To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In certain embodiments, the length of a reference sequence aligned for comparison purposes is at least 30%, preferably at least 40%, more preferably at least 50%, 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position.

The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch, (J. Mol. Biol. 48: 444-453, 1970) algorithm which has been incorporated into the GAP program in the GCG software package, using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package, using a NWSgapdna.CMP matrix and a gap weight of 40, 50, 60, 70, or 80 and a length weight of 1, 2, 3, 4, 5, or 6. A particularly preferred set of parameters (and the one that should be used unless otherwise specified) are a Blossum 62 scoring matrix with a gap penalty of 12, a gap extend penalty of 4, and a frameshift gap penalty of 5.

The percent identity between two amino acid or nucleotide sequences can be determined using the algorithm of E. Meyers and W. Miller (Cabios. 4:11-17, 1989) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al., (1990, J. Mol. Biol, 215: 403-10). BLAST nucleotide searches can be performed with the NBLAST program, score=100, wordlength=12 to obtain nucleotide sequences homologous to nucleic acid molecules described herein. BLAST protein searches can be performed with the XBLAST program, score=50, wordlength=3 to obtain amino acid sequences homologous to protein molecules described herein. To obtain gapped alignments for comparison purposes, Gapped BLAST can be utilized as described in Altschul et al., (Nucleic Acids Res. 25: 3389-3402, 1997). When utilizing BLAST and Gapped BLAST programs, the default parameters of the respective programs (e.g., XBLAST and NBLAST) can be used.

In some embodiments, as noted above, polynucleotides and/or polypeptides can be evaluated using a BLAST alignment tool. A local alignment consists simply of a pair of sequence segments, one from each of the sequences being compared. A modification of Smith-Waterman or Sellers algorithms will find all segment pairs whose scores cannot be improved by extension or trimming, called high-scoring segment pairs (HSPs). The results of the BLAST alignments include statistical measures to indicate the likelihood that the BLAST score can be expected from chance alone.

The raw score, S, is calculated from the number of gaps and substitutions associated with each aligned sequence wherein higher similarity scores indicate a more significant alignment. Substitution scores are given by a look-up table (see PAM, BLOSUM).

Gap scores are typically calculated as the sum of G, the gap opening penalty and L, the gap extension penalty. For a gap of length n, the gap cost would be G+Ln. The choice of gap costs, G and L is empirical, but it is customary to choose a high value for G (10-15), e.g., 11, and a low value for L (1-2) e.g., 1.

The bit score, S′, is derived from the raw alignment score S in which the statistical properties of the scoring system used have been taken into account. Bit scores are normalized with respect to the scoring system, therefore they can be used to compare alignment scores from different searches. The terms “bit score” and “similarity score” are used interchangeably. The bit score gives an indication of how good the alignment is; the higher the score, the better the alignment.

The E-Value, or expected value, describes the likelihood that a sequence with a similar score will occur in the database by chance. It is a prediction of the number of different alignments with scores equivalent to or better than S that are expected to occur in a database search by chance. The smaller the E-Value, the more significant the alignment. For example, an alignment having an E value of e⁻¹¹⁷ means that a sequence with a similar score is very unlikely to occur simply by chance. Additionally, the expected score for aligning a random pair of amino acids is required to be negative, otherwise long alignments would tend to have high score independently of whether the segments aligned were related. Additionally, the BLAST algorithm uses an appropriate substitution matrix, nucleotide or amino acid and for gapped alignments uses gap creation and extension penalties. For example, BLAST alignment and comparison of polypeptide sequences are typically done using the BLOSUM62 matrix, a gap existence penalty of 11 and a gap extension penalty of 1.

In some embodiments, sequence similarity scores are reported from BLAST analyses done using the BLOSUM62 matrix, a gap existence penalty of 11 and a gap extension penalty of 1.

In a particular embodiment, sequence identity/similarity scores provided herein refer to the value obtained using GAP Version 10 (GCG, Accelrys, San Diego, Calif.) using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix (Henikoff and Henikoff, PNAS USA. 89:10915-10919, 1992). GAP uses the algorithm of Needleman and Wunsch (J Mol Biol. 48:443-453, 1970) to find the alignment of two complete sequences that maximizes the number of matches and minimizes the number of gaps.

In particular embodiments, the variant polypeptide comprises an amino acid sequence that can be optimally aligned with a reference polypeptide sequence (see, e.g., Sequence Listing) to generate a BLAST bit scores or sequence similarity scores of at least about 50, 60, 70, 80, 90, 100, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, or more, including all integers and ranges in between, wherein the BLAST alignment used the BLOSUM62 matrix, a gap existence penalty of 11, and a gap extension penalty of 1.

As noted above, a reference polypeptide may be altered in various ways including amino acid substitutions, deletions, truncations, additions, and insertions. Methods for such manipulations are generally known in the art. For example, amino acid sequence variants of a reference polypeptide can be prepared by mutations in the DNA. Methods for mutagenesis and nucleotide sequence alterations are well known in the art. See, for example, Kunkel (PNAS USA. 82: 488-492, 1985); Kunkel et al., (Methods in Enzymol. 154: 367-382, 1987), U.S. Pat. No. 4,873,192, Watson, J. D. et al., (“Molecular Biology of the Gene,” Fourth Edition, Benjamin/Cummings, Menlo Park, Calif., 1987) and the references cited therein. Guidance as to appropriate amino acid substitutions that do not affect biological activity of the protein of interest may be found in the model of Dayhoff et al., (1978) Atlas of Protein Sequence and Structure (Natl. Biomed. Res. Found., Washington, D.C.).

Methods for screening gene products of combinatorial libraries made by such modifications, and for screening cDNA libraries for gene products having a selected property are known in the art. Such methods are adaptable for rapid screening of the gene libraries generated by combinatorial mutagenesis of reference polypeptides. As one example, recursive ensemble mutagenesis (REM), a technique which enhances the frequency of functional mutants in the libraries, can be used in combination with the screening assays to identify polypeptide variants (Arkin and Yourvan, PNAS USA 89: 7811-7815, 1992; Delgrave et al., Protein Engineering. 6: 327-331, 1993).

“Polyethylene glycol” or “PEG” refers to mixtures of condensation polymers of ethylene oxide and water, in a branched or straight chain, represented by the general formula H(OCH₂CH₂)ₙOH, wherein n is at least 4. “Polyethylene glycol” or “PEG” is used in combination with a numeric suffix to indicate the approximate weight average molecular weight thereof. For example, PEG5,000 refers to PEG having a total weight average molecular weight of about 5,000; PEG12,000 refers to PEG having a total weight average molecular weight of about 12,000; and PEG20,000 refers to PEG having a total weight average molecular weight of about 20,000.

In some embodiments, the PEG has a total weight average molecular weight of about 1,000 to about 50,000; about 3,000 to about 40,000; about 5,000 to about 30,000; about 8,000 to about 30,000; about 11,000 to about 30,000; about 12,000 to about 28,000; about 16,000 to about 24,000; about 18,000 to about 22,000; or about 19,000 to about 21,000. In some embodiments, the PEG has a total weight average molecular weight of about 1,000 to about 50,000; about 3,000 to about 30,000; about 3,000 to about 20,000; about 4,000 to about 12,000; about 4,000 to about 10,000; about 4,000 to about 8,000; about 4,000 to about 6,000; or about 5,000. In specific embodiments, the PEG has a total weight average molecular weight of about 20,000. Generally, PEG with a molecular weight of 30,000 or more is difficult to dissolve, and yields of the formulated product may be reduced. The PEG may be a branched or straight chain. Generally, increasing the molecular weight of the PEG decreases the immunogenicity of the ADI. The PEG may be a branched or straight chain, and in certain embodiments is a straight chain. The PEG having a molecular weight described herein may be used in conjunction with ADI, and optionally, a biocompatible linker.

Certain embodiments employ thiol, sulfhydryl, or cysteine-reactive PEG(s). In some embodiments, the thiol, sulfhydryl, or cysteine-reactive PEG(s) are attached to one or more naturally-occurring cysteine residues, one or more introduced cysteine residues (e.g., substitution of one or more wild-type residues with cysteine residue(s)), insertion of one or more cysteine residues), or any combination thereof (see, e.g., Doherty et al., Bioconjug Chem. 16:1291-98, 2005). In certain embodiments, certain of the wild-type ADI cysteines residues may be first substituted with another amino acid to prevent attachment of the PEG polymer to wild-type cysteines, for example, to prevent the PEG(s) from disrupting an otherwise desirable biological activity. Some embodiments employ one or more non-natural cysteine derivatives (e.g., homocysteine) instead of cysteine.

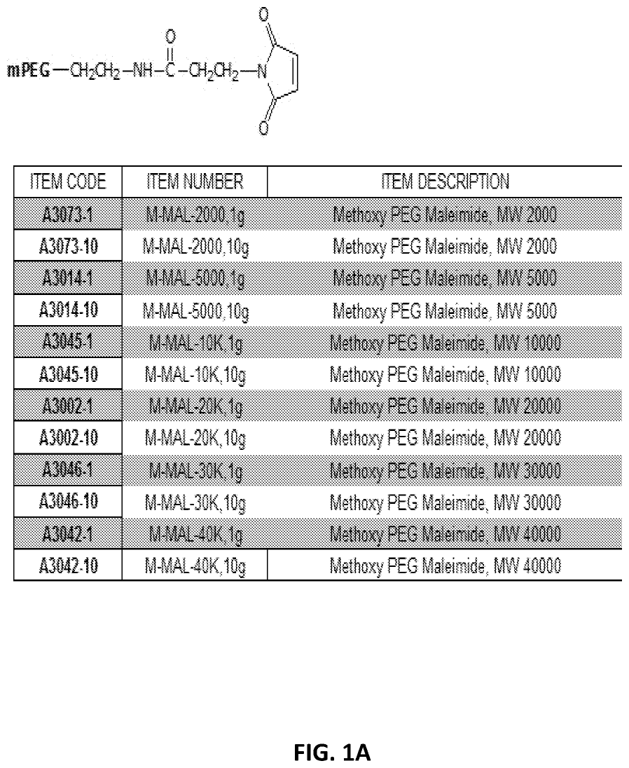

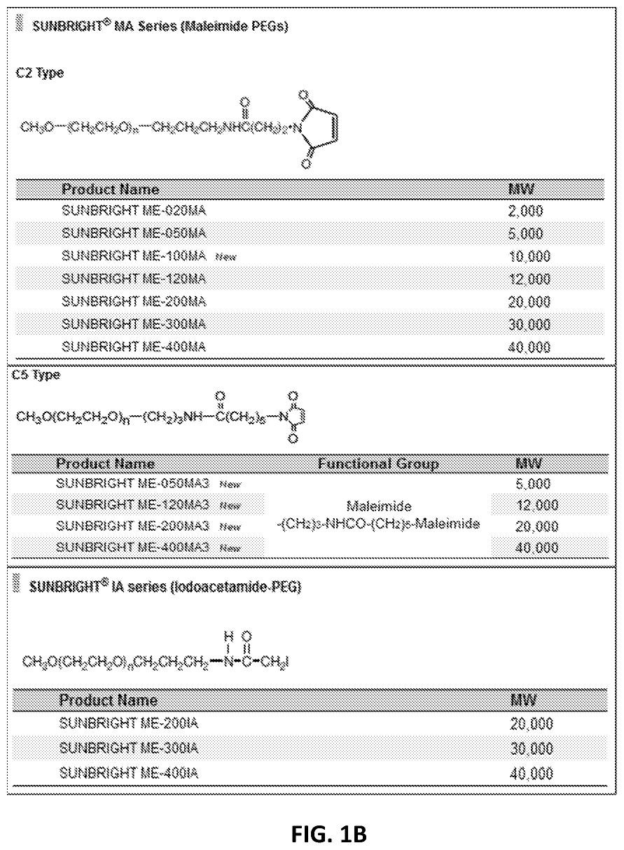

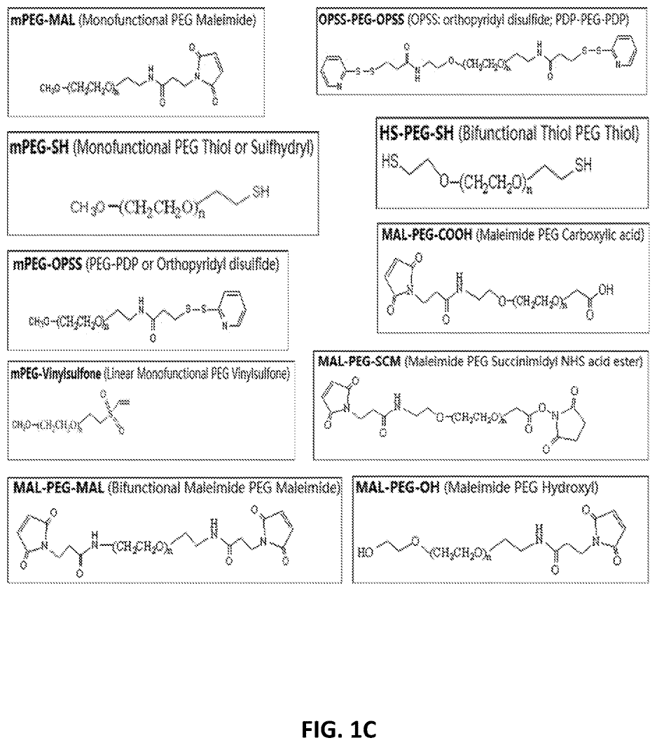

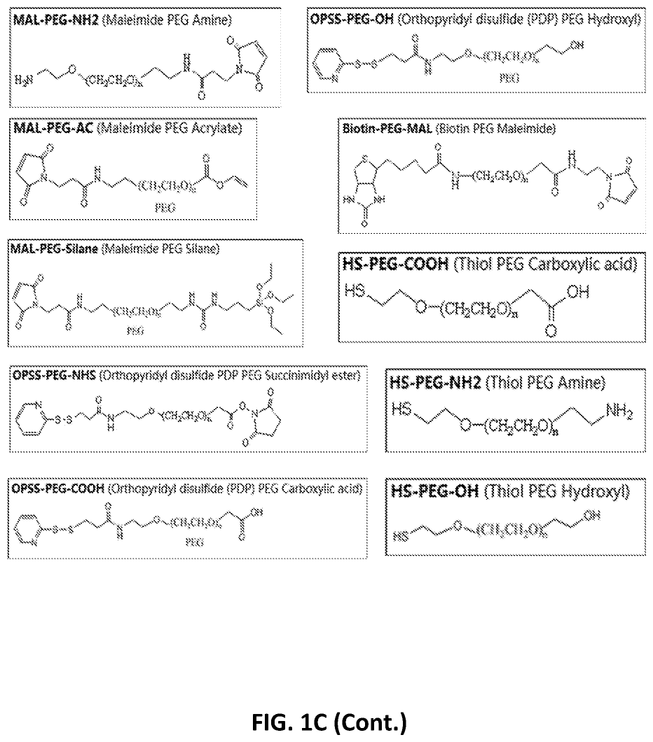

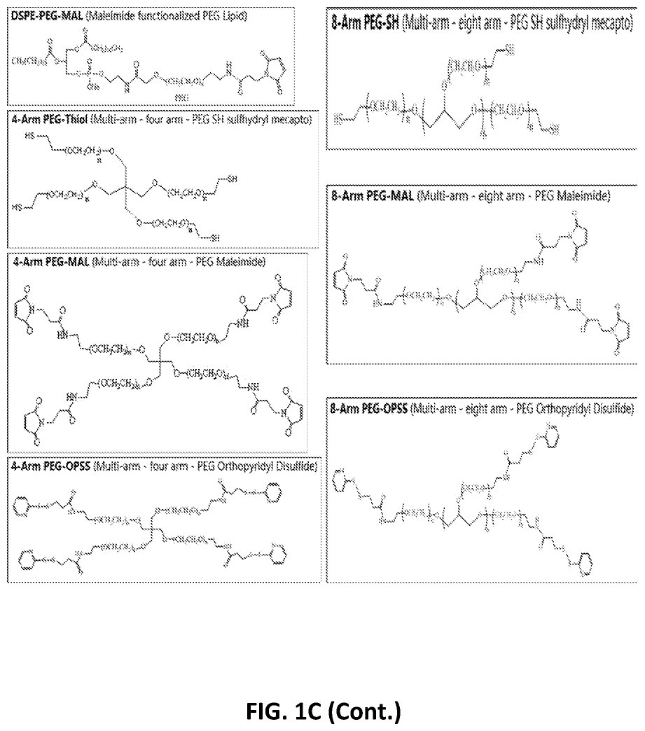

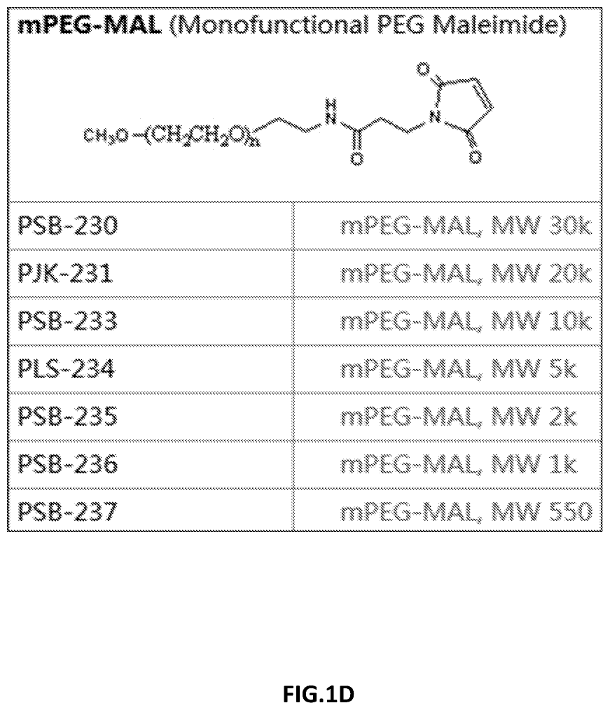

Non-limiting examples of thiol, sulfhydryl, or cysteine-reactive PEGs include Methoxy PEG Maleimides (M-PEG-MAL) (e.g., MW 2000, MW 5000, MW 10000, MW 20000, MW 30000, MW 40000). M-PEG-MALs react with the thiol groups on cysteine side chains in proteins and peptides to generate a stable 3-thiosuccinimidyl ether linkage. This reaction is highly selective and can take place under mild conditions at about pH 5.0-6.5 in the presence of other functional groups. Particular examples of commercially-available thiol, sulfyhdryl, or cysteine-reactive PEG molecules are illustrated in FIGS. 1A-1D. Thus, in certain embodiments, an ADI enzyme is conjugated to any one or more of the thiol, sulfhydryl, or cysteine-reactive PEG molecules described herein.

ADI may be covalently bonded to a modifying agent, such as PEG, with or without a linker, although a preferred embodiment utilizes a linker. ADI may be covalently bonded to PEG via a biocompatible linker using methods known in the art, as described, for example, by Park et al, Anticancer Res., 1:373-376 (1981); and Zaplipsky and Lee, Polyethylene Glycol Chemistry: Biotechnical and Biomedical Applications, J. M. Harris, ed., Plenum Press, NY, Chapter 21 (1992), the disclosures of which are hereby incorporated by reference herein in their entirety. In some instances, ADI may be coupled directly (i.e., without a linker) to a modifying agent such as PEG, for example, through an amino group, a sulfhydryl group, a hydroxyl group, a carboxyl group, or other group.

The linker used to covalently attach ADI to a modifying agent (e.g. PEG) can be any biocompatible linker. As discussed above, “biocompatible” indicates that the compound or group is non-toxic and may be utilized in vitro or in vivo without causing injury, sickness, disease, or death. A modifying agent such as PEG can be bonded to the linker, for example, via an ether bond, a thiol bond, an amide bond, or other bond.

In some embodiments, suitable linkers can have an overall chain length of about 1-100 atoms, 1-80 atoms, 1-60 atoms, 1-40 atoms, 1-30 atoms, 1-20 atoms, or 1, 2, 3, 4, 5, 6, 7, 8, 9, 10, 11, 12, 13, 14, 15, 16, 17, 18, 19, or 20 atoms, for example, wherein the atoms in the chain comprise C, S, N, P, and/or O. In certain embodiments, a linker is optional, e.g., a PEG conjugated ADI enzyme does not comprise a linker. In some instances, a linker group includes, for example, a succinyl group, an amide group, an imide group, a carbamate group, an ester group, an epoxy group, a carboxyl group, a hydroxyl group, a carbohydrate, a tyrosine group, a cysteine group, a histidine group, a methylene group, and combinations thereof. Particular examples of stable linkers include succinimide, propionic acid, carboxymethylate linkages, ethers, carbamates, amides, amines, carbamides, imides, aliphatic C—C bonds, and thio ethers. In certain embodiments, the biocompatible linker is a succinimidyl succinate (SS) group.

Other suitable linkers include an oxycarbonylimidazole group (including, for example, carbonylimidazole (CDI)), a nitro phenyl group (including, for example, nitrophenyl carbonate (NCP) or trichlorophenyl carbonate (TCP)), a trysylate group, an aldehyde group, an isocyanate group, a vinylsulfone group, or a primary amine. In certain embodiments, the linker is derived from SS, SPA, SCM, or NHS; in certain embodiments, SS, SPA, or NHS are used, and in some embodiments, SS or SPA are used. Thus, in certain embodiments, potential linkers can be formed from methoxy-PEG succinimidyl succinate (SS), methoxy-PEG succinimidyl glutarate (SG), methoxy-PEG succinimidyl carbonate (SC), methoxy-PEG succinimidyl carboxymethyl ester (SCM), methoxy-PEG2 N-hydroxy succinimide (NHS), methoxy-PEG succinimidyl butanoate (SBA), methoxy-PEG succinimidyl propionate (SPA), methoxy-PEG succinimidyl glutaramide, and/or methoxy-PEG succinimidyl succinimide.

Additional examples of linkers include, but are not limited to, one or more of the following: —O—, —NH—, —S—, —C(O)—, C(O)—NH, NH—C(O)—NH, O—C(O)—NH, —C(S)—, —CH2-, -CH2-CH2-, -CH2-CH2-CH2-, -CH2-CH2-CH2-CH2-, —O—CH2-, -CH2-O—, —O—CH2-CH2-, -CH2-O—CH2-, -CH2-CH2-O—, —O—CH2-CH2-CH2-, -CH2-O—CH2-CH2-, -CH2-CH2-O—CH2-, -CH2-CH2-CH2-O—, —O—CH2-CH2-CH2-CH2-, -CH2-O—CH2-CH2-CH2-, -CH2-CH2-O—CH2-CH2-, -CH2-CH2-CH2-O—CH2-, -CH2-CH2-CH2-CH2-O—, —C(O)—NH—CH2-, —C(O)—NH—CH2-CH2-, -CH2-C(O)—NH—CH2-, -CH2-CH2-C(O)—NH—, —C(O)—NH—CH2-CH2-CH2-, -CH2-C(O)—NH—CH2-CH2-, -CH2-CH2-C(O)—NH—CH2-, -CH2-CH2-CH2-C(O)—NH—, —C(O)—NH—CH2-CH2-CH2-CH2-, -CH2-C(O)—NH—CH2-CH2-CH2-, -CH2-CH2-C(O)—NH—CH2-CH2-, -CH2-CH2-CH2-C(O)—NH—CH2-, -CH2-CH2-CH2-C(O)—NH—CH2-CH2-, -CH2-CH2-CH2-CH2-C(O)—NH—, —NH—C(O)—CH2-, -CH2-NH—C(O)—CH2-, -CH2-CH2-NH—C(O)—CH2-, —NH—C(O)—CH2-CH2-, -CH2-NH—C(O)—CH2-CH2, —CH2-CH2-NH—C(O)—CH2-CH2, —C(O)—NH—CH2-, —C(O)—NH—CH2-CH2-, —O—C(O)—NH—CH2-, —O—C(O)—NH—CH2-CH2-, —NH—CH2-, —NH—CH2-CH2-, -CH2-NH—CH2-, -CH2-CH2-NH—CH2-, —C(O)—CH2-, —C(O)—CH2-CH2-, -CH2-C(O)—CH2-, -CH2-CH2-C(O)—CH2-, —CH2-CH2-C(O)—CH2-CH2-, -CH2-CH2-C(O)—, —CH2-CH2-CH2-C(O)—NH—CH2-CH2-NH—, —CH2-CH2-CH2-C(O)—NH—CH2-CH2-NH—C(O)—, —CH2-CH2-CH2-C(O)—NH—CH2-CH2-NH—C(O)—CH2-, bivalent cycloalkyl group, —N(R6)-, R6 is H or an organic radical selected from the group consisting of alkyl, substituted alkyl, alkenyl, substituted alkenyl, alkynyl, substituted alkynyl, aryl and substituted aryl. Additionally, any of the linker moieties described herein may further include an ethylene oxide oligomer chain comprising 1 to 20 ethylene oxide monomer units [i.e., —(CH₂CH₂O)₁₂₀—]. That is, the ethylene oxide oligomer chain can occur before or after the linker, and optionally in between any two atoms of a linker moiety comprised of two or more atoms. Also, the oligomer chain would not be considered part of the linker moiety if the oligomer is adjacent to a polymer segment and merely represent an extension of the polymer segment.

In certain embodiments, the ADI enzyme comprises one or more PEG molecules and/or linkers as described herein. In certain embodiments, the linker is a water-labile linker.

The attachment of PEG to ADI increases the circulating half-life of ADI. Generally, PEG is attached to a primary amine of ADI. Selection of the attachment site of PEG, or other modifying agent, on the ADI is determined by the role of each of the sites within the active domain of the protein, as would be known to the skilled artisan. PEG may be attached to the primary amines of ADI without substantial loss of enzymatic activity. For example, the lysine residues present in ADI are all possible points at which ADI as described herein can be attached to PEG via a biocompatible linker, such as SS, SPA, SCM, SSA and/or NHS. PEG may also be attached to other sites on ADI, as would be apparent to one skilled in the art in view of the present disclosure.

From 1 to about 30 PEG molecules may be covalently bonded to ADI. In certain embodiments, ADI is modified with (i.e., comprises) one PEG molecule. In some embodiments, the ADI is modified with more than one PEG molecule. In particular embodiments, the ADI is modified with about 1 to about 10, or from about 7 to about 15 PEG molecules, or from about 2 to about 8 or about 9 to about 12 PEG molecules. In some embodiments, the ADI is modified with about 2, 3, 4, 5, 6, 7, 8, 9, 10, 11, 12, 13, 14, or 15 PEG molecules. In specific embodiments, ADI is modified with 4.5-5.5 PEG molecules per ADI. In some embodiment, ADI is modified with 5±1.5 PEG molecules.

In certain embodiments, about 15% to about 70% of the primary amino groups in ADI are modified with PEG, in some embodiments about 20% to about 65%, about 25% to about 60%, or in certain embodiments about 30% to about 55%, or 45% to about 50%, or in some embodiments about 50% of the primary amino groups in arginine deiminase are modified with PEG. When PEG is covalently bonded to the end terminus of ADI, it may be desirable to have only 1 PEG molecule utilized. Increasing the number of PEG units on ADI increases the circulating half-life of the enzyme. However, increasing the number of PEG units on ADI decreases the specific activity of the enzyme. Thus, a balance needs to be achieved between the two, as would be apparent to one skilled in the art in view of the present disclosure.

In some embodiments, a common feature of biocompatible linkers is that they attach to a primary amine of arginine deiminase via a succinimide group. Once coupled with ADI, SS-PEG has an ester linkage next to the PEG, which may render this site sensitive to serum esterase, which may release PEG from ADI in the body. SPA-PEG and PEG2-NHS do not have an ester linkage, so they are not sensitive to serum esterase.

PEG which is attached to the protein may be either a straight chain, as with SS-PEG, SPA-PEG and SC-PEG, or a branched chain of PEG may be used, as with PEG2-NHS.

In some embodiments, for example, the amino acid substitutions employ non-natural amino acids for conjugation to PEG or other modifying agent (see, e.g., de Graaf et al., Bioconjug Chem. 20:1281-95, 2009). Certain embodiments thus include an ADI enzyme that is conjugated to one or more PEGs via one or more non-natural amino acids. In some embodiments the non-natural amino acid comprises a side chain having a functional group selected from the group consisting of: an alkyl, aryl, aryl halide, vinyl halide, alkyl halide, acetyl, ketone, aziridine, nitrile, nitro, halide, acyl, keto, azido, hydroxyl, hydrazine, cyano, halo, hydrazide, alkenyl, alkynyl, ether, thio ether, epoxide, sulfone, boronic acid, boronate ester, borane, phenylboronic acid, thiol, seleno, sulfonyl, borate, boronate, phospho, phosphono, phosphine, heterocyclic-, pyridyl, naphthyl, benzophenone, a constrained ring such as a cyclooctyne, thioester, enone, imine, aldehyde, ester, thioacid, hydroxylamine, amino, carboxylic acid, alpha-keto carboxylic acid, alpha or beta unsaturated acids and amides, glyoxyl amide, and an organosilane group. In some embodiments, the non-natural amino acid is selected from the group consisting of: p-acetyl-L-phenylalanine, O-methyl-L-tyrosine, L-3-(2-naphthyl)alanine, 3-methyl-phenylalanine, O-4-allyl-L-tyrosine, homocysteine, 4-propyl-L-tyrosine, tri-O-acetyl-GlcNAcp-serine, β-O-GlcNAc-L-serine, tri-O-acetyl-GalNAc-α-threonine, α-GalNAc-L-threonine, L-Dopa, a fluorinated phenylalanine, isopropyl-L-phenylalanine, p-azido-L-phenylalanine, p-acyl-L-phenylalanine, p-benzoyl-L-phenylalanine, L-phosphoserine, phosphonoserine, phosphonotyrosine, p-iodo-phenylalanine, p-bromophenylalanine, p-amino-L-phenylalanine, and isopropyl-L-phenylalanine.

While ADI-PEG is the illustrative modified ADI described herein, as would be recognized by the skilled person ADI may be modified with other polymers or appropriate molecules for the desired effect, in particular reducing antigenicity and increasing serum half-life.

It is to be understood that some embodiments are based on the understanding that certain structural characteristics of arginine deiminase may prevent or interfere with the proper and rapid renaturation when produced via recombinant technology. In particular, these structural characteristics hinder or prevent the enzyme from assuming an active conformation during recombinant production. In some embodiments, the term “active conformation” is defined as a three-dimensional structure that allows for enzymatic activity by unmodified or modified arginine deiminase. The active conformation may, in particular, be necessary for catalyzing the conversion of arginine into citrulline. The term “structural characteristic” may be defined as any trait, quality or property of the polypeptide chain resulting from a particular amino acid or combination of amino acids. For instance, arginine deiminase may contain an amino acid that results in a bend or kink in the normal peptide chain and thus hinders the enzyme from assuming an active conformation during renaturation of the enzyme. In particular, arginine deiminase from Mycoplasma hominis has a proline at the 210 position that may result in a bend or kink in the peptide chain, making it more difficult to renature the enzyme during recombinant production. It is to be understood that arginine deiminase derived from other organisms may also have sites corresponding to the 210 position of arginine deiminase from Mycoplasma hominis.

Some embodiments provide for specific amino acid substitutions in the polypeptide chain of wild type arginine deiminases. For instance, in some embodiments, the proline at position 210 (or the equivalent residue) is substituted with serine. Non-limiting examples of other substitutions include Pro210 to Thr210, Pro210 to Arg210, Pro210 to Asn210, Pro210 to Gln210 or Pro210 to Met210.

In specific embodiments, the modified ADI is ADI-PEG 20. ADI-PEG 20 refers to the ADI molecule described, for example, in U.S. Pat. Nos. 6,183,738; 6,635,462; Ascierto P A, et al. (2005) Pegylated arginine deiminase treatment of patients with metastatic melanoma: results from phase I and II studies. J Clin Oncol 23(30): 7660-7668; Izzo F, et al. (2004) Pegylated arginine deiminase treatment of patients with unresectable hepatocellular carcinoma: results from phase I/II studies. J Clin Oncol 22(10): 1815-1822; Holtsberg F W, et al. (2002), Poly(ethylene glycol) (PEG) conjugated arginine deiminase: effects of PEG formulations on its pharmacological properties. J Control Release 80(1-3): 259-271; and Kelly et al., (2012) British Journal of Cancer 106, 324-332. As would be recognized by the skilled artisan, ADI-PEG 20 is a pegylated ADI enzyme derived from M. hominis (mass average of about 5.5±1.0 bonded PEG 20,000 molecules), which has two substitutions (K112E; P210S) relative to the wild type M. hominis ADI sequence.

In certain embodiments, the ADI-PEG in the formulation retains at least 50, 55, 60, 65, 70, 75, 80, 85, 90, or 95% of its ADI activity, for example, relative to a corresponding ADI-PEG in a non-lyophilized liquid control composition. In some embodiments, the ADI-PEG retains at least 50, 55, 60, 65, 70, 75, 80, 85, 90, or 95% of its (original) ADI activity after being stored as a lyophilized formulation for about or at least about 1, 2, 3, or 4 weeks, or about or at least about 1, 2, 3, 4, 5, 6, 7, 8, 9, 10, 11, 12, 13, 14, 15, 16, 17, 18, 19, 20, 21, 22, 23, 24, 36, 48, 60, or 72 months. In some embodiments, the ADI activity is retained after being stored at a temperature of about 2-8° C., or at about room temperature, or at a stressed-temperature, for example, a temperature of about or at least about 50-60° C. In certain embodiments, the ADI-PEG retains at least 50, 55, 60, 65, 70, 75, 80, 85, 90, 95, or 100% of the (original) PEG molecules (per ADI monomer/protomer), for example, relative to a corresponding ADI-PEG in a non-lyophilized liquid control composition.

The skilled artisan will appreciate that the various ADI-PEG polypeptides described herein can be combined with any one or more of the various formulation components described herein, and used according to any one or more of the methods and compositions described herein.

### Reconstituted Liquid Compositions and Related Methods

Certain embodiments include reconstituted liquid compositions, methods of reconstituting the liquid compositions, and methods of using the same for arginine depletion therapies, including the treatment of various cancers.

For instance, certain embodiments include methods of reconstituting a lyophilized formulation described herein, comprising adding a pharmaceutically-acceptable solvent or diluent to the lyophilized formulation to form a reconstituted liquid composition. Also included are reconstituted liquid compositions prepared by any method described herein. Certain exemplary embodiments relate to reconstituted liquid compositions, comprising a lyophilized formulation described herein and a pharmaceutically-acceptable diluent or solvent, that is, where the lyophilized formulation is dissolved or otherwise solubilized in the solvent to form a liquid composition.

In particular embodiments, the solvent or diluent is water, or sterile water. In some instances, the lyophilized formulation is reconstituted to a substantially aggregate-free solution of about 5-20 mg/ml ADI-PEG (for example, about 5, 6, 7, 8, 9, 10, 11, 12, 13, 14, 15, 16, 17, 18, 19, or 20 mg/ml ADI-PEG) in a time of about or less than about five, four, three, two, or one minutes.

In some embodiments, the ADI-PEG in the reconstituted liquid composition retains at least 50, 55, 60, 65, 70, 75, 80, 85, 90, or 95% of its arginine deiminase (ADI) activity, for example, relative to a corresponding ADI-PEG in a non-lyophilized liquid control composition. In some embodiments, the ADI-PEG in the reconstituted liquid composition retains at least 50, 55, 60, 65, 70, 75, 80, 85, 90, or 95% of its ADI activity after being stored as a lyophilized formulation for about or at least about 1, 2, 3, or 4 weeks, or about or at least about 1, 2, 3, 4, 5, 6, 7, 8, 9, 10, 11, 12, 13, 14, 15, 16, 17, 18, 19, 20, 21, 22, 23, 24, 36, 48, 60, or 72 months. In some embodiments, the ADI activity is retained after being stored at a temperature of about 2-8° C., or at about room temperature, or at a stressed-temperature, for example, a temperature of about or at least about 50-60° C. In some embodiments, the specific ADI enzyme activity of the ADI-PEG in the reconstituted liquid formulation is about 5.0 to about 120 IU/mg, or about 5.5, 6, 6.5, 7, 7.5, 8, 8.5, 9.0, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 35, 40, 45, 50, 55, 55, 60, 65, 70, 75, 80, 85, 90, 95, 100, 105, 110, 115, or 120 IU/mg, including all integers and ranges in between.

In certain embodiments, ADI-PEG in the reconstituted liquid composition retains at least 50, 55, 60, 65, 70, 75, 80, 85, 90, 95, or 100% of the (original) PEG molecules (per ADI monomer/protomer) upon reconstitution, for example, relative to a corresponding ADI-PEG in a non-lyophilized liquid control composition. In some embodiments, ADI-PEG in the reconstituted liquid composition retains at least 50, 55, 60, 65, 70, 75, 80, 85, 90, 95, or 100% of the (original) PEG molecules (per ADI monomer/protomer) upon reconstitution, for example, after being stored as a lyophilized formulation for about or at least about 1, 2, 3, or 4 weeks, or about or at least about 1, 2, 3, 4, 5, 6, 7, 8, 9, 10, 11, 12, 13, 14, 15, 16, 17, 18, 19, 20, 21, 22, 23, 24, 36, 48, 60, or 72 months, optionally after being stored at a temperature of about 2-8° C. and/or about room temperature.

In some embodiments, the ADI-PEG in the liquid composition has an osmolality of about 50 mOsm/kg to about 500 mOsm/kg, or about 50, 55, 60, 65, 70, 75, 80, 85, 90, 95, 100, 105, 110, 120, 125, 130, 135, 140, 145, 150, 155, 160, 165, 170, 175, 180, 185, 190, 195, 200, 205, 210, 220, 225, 230, 235, 240, 245, 250, 255, 260, 265, 270, 275, 280, 285, 290, 295, 300, 305, 310, 320, 325, 330, 335, 340, 345, 350, 355, 360, 365, 370, 375, 380, 385, 390, 395, 400, 405, 410, 420, 425, 430, 435, 440, 445, 450, 455, 460, 465, 470, 475, 480, 485, 490, 495, or about 500 mOsm/kg, including all integers and ranges in between. In some embodiments, the reconstituted liquid composition is suitable for injection into a subject, for example, a human subject.

Also included are methods of using the reconstituted liquid compositions for arginine depletion therapies. For example, certain embodiments include methods of treating, ameliorating the symptoms of, or inhibiting the progression of, a cancer in a subject in need thereof, comprising administering to the subject a reconstituted liquid formulation described herein.

The methods and compositions described herein can be used in the treatment of any variety of cancers. In some embodiments, the cancer is selected from one or more of hepatocellular carcinoma (HCC), melanoma, metastatic melanoma, pancreatic cancer, prostate cancer, small cell lung cancer, mesothelioma, lymphocytic leukemia, chronic myelogenous leukemia, lymphoma, hepatoma, sarcoma, leukemia, acute myeloid leukemia, relapsed acute myeloid leukemia, B-cell malignancy, breast cancer, ovarian cancer, colorectal cancer, gastric cancer, glioma (e.g., astrocytoma, oligodendroglioma, ependymoma, or a choroid plexus papilloma), glioblastoma multiforme (e.g., giant cell gliobastoma or a gliosarcoma), meningioma, pituitary adenoma, vestibular schwannoma, primary CNS lymphoma, primitive neuroectodermal tumor (medulloblastoma), non-small cell lung cancer (NSCLC), kidney cancer, bladder cancer, uterine cancer, esophageal cancer, brain cancer, head and neck cancers, cervical cancer, testicular cancer, and stomach cancer.

In some embodiments, the cancer exhibits reduced expression and/or activity of argininosuccinate synthetase-1 (ASS-1), or is otherwise argininosuccinate synthetase-1-deficient. In some instances, reduced ASS-1 expression or activity is a reduction in expression and/or activity of about 5%, 10%, 15%, 20%, 25%, 30%, 35%, 40%, 45%, 50%, 55%, 60%, 65%, 70%, or more, relative to expression and/or activity in an appropriate control sample, for example, a normal cell or tissue. In certain embodiments, ASS or ASL expression or activity is reduced by at least two-fold relative to expression or activity in a control sample. Reduction in ASS-1 expression or activity can be measured according to routine techniques the art, including, for example, quantitative PCR, immunohistochemistry, enzyme activity assays (e.g., ADI activity assays to measure conversion of citrulline into argininosuccinate or conversion of argininosuccinate into arginine and fumarate), and the like.

In some embodiments, the methods or compositions described herein increase median survival time of a patient by 4 weeks, 5 weeks, 6 weeks, 7 weeks, 8 weeks, 9 weeks, 10 weeks, 15 weeks, 20 weeks, 25 weeks, 30 weeks, 40 weeks, or longer. In certain embodiments, the methods or compositions described herein increase median survival time of a patient by 1 year, 2 years, 3 years, or longer. In some embodiments, the methods or compositions described herein increase progression-free survival by 2 weeks, 3 weeks, 4 weeks, 5 weeks, 6 weeks, 7 weeks, 8 weeks, 9 weeks, 10 weeks or longer. In certain embodiments, the methods or compositions described herein increase progression-free survival by 1 year, 2 years, 3 years, or longer.

In certain embodiments, the composition administered is sufficient to result in tumor regression, as indicated by a statistically significant decrease in the amount of viable tumor, for example, at least a 10%, 20%, 30%, 40%, 50% or greater decrease in tumor mass, or by altered (e.g., decreased with statistical significance) scan dimensions. In certain embodiments, the composition administered is sufficient to result in stable disease. In certain embodiments, the composition administered is sufficient to result in stabilization or clinically relevant reduction in symptoms of a particular disease indication known to the skilled clinician.

The methods or compositions for treating cancers can be combined with other therapeutic modalities. For example, a compositions described herein can be administered to a subject before, during, or after other therapeutic interventions, including symptomatic care, radiotherapy, surgery, transplantation, hormone therapy, photodynamic therapy, antibiotic therapy, or any combination thereof. Symptomatic care includes administration of corticosteroids, to reduce cerebral edema, headaches, cognitive dysfunction, and emesis, and administration of anti-convulsants, to reduce seizures. Radiotherapy includes whole-brain irradiation, fractionated radiotherapy, and radiosurgery, such as stereotactic radiosurgery, which can be further combined with traditional surgery.

Methods for identifying subjects with one or more of the diseases or conditions described herein are known in the art.

Administration may be achieved by a variety of different routes, including oral, parenteral, intranasal, intravenous, intradermal, intramuscular, intrathecal, subcutaneous, sublingual, buccal, rectal, vaginal, and topical. Preferred modes of administration depend upon the nature of the condition to be treated or prevented. Particular embodiments include administration by IV infusion.

The precise dosage and duration of treatment is a function of the disease being treated and may be determined empirically using known testing protocols or by testing the compositions in model systems known in the art and extrapolating therefrom. Controlled clinical trials may also be performed. Dosages may also vary with the severity of the condition to be alleviated. A pharmaceutical composition is generally formulated and administered to exert a therapeutically useful effect while minimizing undesirable side effects. The composition may be administered one time, or may be divided into a number of smaller doses to be administered at intervals of time. For any particular subject, specific dosage regimens may be adjusted over time according to the individual need.

In some embodiments, a therapeutically effective amount or therapeutic dosage of a composition described herein is an amount that is effective to reduce or stabilize tumor growth. In certain instances, treatment is initiated with small dosages which can be increased by small increments until the optimum effect under the circumstances is achieved.

In some embodiments, a dosage is administered from about once a day to about once every two or three weeks. For example, in certain embodiments, a dosage is administered about once every 1, 2, 3, 4, 5, 6, or 7 days, or about once a week, or about twice a week, or about three times a week, or about once every two or three weeks.

In some embodiments, the dosage is from about 0.1 mg/kg to about 20 mg/kg, or to about 10 mg/kg, or to about 5 mg/kg, or to about 3 mg/kg. In some embodiments, the dosage is about 0.10 mg/kg, 0.15 mg/kg, 0.20 mg/kg, 0.25 mg/kg, 0.30 mg/kg, 0.35 mg/kg, 0.40 mg/kg, 0.45 mg/kg, 0.50 mg/kg, 0.55 mg/kg, 0.60 mg/kg, 0.65 mg/kg, 0.70 mg/kg, 0.75 mg/kg, 0.80 mg/kg, 0.85 mg/kg, 0.90 mg/kg, 0.95 mg/kg, 1.0 mg/kg, 1.5 mg/kg, 2.0 mg/kg, 2.5 mg/kg, 3.0 mg/kg, 3.5 mg/kg, 4.0 mg/kg, 4.5 mg/kg, 5.0 mg/kg, 5.5 mg/kg, 6.0 mg/kg, 6.5 mg/kg. 7.0 mg/kg, 7.5 mg/kg, 8.0 mg/kg, 8.5 mg/kg, 9.0 mg/kg, 9.5 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, or 20 mg/kg, including all integers and ranges in between. In specific embodiments, the dosage is about 1 mg/kg once a week as a 2 ml intravenous injection to about 20 mg/kg once every 3 days.

In some embodiments, the dosage is from about 50 IU/m² to about 1000 IU/m². In particular embodiments, the dosage is about 50 IU/m², 60 IU/m², 70 IU/m², 80 IU/m², 90 IU/m², 100 IU/m², 110 IU/m², 120 IU/m², 130 IU/m², 140 IU/m², 150 IU/m², 160 IU/m², 170 IU/m², 180 IU/m², 190 IU/m², 200 IU/m², 210 IU/m², 220 IU/m², 230 IU/m², 240 IU/m², 250 IU/m², 260 IU/m², 270 IU/m², 280 IU/m², 290 IU/m², 300 IU/m², 310 IU/m², about 320 IU/m², about 330 IU/m², 340 IU/m² about 350 IU/m², 360 IU/m², 370 IU/m², 380 IU/m², 390 IU/m², 400 IU/m², 410 IU/m², 420 IU/m², 430 IU/m², 440 IU/m², 450 IU/m², 500 IU/m², 550 IU/m², 600 IU/m², 620 IU/m², 630 IU/m², 640 IU/m², 650 IU/m², 660 IU/m², 670 IU/m², 680 IU/m², 690 IU/m², 700 IU/m², 710 IU/m², 720 IU/m², 730 IU/m², 740 IU/m², 750 IU/m², 760 IU/m², 770 IU/m², 780 IU/m², 790 IU/m², 800 IU/m², 810 IU/m², 820 IU/m², 830 IU/m², 840 IU/m², 850 IU/m², 860 IU/m², 870 IU/m², 880 IU/m², 890 IU/m², 900 IU/m², 910 IU/m², 920 IU/m², 930 IU/m², 940 IU/m², 950 IU/m², 960 IU/m², 970 IU/m², 980 IU/m², 990 IU/m², or about 1000 IU/m², including all integers and ranges in between.

Also included are patient care kits, comprising one or more lyophilized formulations described herein. Certain kits also comprise one or more pharmaceutically-acceptable diluents or solvents, such as water (e.g., sterile water). In some embodiments, the lyophilized formulations are stored in vials, cartridges, dual chamber syringes, and/or pre-filled mixing systems.

The kits herein may also include a one or more additional therapeutic agents or other components suitable or desired for the indication being treated, or for the desired diagnostic application. The kits herein can also include one or more syringes or other components necessary or desired to facilitate an intended mode of delivery (e.g., stents, implantable depots, etc.).

All publications, patent applications, and issued patents cited in this specification are herein incorporated by reference as if each individual publication, patent application, or issued patent were specifically and individually indicated to be incorporated by reference.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims. The following examples are provided by way of illustration only and not by way of limitation. Those of skill in the art will readily recognize a variety of noncritical parameters that could be changed or modified to yield essentially similar results.

### EXAMPLES

### Example 1

In this study, various excipient types were evaluated for their ability to provide a lyophilized ADI-PEG 20 formulation with elegant cake appearance and maintained stability after the lyophilization process and reconstitution. Table E1 below provides a summary of the formulations tested in this study.

TABLE E1

| Formu- | | | | |
|---|---|---|---|---|
| lation | | Cryopro- | Lyopro- | Bulking |
| ID | Composition | tectant | tectant | Agent |
| F1 | 20 mM Sodium phosphate | No | No | No |
| | 130 mM Sodium chloride | | | |
| F2 | 20 mM histidine | No | No | No |
| F3 | 20 mM histidine | Yes | Yes | No |
| | 5% Sucrose | | | |
| F4 | 20 mM histidine | No | No | Yes |
| | 5% Mannitol | | | |
| F5 | 20 mM histidine | Yes | No | Yes |
| | 1% Dextran 40 | | | |
| F6 | 20 mM histidine | Yes | Yes | Yes |
| | 1% Dextran 40 | | | |
| | 5% Sucrose | | | |
| F7 | 20 mM histidine | Yes | Yes | Yes |
| | 5% Trehalose | | | |
| F8 | 20 mM histidine | Yes | Yes | Yes |
| | 4% Mannitol | | | |
| | 1% Sucrose | | | |
| F9 | 20 mM histidine | Yes | Yes | No |
| | 4.8% Sucrose | | | |
| | 0.01% Tween-80* | | | |
| F10 | 20 mM histidine | Yes | Yes | No |
| | 4.8% Sucrose | | | |
| | 0.01% Tween-80* | | | |
| | 0.25% Glycerol** | | | |

Formulations at pH 6.8 ± 0.1
*Surfactant
**Plasticizer

The formulations of Table E1 were lyophilized in vials using a VirTis Genesis 25XL Lyophilizer. The shelves were precooled to 5° C. prior to loading the vials. The lyophilization cycle was as follows:
The shelf was maintained at 6 C for 30 minutes;
the shelf was cooled to −40° C. at 1° C./min and held at −40° C. for one hour;
the shelf was heated to −35° C. at 1° C./min where primary drying occurred at 50 mtorr for 72 hours;
the shelf was heated to 20° C. at 0.1° C./min where secondary drying occurred at 50 mtorr for 8 hours.

The lyophilized formulations were evaluated by visual appearance before and after stressed-storage conditions of 60° C. for one week. All lyophilized formulations showed a white coloring after lyophilization, and an off-white to yellow coloring after stressed storage conditions.

The lyophilized formulations were also evaluated for enzyme activity and by size-exclusion chromatography relative to pre-lyophilized formulations (unshaded results), and also before and after stressed-storage conditions (shaded results). The results are shown in Tables E2 and E3 below.

TABLE E2
Enzyme Activity Evaluation ADI-PEG 20 Activity in IU/mL.

| | | | % activity | | 1 wk at | % activity | |
|---|---|---|---|---|---|---|---|
| Formulation | Pre-lyo | Post-lyo | retained | Rank | 60° C. | retained | Rank |
| F1 | 163 | 107 | 66 | + | 6 | 6 | ++ |
| F2 | 161 | 132 | 82 | +++ | 1 | 1 | + |
| F3 | 169 | 137 | 81 | +++ | 13 | 9 | ++ |
| F4 | 155 | 128 | 83 | +++ | 0 | 0 | + |
| F5 | 154 | 126 | 82 | +++ | 10 | 8 | ++ |
| F6 | 132 | 131 | 99 | ++++ | 14 | 11 | +++ |
| F7 | 147 | 152 | 103 | ++++ | 30 | 20 | +++ |
| F8 | 132 | 138 | 105 | ++++ | 34 | 25 | ++++ |
| F9 | 135 | 118 | 87 | +++ | 9 | 8 | ++ |
| F10 | 150 | 137 | 91 | ++++ | 8 | 6 | ++ |

TABLE E3
ADI-PEG 20 Apparent Concentration (mg/mL) Evaluation by SEC.

| | | | % activity | | 1 wk at | % activity | |
|---|---|---|---|---|---|---|---|
| Formulation | Pre-lyo | Post-lyo | retained | Rank | 60° C. | retained | Rank |
| F1 | 11.0 | 10.3 | 94 | ++ | 0.5 | 5 | + |
| F2 | 13.9 | 12.7 | 91 | ++ | 7.9 | 62 | ++ |
| F3 | 14.1 | 12.4 | 88 | + | 13 | 105 | +++ |
| F4 | 14.2 | 12.6 | 89 | + | 3.4 | 27 | + |
| F5 | 14.3 | 13 | 91 | ++ | 14.1 | 108 | +++ |
| F6 | 14.3 | 12.9 | 90 | ++ | 13.7 | 106 | +++ |
| F7 | 14.4 | 12.9 | 90 | ++ | 13.8 | 107 | +++ |
| F8 | 13.7 | 12.2 | 89 | + | 13.3 | 109 | +++ |
| F9 | 13.4 | 12.2 | 91 | ++ | 13.2 | 108 | +++ |
| F10 | 13.7 | 12.6 | 92 | ++ | 10.2 | 81 | ++ |

Total and free PEG levels (mg/ml) was also evaluated (data not shown) and the formulations were ranked and grouped according to the total number of + values assigned). The rankings from highest (Group 1) to lowest (Group 5) are shown in Table E4 below.

TABLE E4

| | | | Cryo- | Lyo- | Bulking | | |
|---|---|---|---|---|---|---|---|
| Rank | ID | Composition | protectant | protectant | Agent | Surfactant | Plasticizer |
| Group 1 | F6, F7, | F6: H + Dextran | Yes | Yes | Yes | No | No |
| | and F8 | 40 + Sucrose | | | | | |
| | | F7: H + Trehalose | | | | | |
| | | F8: H + Mannitol + | | | | | |
| | | Sucrose | | | | | |
| Group 2 | F5 | H + Dextran 40 | Yes | No | Yes | No | No |
| Group 3 | F3, F9, | F3: H + Sucrose | Yes | Yes | Yes | No: F3 | No: F3 |
| | and F10 | F9: H + Sucrose + | | | | Yes: F9 | No: F9 |
| | | Tween 80 | | | | Yes: F10 | Yes: F10 |
| | | F10: H + Sucrose + | | | | | |
| | | Tween 80 + Glycerol | | | | | |
| Group 4 | F2 | Histidine (H) | No | No | No | No | No |
| Group 5 | F1 and | F1: Phosphate | No | No | No: F1 | No | No |
| | F4 | F4: H + Mannitol | | | Yes: F4 | | |

To summarize, all of the ADI-PEG 20 formulations (F1-F10) prepared in this study resulted in elegant, uniform lyophilized cakes, and all appeared to remain stable based on the assessment from enzymatic activity and size exclusion chromatography. Results from the stressed storage conditions (60° C. for one week) indicate that the best lyophilized ADI-PEG 20 formulations contain a cryoprotectant and a lyoprotectant. The addition of a bulking agent such as dextran 40 or crystalline mannitol, or a high glass transition temperature excipient such as trehalose, appeared to attenuate activity loss and improve formation of the PEG molecules after reconstitution.

### Example 2

In this study, formulation development included (a) buffer evaluation (histidine, citrate, and glycyl-glycine buffers, pH 6-7) and (b) combination evaluation (bulking agents trehalose and sucrose; and stabilizers proline, glycine; EDTA to enhance enzyme activity). The evaluation of formulations used a rapid lyophilization cycle (FAST LYO cycle; freezing followed by secondary drying).

Table E5 below provides a summary of the formulations tested for buffer evaluation. Also provided are certain pre-lyophilization characteristics. The pre-lyophilization liquid samples were evaluated after dialysis for RALS (Right Angle Light Scattering) and concentration, and also for RALS in combination with an acute temperature ramp.

TABLE E5

| | | % Recovery | | |
|---|---|---|---|---|
| | | based on A280 | | |
| Buffer | pH | Concentration | Appearance | RALS |
| 20 mM Phosphate, | 6.75 | N/A | Clear | 1.451 |
| 130 mM NaCl | | | | |
| 20 mM Histidine | 5.99 | 93 | Clear | 2.519 |
| 20 mM Histidine | 6.42 | 93 | Clear | 2.555 |
| 20 mM Histidine | 6.92 | 100 | Clear | 2.823 |
| 20 mM Sodium citrate | 6.15 | 89 | Clear | 4.534 |
| 20 mM Sodium citrate | 6.68 | 97 | Clear | 3.662 |
| 20 mM Glycyl-Glycine | 6.71 | 88 | Clear | 1.859 |
| 20 mM Glycyl-Glycine | 7.05 | 93 | Clear | 2.025 |

The results of percentage recovery were relatively high and all formulations remained clear after dialysis, indicating no buffer incompatibility issues. The values of RALS were higher for the citrate-containing formulations which could indicate soluble aggregates.

Lyophilization was then performed after dialysis using Formatch's FAST LYO cycle in an SP Durastop lyophilizer. A FAST LYO cycle was performed without a primary drying cycle; instead, samples were dried using only a secondary drying cycle. Lyophilization cycle used to evaluate the buffer selection was: freeze at −50° C. for 2 hours (2° C./min); dry at 22° C. for 4 hours at 60 mtorr (all ramping at 2° C./min).

All lyophilized buffer formulations from Table E5 were evaluated using RALS and IF (Intrinsic Fluorescence). Both techniques were used to evaluate the heat stability in different formulations. Samples were heated from 20° C. to 90° C. This temperature scan revealed the Tm, the temperature where a sharp change occurred, reflecting heat-induced denaturation or unfolding of the protein. The protein was considered more stable if higher Tm values were detected.

The transition temperature was observed to be ~60° C. for both RALS and IF for all liquid control formulations at T(0). The post-dialysis liquid formulations were separately filtered through a 0.2 μm filter prior to lyophilization. Also, there were no significant differences between the buffer formulations. The formulations containing histidine pH 6.5 and 7.0 showed slightly better performed by RALS analysis, and the formulation containing histidine buffer with a pH value of 6.5 showed the overall lowest initial RALS readings indicating the least amount of soluble aggregates.

For the combination evaluation, a base buffer of 20 mM histidine pH 6.5 was employed. Trehalose, sucrose, and combinations thereof were evaluated as bulking agents and for their ability to provide isotonicity to the formulations. The amino acids proline and glycine were evaluated as stabilizers. Additionally, EDTA was evaluated as a chelator. Table E6 below provides a summary of the formulations tested for combination evaluation.

TABLE E6

| Formu- | | | | |
|---|---|---|---|---|
| lation | | Bulking | | Osmolality |
| ID | Composition | Agent | Stabilizer | (mOsmol/kg) |
| F1 | 20 mM sodium | No | No | 300 |
| | phosphate | | | |
| | 130 mM NaCl | | | |
| | pH 6.8 | | | |
| F2 | 20 mM histidine | No | No | 39 |
| | pH 6.5 | | | |
| F3 | 20 mM histidine | 9.5% trehalose | No | 325 |
| | pH 6.5 | | | |
| F4 | 20 mM histidine | 9.5% trehalose | 20 mM | 332 |
| | pH 6.5 | | proline | |
| F5 | 20 mM histidine | 9.5% trehalose | 20 mM | 465 |
| | pH 6.5 | | glycine | |
| F6 | 20 mM histidine | 9.5% sucrose | No | 436 |
| | pH 6.5 | | | |
| F7 | 20 mM histidine | 4.5% sucrose + | No | 328 |
| | pH 6.5 | 4.5% trehalose | | |
| F8 | 20 mM histidine | 9.5% | 0.05% | 313 |
| | pH 6.5 | trehalose | EDTA | |

Lyophilization was performed after dialysis using Formatch's FAST LYO cycle in an SP Durastop lyophilizer. The lyophilization cycle used to evaluate the combination study is as follows: freeze at −50° C. for 2 hours; dry at 22 C overnight at 75 mtorr (all ramping at 1° C./min). ADI-PEG 20 samples were lyophilized at 11.1 mg/mL (1 mL fill in a 10 mL vial).

The RALS temperature ramping data, RALS initial values, and enzymatic activity results suggest that a formulation containing sucrose provides an optimal stabilizing lyophilizable formulation for ADI-PEG 20. A formulation containing trehalose or both trehalose and sucrose also provides a stabilizing formulation. Additional excipients (e.g., glycine, proline, EDTA) did not significantly enhance the activity of the lyophilized formulations.

Formulations containing 9% sucrose or 9% trehalose were also evaluated by modulated differential scanning calorimetry (MDSC) to determine glass transition. The results of this analysis (a eutectic point at about −16° C. to −18° C.) suggest a primary drying temperature of −20° C. or lower should result in a non-collapsed lyophilized cake. The lyophilization cycle in Table E7 was then evaluated and showed acceptable cake appearance.

TABLE E7

| | | | | Duration for |
|---|---|---|---|---|
| Step | Temperature | Pressure | Ramp rate | Drying |
| Freezing | −50° C. | Atmos. | 1.0° C./min | 2 hours |
| Primary Drying | −20° C. | 75 mtorr | 1.0° C./min | 22 hours + |
| | | | | Ramp time |
| Secondary Drying | +20° C. | 200 mtorr | 1.0° C./min | 7 hours + |
| | | | | Ramp time |

To summarize, the buffer evaluation (histidine, citrate and glycyl-glycine buffers shows that all buffers were acceptable but identified the histidine buffer (pH 6.5) as the best lyophilization buffer. The combination evaluation (bulking agents trehalose and sucrose; stabilizers proline, glycine; EDTA to enhance enzymatic activity) indicated that formulations containing sucrose showed the best overall results. However, the formulation containing trehalose and combined sucrose/trehalose also performed well. Enzyme activity results for all buffers showed greater than 90% recovery after lyophilization and greater than 80% recovery after accelerated stability (5 days incubation at 50° C.) compared to the corresponding liquid controls. Reconstitution time for all buffers was less than 1 minute and all cakes showed 100% purity by size-exclusion chromatography.

### Example 3

In this study, two different lyophilization cycles were evaluated with each of three different formulations of 11 mg/mL ADI-PEG 20 at a fill volume of 3.5 mL/vial. The evaluated lyophilization parameters were shelf temperature, chamber pressure, and freezing conditions during drying. Table E8 below provides a summary of the formulations tested in this study.

TABLE E8

| Formulation | | Bulking | | | |
|---|---|---|---|---|---|
| ID | Buffer | Agent | Stabilizer | pH | Surfactant |
| 1 | 10 mM | 1.9% Glycine | 1% | 6.8 | 0.01% P20 |
| | histidine | | Trehalose | | |
| 2 | 10 mM | 8.5% sucrose | 1% | 6.8 | No |
| | histidine | | Trehalose | | |
| 3 | 10 mM | 9.0% sucrose | No | 6.8 | No |
| | histidine | | | | |

As noted above, two different lyophilization cycles were tested: an aggressive cycle (Lyophilization Cycle 1) and an intermediate cycle (Lyophilization Cycle 2).

The parameters for Lyophilization Cycle 1 are summarized in Table E9 below.

TABLE E9
Lyophilization Cycle 1.

| | | | | | Chamber |
|---|---|---|---|---|---|
| Step | Temperature | Time | | Ramp rate | pressure |
| Loading | 5° C. | N/A | | N/A | N/A |
| Freezing | 5° C. to −50° C. | N/A | | 1° C./min | N/A |
| | −50° C. | 2 | hours | N/A | N/A |
| Annealing | −50° C. to −20° C. | N/A | | 1° C./min | N/A |
| | −20° C. | 2 | hours | N/A | N/A |
| Primary | −20° C. | 1 | hour | N/A | 100 mTorr |
| drying | −20° C. to 20° C. | N/A | | 1° C./min | 100 mTorr |
| | 20° C. | 22.5 | hours | N/A | 100 mTorr |
| Secondary | 20° C. to 30° C. | N/A | | 1° C./min | 100 mTorr |
| drying | 30° C. | 10 | hours | N/A | 100 mTorr |

Upon completing Cycle 1, the resulting lyophiles were analyzed. After reconstitution with 3.5 mL of water Formulation ID 1 was visibly cloudy, whereas Formulation IDs 2 and 3 were clear and without particles.

Fourier Transform Infrared Spectroscopy (FTIR), differential scanning calorimetry (DSC), and SDS-PAGE analyses were performed on the lyophilized and reconstituted products from Cycle 1. Some structure changes in β-sheet (1630 cm-1) were observed in Formulation IDs 2 and 3 upon drying, but the structure of ADI-PEG 20 returned to its original state for these two formulations following reconstitution. Major structural changes were detected for Formulation ID 1 upon drying. After reconstitution, the structure of ADI-PEG 20 in this formulation appeared similar, but not identical to the pre-lyophilization sample. SDS-PAGE analysis showed no degradation peaks in any of the formulations.

To moderate the rate of sublimation between conservative and aggressive cycles, a shelf temperature of 0° C. was designed for primary drying in Lyophilization Cycle 2. The parameters for the intermediate Lyophilization Cycle 2 are summarized in Table E10 below.

TABLE E10
Lyophilization Cycle 2.

| | | | | | Chamber |
|---|---|---|---|---|---|
| Step | Temperature | Time | | Ramp rate | pressure |
| Loading | 5° C. | N/A | | N/A | N/A |
| Freezing | 5° C. to −50° C. | N/A | | 1° C./min | N/A |
| | −50° C. | 2 | hours | N/A | N/A |
| Annealing | −50° C. to −20° C. | N/A | | 1° C./min | N/A |
| | −20° C. | 2 | hours | N/A | N/A |
| Primary | −20° C. | 0.5 | hour | N/A | 100 mTorr |
| drying | −20° C. to 0° C. | N/A | | 1° C./min | 100 mTorr |
| | 0° C. | 17.5 | hours | N/A | 100 mTorr |
| Secondary | 0° C. to 25° C. | N/A | | 1° C./min | 50 mTorr |
| drying | 25° C. | 10 | hours | N/A | 50 mTorr |

Upon completing Cycle 2, the resulting lyophiles were analyzed. After reconstitution with 3.5 mL of water Formulation ID 1 was visibly turbid, while Formulation IDs 2 and 3 were clear and colorless.

FTIR, DSC, and SDS-PAGE analyses were performed on the lyophilized and reconstituted products from Lyophilization Cycle 2. Some structure changes in β-sheet (1630 cm-1) were observed in Formulation IDs 2 and 3 upon drying, but the structure of ADI-PEG 20 returned to its original state for these two formulations following reconstitution. Significant changes in structure were also detected for Formulation ID 1 upon drying. After reconstitution, the structure of ADI-PEG 20 in this formulation was similar to the structure prior to lyophilization. SDS-PAGE analysis showed no degradation peaks in any of the formulations.

Lyophilization Cycle 2 was performed again to confirm its robustness as an optimized drying cycle. Only the Formulations 2 and 3 were dried in this follow-up lyophilization cycle. Reconstituted samples of Formulation IDs 2 and 3 were examined by SDS-PAGE for physical degradation. No degradation products were observed for the reconstituted samples when the results were compared to the liquid drug substance and the pre-lyophilization samples.

To summarize, this study evaluated aggressive and intermediate lyophilization cycles with three the different formulations of ADI-PEG 20. Following each cycle, samples were analyzed for cake appearance, moisture content, turbidity, changes in secondary structure, physical stability and via DSC. Formulation ID 1 (w/glycine) showed signs of precipitation upon reconstitution following cycles 1 and 2, and was excluded from the confirmatory lyophilization cycle. Conversely, the integrity of the Formulation IDs 2 and 3 containing sucrose was well maintained following each tested lyophilization cycle. Any changes in the structure after drying were reversible upon reconstitution.

### Example 4

In this study, two different PEG-numbered ADI-PEG 20 preparations were evaluated under two different storage temperatures. The stability parameters that were evaluated include appearance before and after reconstitution, enzyme activity, and PEG number. Table E11 below provides a summary of the PEG number and formulation tested in this study.

TABLE E11

| Form | PEG | Composition/ | | | | | |
|---|---|---|---|---|---|---|---|
| ID | number | Buffer | Bulking Agent | Stabilizer | Plasticizer | Surfactant | Chelator |
| P204 | High (5) | 150 mM sodium | 4.5% Sucrose | 0.5% | 0.25% | 0.01% | 0.05% |
| | | phosphate | 4.5% | Glycine | Glycerol | Tween 80 | EDTA |
| | | pH 6.8 | Trehalose | | | | |
| C204 | Low (2) | 150 mM sodium | 4.5% Sucrose | 0.5% | 0.25% | 0.01% | 0.05% |
| | | phosphate | 4.5% | Glycine | Glycerol | Tween 80 | EDTA |
| | | pH 6.8 | Trehalose | | | | |

The lyophilization process was as follows. Samples were frozen to make sure the entire formulation was in its solid phase. Then, each vial of sample was freeze dried using a Virtis Freeze Mobile 25 EL at −78° C. to −85° C. and 50-120 mTorr for approximately 24 hours to allow as much water as possible to sublime.

As mentioned previously, the storage temperature for each of these lyophilized products was either at 2-8° C. or room temperature. The two storage temperatures and storage duration are listed in Table E12 below.

TABLE E12
Storage Conditions.

| | Duration | | |
|---|---|---|---|
| Temperature | 0 months | 1 months | 3 months |
| 2-8° C. | N/A | N/A | X |
| Room Temperature | X | X | X |

Upon lyophilization and storage at the respective temperatures, the lyophiles were analyzed. After lyophilization, the appearance of the product was a white and fluffy cake. After reconstitution with water, both Formulation ID P204 and C204 were clear and particle free. The reconstitution time for each of P204 and C204 was less than 1 minute.

Enzyme activity, reverse phase (RP) chromatography, and SDS-PAGE analyses were performed on the lyophilized and reconstituted products. As shown below in Table E13, storage of lyophiles at 2-8° C. resulted in greater retention of ADI enzyme activity relative to storage at room temperature storage. For example, P204 retained about 92% of its ADI enzyme activity after storage at 2-8° C. for 3 months, and about 86% of its activity after storage at room temperature for 3 months (relative to 0 month timepoint). C204 retained about 96% of its ADI enzyme activity after storage at 2-8° C. for 3 months, and about 74% of its enzyme activity after storage at room temperature for 3 months (relative to 0 month timepoint).

TABLE E13
Lyophilized/Reconstituted Product Enzyme Activity (IU/mg)

| Temperature\ | P204 | | | C204 | | |
|---|---|---|---|---|---|---|
| Time | 0 mon | 1 mon | 3 mons | 0 mon | 1 mon | 3 mons |
| 2-8° C. | N/A | N/A | 22.4 | N/A | N/A | 68.3 |
| | | | (92%) | | | (96%) |
| Room | 24.3 | 21.2 | 20.8 | 70.9 | 61.3 | 52.2 |
| Temperature | (100%) | (87%) | (86%) | (100%) | (86%) | (74%) |

RP chromatography was used to analyze PEG number. The number of PEG(s) per protomer before and after lyophilization and reconstitution did not change significantly as shown below in Table E14. The lyophilization process and storage did not result in any decoupling of PEG. SDS-PAGE analysis also showed no significant change in the number of PEG(s) per ADI protomer.

TABLE E14
Lyophilized/Reconstituted PEG(s) Number per Protomer

| Temperature\ | P204 | | | C204 | | |
|---|---|---|---|---|---|---|
| Time | 0 mon | 1 mon | 3 mons | 0 mon | 1 mon | 3 mons |
| 2-8° C. | N/A | N/A | 4.64 | N/A | N/A | 1.32 |
| | | | (100.2%) | | | (86%) |
| Room | 4.63 | 4.62 | 4.57 | 1.53 | 1.49 | 1.53 |
| Temperature | (100%) | (99.8%) | (98.7%) | (100%) | (97.4%) | (100%) |

Each of P204 and C204 were clear and free of particles after reconstitution with solvent, and reconstitution occurred in less than 1 minute. Moreover, for each of P204 and C204, the loss of ADI enzyme activity that occurred after storage at room temperature was greater than that observed after storage at 2-8° C. Furthermore, for both storage temperatures, the number of PEGs per ADI protomer remained unaltered.

### Example 5

Studies were performed to evaluate the storage conditions of ADI-PEG 20 formulation consisting of ADI covalently bonded with PEG by a water-labile linker. The storage conditions were 2-8° C. for up to 28 months. The stability parameters that were evaluated include appearance, PEG number, free PEG, and ADI enzyme activity. Table E12 below provides a summary of the formulation tested in this study.

TABLE E12

| Formulation ID | Composition/Buffer | pH |
|---|---|---|
| HS | 130 mM sodium chloride, | 6.8 |
| | 35 mM Histidine buffer | |

Upon lyophilization and storage at the evaluated condition, the Formulation ID HS was analyzed. After lyophilization and storage at 2-8° C. for 28 months, the appearance of the formulation was a uniform well-packed white cake with no gross melt-back. After reconstitution with water for lyophilized product stored for 28 months, the Formulation ID HS was clear and particle free. The reconstitution time was about 40 seconds.

Evaluation of enzyme activity was conducted on the lyophilized product stored at 2-8° C. and compared with the activity for product that was stored frozen at −70° C. Enzyme activity was fully retained when storing the lyophile at 2-8° C. for 28 months. Results are shown below in Table E13.

TABLE E13
Lyophilized Product Enzyme Activity (IU/mg)

| | Control frozen | Formulation ID HS | |
|---|---|---|---|
| | product stored | stored at 2-8° C. | |
| | at −70° C. | For 28 months | |
| Storage condition | 6.0 | 6.3 | 105% remaining |

RP chromatography and SEC chromatography were used to analyze PEG number and free PEG concentration (mg/mL), respectively. The number of PEG(s) per protomer and free PEG before and after lyophilization and after 28 months of storage at 2-8° C. did not change significantly as is shown below in Table E14. It is obvious that the lyophilization process and up to 28 months of storage did not result in decoupling of PEG from the enzyme.

TABLE E14
Lyophilized product PEG(s) Number per Monomer and free PEG

| Sample tested | PEG/Monomer | Free PEG (mg/mL) |
|---|---|---|
| Control | 5.33 | 0.2 |
| Formulation ID HS | 5.51 | 0.1 |

In summary, formulation HS was clear and free of particles after reconstitution with solvent and reconstitution occurred in less than 1 minute. In addition, this study also shows that the decoupling of covalently attached PEG does not occur during storage and the enzyme is fully active upon reconstitution. ADI covalently linked with a water labile PEG before and after lyophilization, and reconstitution in the storage condition of 2-8° C. was stable for at least 28 months.

1. A lyophilized formulation, comprising arginine deiminase-polyethylene glycol 20K (ADI-PEG 20), wherein the lyophilized formulation is at a pharmaceutically-acceptable pH, a histidine buffer at a concentration of about 1 to about 50 mM, sodium chloride, and sucrose at about 1 wt % to about 20 wt %, and wherein the ADI-PEG 20 retains at least 85%, 90% or 95% arginine deiminase (ADI) activity relative to a corresponding ADI-PEG 20 in a non-lyophilized liquid control composition after being stored as a lyophilized formulation over 3 months at a temperature of 2-8° C.

2. The lyophilized formulation of claim 1, wherein the lyophilized formulation comprising the ADI-PEG 20 is stored as a lyophilized formulation for over 3 months and less than 28 months.

3. The lyophilized formulation of claim 2, wherein the lyophilized formulation comprising the ADI-PEG 20 is stored as a lyophilized formulation for 28 months.

4. The lyophilized formulation of claim 1, further comprising a bulking agent or a high glass transition temperature excipient.

5. The lyophilized formulation of claim 4, wherein the high glass transition temperature excipient is trehalose.

6. The lyophilized formulation of claim 1, wherein the sucrose at about 1% to about 10%.

7. The lyophilized formulation of claim 1, wherein the pH is about 6.0 to about 7.0±0.1.

8. The lyophilized formulation of claim 6, wherein the sodium chloride is less than 150 mM while the sucrose is about 1 to about 5 wt %.

9. The lyophilized formulation of claim 1, wherein the ADI-PEG 20 comprises an amino acid sequence that is at least 80, 95, 90, 95, 96, 97, 98, 99, or 100% identical to a sequence selected from SEO ID NOs: 1-56.

10. The lyophilized formulation of claim 1, wherein the ADI-PEG 20 comprises one or more water-labile linkers which covalently attach the ADI and PEG.

11. A method of reconstituting the lyophilized formulation of claim 1, comprising adding a pharmaceutically acceptable solvent to the lyophilized formulation to form a reconstituted liquid composition.

12. The method of claim 11, wherein the structure of ADI-PEG 20 of the reconstituted liquid composition returns to its ADI-PEG 20 original state.

13. The method of claim 11, wherein the lyophilized formulation is reconstituted to a substantially aggregate-free solution of about 5-20 mg/ml ADI-PEG 20 in a time of less than about one or two minutes.

14. The method of claim 11, wherein the specific ADI enzyme activity of the ADI-PEG 20 is about 5.0 to about 120 IU/mg, or about 5.5, 6, 6.5, 7, 7.5, 8, 8.5, 9.0, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 35, 40, 45, 50, 55, 60, 65, 70, 75, 80, 85, 90, 95, 100, 105, 110, 115, or 120 IU/mg.

15. The method of claim 11, wherein the ADI-PEG has an osmolality of about 50 mOsm/kg to about 500 mOsm/kg, or about 50, 55, 60, 65, 70, 75, 80, 85, 90, 95, 100, 105, 110, 120, 125, 130, 135, 140, 145, 150, 155, 160, 165, 170, 175, 180, 185, 190, 195, 200, 205, 210, 220, 225, 230, 235, 240, 245, 250, 255, 260, 265, 270, 275, 280, 285, 290, 295, 300, 305, 310, 320, 325, 330, 335, 340, 345, 350, 355, 360, 365, 370, 375, 380, 385, 390, 395, 400, 405, 410, 420, 425, 430, 435, 440, 445, 450, 455, 460, 465, 470, 475, 480, 485, 490, 495, or about 500 mOsm/kg.

16. The method of claim 11, wherein the solvent is water.

17. A patient care kit comprising the lyophilized formulation of claim 1, and optionally a pharmaceutically acceptable solvent.

18. The patient care kit of claim 17, wherein the solvent is water.

19. The patient care kit of claim 17, wherein the ADI-PEG has an osmolality of about 50 mOsm/kg to about 500 mOsm/kg, or about 50, 55, 60, 65, 70, 75, 80, 85, 90, 95, 100, 105, 110, 120, 125, 130, 135, 140, 145, 150, 155, 160, 165, 170, 175, 180, 185, 190, 195, 200, 205, 210, 220, 225, 230, 235, 240, 245, 250, 255, 260, 265, 270, 275, 280, 285, 290, 295, 300, 305, 310, 320, 325, 330, 335, 340, 345, 350, 355, 360, 365, 370, 375, 380, 385, 390, 395, 400, 405, 410, 420, 425, 430, 435, 440, 445, 450, 455, 460, 465, 470, 475, 480, 485, 490, 495, or about 500 mOsm/kg.

20. The patient care kit of claim 19, wherein the ADI-PEG has an osmolality of about 300 mOsm/kg.
